(19) United States
(12) Patent Application Publication
(10) Pub. No.: US 2010/0260792 A1
(43) Pub. Date: Oct. 14, 2010

(54) RESPIRATORY SYNCYTIAL VIRUS VACCINE BASED ON CHIMERIC PAPILLOMAVIRUS VIRUS-LIKE PARTICLES OR CAPSOMERES

(75) Inventors: Yoshihiko Murata, Pittsford, NY (US); Robert C. Rose, Geneseo, NY (US); Edward E. Walsh, Pittsford, NY (US); Ann R. Falsey, Rochester, NY (US)

Correspondence Address: NIXON PEABODY LLP - PATENT GROUP, 1100 CLINTON SQUARE, ROCHESTER, NY, 14604, US

(73) Assignee: UNIVERSITY OF ROCHESTER, Rochester, NY (US)

(21) Appl. No.: 12/738,991

(22) PCT Filed: Oct. 22, 2008
(86) PCT No.: PCT/US08/80822

§ 371 Date: Jun. 28, 2010

Related U.S. Application Data
(60) Provisional application No. 60/981,719, filed on Oct. 22, 2007.

(51) Int. Cl. A61K 39/155; C07K 14/025; C12N 15/63; C12N 1/00; C12P 21/02; A61K 39/12; C07K 1/00; C07K 14/135; A61P 37/04; A61P 31/20; A61P 31/14

(52) U.S. Cl. 424/192.1; 530/350; 435/320.1; 435/243; 435/69.7; 424/196.11; 530/402

(57) ABSTRACT

The present invention is directed to a chimeric papillomavirus virus-like particle (VLP) or capsomere including an L1 polypeptide and, optionally, an L2 polypeptide, and a respiratory syncytial virus (RSV) protein or polypeptide fragment thereof comprising a first epitope, where the RSV protein or polypeptide fragment thereof is attached to one or both of the L1 and L2 polypeptides. Chimeric proteins, genetic constructions, and recombinant vectors and host cells suitable for expression of the constructs and making of the chimeric VLPs or capsomeres are also disclosed. Use of the VLPs or capsomeres, or a pharmaceutical composition containing the same, is contemplated for inducing a protective immune response against RSV.

This application claims the benefit of U.S. Provisional Patent Application Ser. No. 60/981,719, filed Oct. 22, 2007, which is hereby incorporated by reference in its entirety.

### FIELD OF THE INVENTION

The present invention is directed to Respiratory Syncytial Virus (“RSV”) vaccine based on chimeric papillomavirus virus-like particles or capsomeres.

### BACKGROUND OF THE INVENTION

RSV remains a predictable cause of respiratory tract illness in persons of all ages and is the most important cause of lower respiratory tract infections in infants and children (Hall C B et al., “Respiratory Syncytial Virus. In: Mandell G L, Bennett J E, Dolin R, eds. Principles and Practices of Infectious Disease. Vol 6th. Philadelphia: Elsevier Churchill Livingstone; 2008-2026 (2004)). Following primary RSV infection, which generally occurs by age 2, immunity to RSV remains incomplete and frequent re-infections occur throughout life, with the most severe infections occurring at the extremes of age (Hall et al., “Immunity to and Frequency of Reinfection with Respiratory Syncytial Virus,” J Infect Dis 163:693-698 (1991)).

In the United States, RSV is estimated to cause ˜126,000 annual hospitalizations and ˜300 deaths among infants ≦1 yr of age (Hall C B., “Respiratory Syncytial Virus and Parainfluenza Virus,” N Engl J Med 334:1917-1928 (2001) and Thompson et al., “Mortality Associated with Influenza and Respiratory Syncytial Virus in the United States,” J Am Med Assoc 289:179-186 (2003)). For the pediatric population, ribavirin is the only approved therapeutic agent and of debatable benefit, while the only available prophylactic agent is the humanized monoclonal antibody (mAb) palivizumab, which is currently licensed only for use in high-risk infants (The IMpact-RSV Study Group, “Palivizumab, a Humanized Respiratory Syncytial Virus Monoclonal Antibody, Reduces Hospitalization from Respiratory Syncytial Virus Infection in High-risk Infants,” Pediatrics 102:531-537 (1998); Hall et al., “Aerosolized Ribavirin Treatment of Infants with Respiratory Syncytial Viral Infection,” N Engl J Med 308:1443-1447 (1983); Hall et al., “Ribavirin Treatment of Respiratory Syncytial Viral Infection in Infants with Underlying Cardiopulmonary Disease,” JAMA 254:3047-3051 (1985); and Rodriguez et al., “Prospective Follow-up and Pulmonary Functions from a Placebo-controlled Randomized Trial of Ribavirin Therapy in Respiratory Syncytial Virus Bronchiolitis,” Arch Pediatr Adolesc Med 153:469-474 (1999)).

RSV also accounts for >80,000 hospitalizations and >13,000 deaths each winter among adults who are elderly or have underlying cardiopulmonary and/or immuno-suppressive conditions (Thompson et al., “Mortality Associated with Influenza and Respiratory Syncytial Virus in the United States,” J Am Med Assoc 289:179-186 (2003); Dowell et al., “Respiratory Syncytial Virus is an Important Cause of Community-acquired Lower Respiratory Infection among Hospitalized Adults,” J Infect Dis 174:456-462 (1996); Falsey et al., “Respiratory Syncytial Virus Infection in Elderly Adults,” Drugs Aging 22:577-587 (2005)). Because of the substantial disease burden and limited therapeutic and prophylactic options, development of an RSV vaccine continues to be a high priority.

Various strategies have been pursued to develop an effective and safe RSV vaccine including: 1) inactivated virus preparations; 2) live attenuated/genetically engineered viruses; and 3) purified subunit vaccines. Each approach is summarized below.

One RSV vaccine approach involved inactivated virus preparations. The first RSV vaccine trial, performed nearly 40 years ago, employed a parenterally administered, formalin-inactivated whole virus preparation (Fulginiti et al., “Respiratory Virus Immunization. I. A Field Trial of Two Inactivated Respiratory Virus Vaccines; an Aqueous Trivalent Parainfluenza Virus Vaccine and an Alum-precipitated Respiratory Syncytial Virus Vaccine,” Am J Epidemiol 89:435-448 (1969)). This strategy failed to induce neutralizing antibodies or protect vaccinated infants and led paradoxically to enhanced disease severity when RSV infection occurred during the subsequent winter season (Murphy et al., “Formalin-inactivated Respiratory Syncytial Virus Vaccine Induces Antibodies to the Fusion Glycoprotein that are Deficient in Fusion-inhibiting Activity,” J Clin Microbiol 26:1595-1597 (1988); Chin et al., “Field Evaluation of a Respiratory Syncytial Virus Vaccine and a Trivalent Parainfluenza Virus Vaccine in a Pediatric Population,” Am J Epidemiol 89:449-463 (1969); Kapikian et al., “An Epidemiologic Study of Altered Clinical Reactivity to Respiratory Syncytial (RS) Virus Infection in Children Previously Vaccinated with an Inactivated R S Virus Vaccine,” Am J Epidemiol 89:405-421 (1969); Kim et al., “Respiratory Syncytial Virus Disease in Infants Despite Prior Administration of Antigenic Inactivated Vaccine,” Am J Epidemiol 89:422-434 (1969)). This outcome was attributed to the denaturation of neutralizing epitopes on the F protein as well as vaccine-induced priming of Th2 CD4+ T-cells (Murphy et al., “Formalin-inactivated Respiratory Syncytial Virus Vaccine Induces Antibodies to the Fusion Glycoprotein that are Deficient in Fusion-inhibiting Activity,” J Clin Microbiol 26:1595-1597 (1988); Prince et al., “Enhancement of Respiratory Syncytial Virus Pulmonary Pathology in Cotton Rats by Prior Intramuscular Inoculation of Formalin-inactivated Virus,” J Virol 57:721-728 (1986); Peebles et al., “Respiratory Syncytial Virus (RSV)-induced Airway Hyperresponsiveness in Allergically Sensitized Mice is Inhibited by Live RSV and Exacerbated by Formalin-inactivated RSV,” J Infect Dis 182:671-677 (2000)). An ensuing trial using parenterally administered live virus also failed to provide protection, although enhanced RSV disease did not occur (Buynak et al., “Live Respiratory Syncytial Virus Vaccine Administered Parenterally,” Proc Soc Exp Biol Med 157:636-642 (1978); Buynak et al., “Further Investigations of Live Respiratory Syncytial Virus Vaccine Administered Parenterally,” Proc Soc Exp Biol Med 160:272-277 (1979); Belshe et al., “Parenteral Administration of Live Respiratory Syncytial Virus Vaccine: Results of a Field Trial,” Infect Dis 145:311-319 (1982)).

Another RSV vaccine approach involved live attenuated/genetically engineered viruses. In animal models, including BALB/c mice, intranasal administration of live attenuated RSV strains can induce mucosal and humoral antibody responses and Th1 dominant local and systemic cytotoxic T-cell lysis (“CTL”) responses (Openshaw et al., “Immune Responses and Disease Enhancement During Respiratory Syncytial Virus Infection,” Clin Microbiol Rev 18:541-555 (2005); Crowe et al., “A Further Attenuated Derivative of a Cold-passaged Temperature-sensitive Mutant of Human Respiratory Syncytial Virus Retains Immunogenicity and Protective Efficacy Against Wild-type Challenge in Seronegative Chimpanzees,” Vaccine 12:783-790 (1994); Crowe et al., “Live Subgroup B Respiratory Syncytial Virus Vaccines that are Attenuated, Genetically Stable, and Immunogenic in Rodents and Nonhuman Primates,” J Infect Dis 173:829-839 (1996); Crowe et al., “Satisfactorily Attenuated and Protective Mutants Derived from a Partially Attenuated Cold-Passaged Respiratory Syncytial Virus Mutant by Introduction of Additional Attenuating Mutations During Chemical Mutagenesis,” Vaccine 12:691-699 (1994); Crowe et al., “Cold-passaged, Temperature-sensitive Mutants of Human Respiratory Syncytial Virus (RSV) are Highly Attenuated, Immunogenic, and Protective in Seronegative Chimpanzees, even when RSV Antibodies are Infused Shortly before Immunization,” Vaccine 13:847-855 (1995); Crowe et al., “The Live Attenuated Subgroup B Respiratory Syncytial Virus Vaccine Candidate RSV 2B33F is Attenuated and Immunogenic in Chimpanzees, but Exhibits Partial Loss of the ts Phenotype Following Replication In Vivo,” Virus Res 59:13-22 (1999); Peebles et al., “Pathogenesis of Respiratory Syncytial Virus Infection in the Murine Model,” Proc Am Thorac Soc 2:110-115 (2005)). Immunization of mice with live RSV or with replicating vectors encoding RSV F protein induces a Th1 dominant response with neutralizing antibody and CD8+ CTL responses that are associated with minimal pulmonary pathology upon virus challenge (Pemberton et al., “Cytotoxic T Cell Specificity for Respiratory Syncytial Virus Proteins: Fusion Protein is an Important Target Antigen,” J Gen Virol 68 (Pt 8):2177-2182 (1987); Openshaw et al., “Immune Responses and Disease Enhancement During Respiratory Syncytial Virus Infection,” Clin Microbiol Rev 18:541-555 (2005); Buynak et al., “Live Respiratory Syncytial Virus Vaccine Administered Parenterally,” Proc Soc Exp Biol Med 157:636-642 (1978); Buynak et al., “Further Investigations of Live Respiratory Syncytial Virus Vaccine Administered Parenterally,” Proc Soc Exp Biol Med 160:272-277 (1979); Peebles et al., “Pathogenesis of Respiratory Syncytial Virus Infection in the Murine Model,” Proc Am Thorac Soc 2:110-115 (2005)). Limited clinical trials have been performed with live virus vaccines selected for temperature-sensitive (ts) or cold-passaged (cp) attenuated phenotypes (Chanock et al., “Use of Temperature-sensitive and Cold-adapted Mutant Viruses in Immunoprophylaxis of Acute Respiratory Tract Disease,” Rev Infect Dis 2:421-432 (1980); Connors et al., “A Cold-passaged, Attenuated Strain of Human Respiratory Syncytial Virus Contains Mutations in the F and L Genes,” Virology 208:478-484 (1995); Kim et al., “Safety and Antigenicity of Temperature Sensitive (TS) Mutant Respiratory Syncytial Virus (RSV) in Infants and Children,” Pediatrics 52:56-63 (1973)). However, the modified virus strains suffered from key limitations such as insufficiently attenuated or unstable phenotype and lack of immunogenicity. For these reasons, they were not pursued (Wright et al., “Evaluation of a Live, Cold-passaged, Temperature-sensitive, Respiratory Syncytial Virus Vaccine Candidate in Infancy,” J Infect Dis 182:1331-1342 (2000); Gonzalez et al., “Evaluation of the Live Attenuated cpts 248/404 RSV Vaccine in Combination with a Subunit RSV Vaccine (PFP-2) in Healthy Young and Older Adults,” Vaccine 18:1763-1772 (2000)).

Recent advances using reverse genetics have led to additional live virus derivatives, including: 1) combinatorial arrangement of known attenuating mutations to generate stable is or cp phenotypes; 2) deletion of specific viral genes to confer phenotypic attenuation and prevent spontaneous reversion (i.e., deletion of NS1 and/or NS2 genes which block type I interferon response); 3) genetic rearrangements to optimize the expression of protective antigens such as the F protein; and 4) creation of bovine parainfluenza virus—human RSV chimeric viruses (Whitehead et al., “Recombinant Respiratory Syncytial Virus (RSV) Bearing a Set of Mutations from Cold-passaged RSV is Attenuated in Chimpanzees,” J Virol 72:4467-4471 (1998); Whitehead et al., “Replacement of the F and G Proteins of Respiratory Syncytial Virus (RSV) Subgroup A with Those of Subgroup B Generates Chimeric Live Attenuated RSV Subgroup B Vaccine Candidates,” J Virol 73:9773-9780 (1999); Whitehead et al., “Addition of a Missense Mutation Present in the L Gene of Respiratory Syncytial Virus (RSV) cpts530/1030 to RSV Vaccine Candidate cpts248/404 Increases its Attenuation and Temperature Sensitivity,” J Virol 73:871-877 (1999); Whitehead et al., “A Single Nucleotide Substitution in the Transcription Start Signal of the M2 Gene of Respiratory Syncytial Virus Vaccine Candidate cpts248/404 is the Major Determinant of the Temperature-sensitive and Attenuation Phenotypes,” Virology 247:232-239 (1998); Whitehead et al., “Recombinant Respiratory Syncytial Virus Bearing a Deletion of Either the NS2 or SH Gene is Attenuated in Chimpanzees,” J Virol 73:3438-3442 (1999); Teng et al., “Altered Growth Characteristics of Recombinant Respiratory Syncytial Viruses Which do Not Produce NS2 Protein,” J Virol 73:466-473 (1999); Schmidt et al., “Mucosal Immunization of Rhesus Monkeys Against Respiratory Syncytial Virus Subgroups A and B and Human Parainfluenza Virus Type 3 by Using a Live cDNA-derived Vaccine Based on a Host Range-attenuated Bovine Parainfluenza Virus type 3 Vector Backbone,” J Virol 76:1089-1099 (2002); Skiadopoulos et al., “Attenuation of the Recombinant Human Parainfluenza Virus Type 3 cp45 Candidate Vaccine Virus is Augmented by Importation of the Respiratory Syncytial Virus cpts530 L Polymerase Mutation,” Virology 260:125-135 (1999); Schmidt et al., “Recombinant Bovine/human Parainfluenza Virus Type 3 (B/HPIV3) Expressing the Respiratory Syncytial Virus (RSV) G and F Proteins can be Used to Achieve Simultaneous Mucosal Immunization Against RSV and HPIV3,” J Virol 75:4594-4603 (2001); Crowe et al., “Acquisition of the is Phenotype by a Chemically Mutagenized Cold-passaged Human Respiratory Syncytial Virus Vaccine Candidate Results from the Acquisition of a Single Mutation in the Polymerase (L) Gene,” Virus Genes 13:269-273 (1996); Karron et al., “Respiratory Syncytial Virus (RSV) SH and G Proteins are Not Essential for Viral Replication In Vitro: Clinical Evaluation and Molecular Characterization of a Cold-passaged, Attenuated RSV Subgroup B Mutant,” Proc Natl Acad Sci USA 94:13961-13966 (1997); Karron et al., “Evaluation of Two Live, Cold-passaged, Temperature-sensitive Respiratory Syncytial Virus Vaccines in Chimpanzees and in Human Adults, Infants, and Children,” J Infect Dis 176:1428-1436 (1997); Karron et al., “Identification of a Recombinant Live Attenuated Respiratory Syncytial Virus Vaccine Candidate That is Highly Attenuated in Infants,” J Infect Dis 191:1093-1104 (2005)). These candidate vaccines have all been assessed in animals or in clinical trials, but their development has been hampered by poor immunogenicity or unacceptable levels of adverse reactogenicity. Moreover, live replicating vectors face potential safety issues that are inherently associated with such vaccine platforms (Bembridge et al., “Respiratory Syncytial Virus Infection of Gene Gun Vaccinated Mice Induces Th2-driven Pulmonary Eosinophilia Even in the Absence of Sensitisation to the Fusion (F) or Attachment (G) Protein,” Vaccine 19:1038-1046 (2000); Wyatt et al., “Priming and Boosting Immunity to Respiratory Syncytial Virus by Recombinant Replication-defective Vaccinia Virus MVA,” Vaccine 18:392-397 (1999)).

Another RSV vaccine approach involved subunit vaccines. RSV-derived proteins, including F and F/G chimeric proteins produced in prokaryotic and eukaryotic systems or via live replicating vectors (e.g., adenovirus, vaccinia virus) have been tested in animal models (Olmsted et al., “Evaluation in Non-human Primates of the Safety, Immunogenicity and Efficacy of Recombinant Vaccinia Viruses Expressing the F or G Glycoprotein of Respiratory Syncytial Virus,” Vaccine 6:519-524 (1988); Murphy et al., “Immunization of Cotton Rats with the Fusion (F) and Large (G) Glycoproteins of Respiratory Syncytial Virus (RSV) Protects Against RSV Challenge without Potentiating RSV Disease,” Vaccine 7:533-540 (1989); Trudel et al., “Synthetic Peptides Corresponding to the F Protein of RSV Stimulate Murine B and T Cells but Fail to Confer Protection,” Arch Virol 117:59-71 (1991); Wathen et al., “Vaccination of Cotton Rats with a Chimeric FG Glycoprotein of Human Respiratory Syncytial Virus Induces Minimal Pulmonary Pathology on Challenge,” J Infect Dis 163:477-482 (1991); Piedra et al., “Safety and Immunogenicity of the PFP Vaccine Against Respiratory Syncytial Virus (RSV): The Western Blot Assay Aids in Distinguishing Immune Responses of the PFP Vaccine from RSV Infection,” Vaccine 13:1095-1101 (1995); Falsey et al., “Safety and Immunogenicity of a Respiratory Syncytial Virus Subunit Vaccine (PFP-2) in Ambulatory Adults over Age 60,” Vaccine 14:1214-1218 (1996)). However, immunization of mice with a subunit F protein vaccine induces a Th2 dominant response without associated CD8+ CTL and paradoxically leads to increased pathological changes in the lungs even if neutralizing antibodies are present (Bembridge et al., “Priming with a Secreted Form of the Fusion Protein of Respiratory Syncytial Virus (RSV) Promotes Interleukin-4 (IL-4) and IL-5 Production but Not Pulmonary Eosinophilia Following RSV Challenge,” J Virol 73:10086-10094 (1999); Bembridge et al., “Recombinant Vaccinia Virus Coexpressing the F Protein of Respiratory Syncytial Virus (RSV) and Interleukin-4 (IL-4) Does Not Inhibit the Development of RSV-specific Memory Cytotoxic T Lymphocytes, Whereas Priming is Diminished in the Presence of High Levels of IL-2 or Gamma Interferon, J Virol 72:4080-4087 (1998)). Thus, this approach to vaccinate RSV-naïve infants has not been extensively pursued because of various theoretical risks (e.g., Th2-dominant response) of potentiating subsequent RSV re-infections (Connors et al., “Cotton Rats Previously Immunized with a Chimeric RSV FG Glycoprotein Develop Enhanced Pulmonary Pathology when Infected with RSV, a Phenomenon not Encountered Following Immunization with Vaccinia—RSV Recombinants or RSV,” Vaccine 10:475-484 (1992); Belshe et al., “Immunogenicity of Purified F Glycoprotein of Respiratory Syncytial Virus: Clinical and Immune Responses to Subsequent Natural Infection in Children,” J Infect Dis 168:1024-1029 (1993); Corvaia et al., “Challenge of BALB/c Mice with Respiratory Syncytial Virus Does Not Enhance the Th2 Pathway Induced After Immunization with a Recombinant G Fusion Protein, BBG2NA, in Aluminum Hydroxide,” J Infect Dis 176:560-569 (1997).

As summarized above, various efforts towards RSV vaccine development have encountered technical limitations and/or unanticipated immune responses which have precluded successful clinical development. Based on preclinical models of RSV infection (mostly in rodents) and limited clinical data of previous vaccine candidates, an optimal RSV vaccine for use in pediatric and possibly in adult populations should fulfill the following criteria: 1) generate a Th1-dominant immune response and thereby avoid the pulmonary pathology associated with Th2 response; 2) generate neutralizing antibodies against RSV-encoded proteins; 3) generate Th1-associated CTL response; and 4) circumvent the potential adverse events associated with live vaccine platforms.

The present invention is directed to overcoming these and other deficiencies in the art.

### SUMMARY OF THE INVENTION

A first aspect of the present invention relates to a chimeric papillomavirus virus-like particle (VLP) or capsomere including an L1 polypeptide and, optionally, an L2 polypeptide, and a respiratory syncytial virus (RSV) protein or polypeptide fragment thereof comprising a first epitope, where the RSV protein or polypeptide fragment thereof is attached to one or both of the L1 and L2 polypeptides.

A second aspect of the present invention relates to a pharmaceutical composition including a chimeric papillomavirus VLP or capsomere of the present invention and a pharmaceutically acceptable carrier.

A third aspect of the present invention relates to a method of inducing an immune response against respiratory syncytial virus (RSV) including administering a chimeric VLP or capsomere of the present invention or pharmaceutical composition of the present invention to an individual in an amount effective to induce an immune response against RSV.

A fourth aspect of the present invention relates to a method of preventing RSV infection that includes administering a chimeric VLP or capsomere of the present invention (or pharmaceutical composition of the present invention) to an individual in an amount effective to prevent RSV infection.

A fifth aspect of the present invention relates to a genetic construct encoding one or both of an L1 polypeptide-RSV polypeptide chimeric protein and an L2 polypeptide-RSV polypeptide chimeric protein.

A sixth aspect of the present invention relates to a recombinant vector including a genetic construct according to the present invention. This aspect of the invention also relates to a recombinant organism that includes a host cell or a recombinant vector of the present invention.

A seventh aspect of the present invention relates to a chimeric protein including a papillomavirus L1 or L2 polypeptide and an RSV polypeptide linked via an in-frame gene fusion.

An eighth aspect of the present invention relates to a method of making a chimeric VLP or capsomere of the present invention. This method includes the step of introducing a genetic construct or recombinant vector of the present invention into a host cell under conditions effective to express either (i) a fusion protein comprising an L1 polypeptide and RSV polypeptide, and optionally an L2 polypeptide; or (ii) an L1 polypeptide and a fusion protein comprising an L2 polypeptide and an RSV polypeptide, whereby the expressed polypeptide(s) self-assemble into the chimeric VLP or capsomere.

A ninth aspect of the present invention relates to a method of making a chimeric VLP or capsomere of the present invention. This method includes the steps of first exposing a papillomavirus VLP or capsomere to a bi-functional linker molecule under conditions effective to allow covalent bond formation between the linker molecule and the VLP or capsomere, and then second exposing an RSV polypeptide to the product of said first exposing to allow covalent bond formation between the RSV polypeptide and the bound linker molecule, thereby forming the chimeric VLP or capsomere.

To fulfill the unmet medical need for an RSV vaccine, the present invention harnesses the unique immunological and biophysical properties of papillomavirus capsid proteins as a vaccine platform. A number of different HPV/RSV VLPs and capsomeres have been generated, each bearing a portion of the RSV F protein or G protein fused to either a truncated L2N protein or at the site of a helix 4 deletion of the L1 protein. These chimeric papillomavirus VLPs and capsomeres appear to fulfill the structural and immunological criteria that are required for animal immunogenicity studies. The accompanying examples demonstrate the preparation of these chimeric papillomavirus VLPs and capsomeres and their immunogenicity. The immune sera generated following inoculation was shown to recognize purified RSV F and G proteins in ELISA assays (under non-denaturing conditions). It is therefore expected that these sera will be effective in RSV neutralization assays, and the chimeric VLPs and capsomeres will form an effective vaccine.

### BRIEF DESCRIPTION OF THE DRAWINGS

FIG. 1 is a schematic diagram of the RSV F Protein. HRA and HRB represent the heptad-repeat domains A and B; cross-hatched areas represent the predicted hydrophobic domains based on amino acid sequence; TM represents the transmembrane domain; and FP represents the fusion peptide). The primary Fo protease cleavage sites are indicated by vertical arrows; the right arrow represents two cleavage sites at aa 109 and 136. Portions of the F protein used herein are depicted as rectangles numbered 1, 2, 3, and 4 with amino acid numbers shown as flanking the respective boxes. Within each F fragment, epitopes that elicit CTL responses or are recognized by neutralizing antibodies (Neut) are indicated by arrows or lines, respectively.

FIGS. 2A-B show the nucleotide (SEQ ID NO: 1) and amino acid (SEQ ID NO: 2) sequences, respectively, of the F protein from RSV, strain RGH.

FIGS. 3A-B show the nucleotide (SEQ ID NO: 3) and amino acid (SEQ ID NO: 4) sequences, respectively, of the G protein from RSV, strain RGH.

FIGS. 4A-B show the nucleotide (SEQ ID NO: 5) and amino acid (SEQ ID NO: 6) sequences, respectively, for a fusion protein including a full-length HPV-16 L1 polypeptide and an RSV F polypeptide consisting of residues 23-122 (shown in bold).

FIGS. 5A-B show the nucleotide (SEQ ID NO: 7) and amino acid (SEQ ID NO: 8) sequences, respectively, for a fusion protein including a full-length HPV-16 L1 polypeptide and an RSV F polypeptide consisting of residues 154-222 (shown in bold).

FIGS. 6A-B show the nucleotide (SEQ ID NO: 9) and amino acid (SEQ ID NO: 10) sequences, respectively, for a fusion protein including a full-length HPV-16 L1 polypeptide and an RSV F polypeptide consisting of residues 226-378 (shown in bold).

FIGS. 7A-B show the nucleotide (SEQ ID NO: 11) and amino acid (SEQ ID NO: 12) sequences, respectively, for a fusion protein including a full-length HPV-16 L1 polypeptide and an RSV F polypeptide consisting of residues 379-523 (shown in bold).

FIGS. 8A-B show the nucleotide (SEQ ID NO: 13) and amino acid (SEQ ID NO: 14) sequences, respectively, for a fusion protein including a full-length HPV-16 L1 polypeptide and an RSV F polypeptide consisting of residues 379-559 (shown in bold).

FIGS. 9A-B show the nucleotide (SEQ ID NO: 15) and amino acid (SEQ ID NO: 16) sequences, respectively, for a fusion protein including a full-length HPV-16 L1 polypeptide and an RSV F polypeptide consisting of residues 254-278 (shown in bold).

FIGS. 10A-B show the nucleotide (SEQ ID NO: 17) and amino acid (SEQ ID NO: 18) sequences, respectively, for a fusion protein including a full-length HPV-16 L1 polypeptide and an RSV F polypeptide consisting of residues 255-278 (shown in bold).

FIGS. 11A-B show the nucleotide (SEQ ID NO: 19) and amino acid (SEQ ID NO: 20) sequences, respectively, for a fusion protein including a full-length HPV-16 L1 polypeptide and an RSV F polypeptide consisting of residues 423-436 (shown in bold).

FIGS. 12A-B show the nucleotide (SEQ ID NO: 21) and amino acid (SEQ ID NO: 22) sequences, respectively, for a fusion protein including a full-length HPV-16 L1 polypeptide and an RSV F polypeptide consisting of residues 249-275 (shown in bold).

FIGS. 13A-B show the nucleotide (SEQ ID NO: 23) and amino acid (SEQ ID NO: 24) sequences, respectively, for a fusion protein including an N-terminal HPV-16 L1 polypeptide and an RSV F polypeptide consisting of residues 23-122 (shown in bold).

FIGS. 14A-B show the nucleotide (SEQ ID NO: 25) and amino acid (SEQ ID NO: 26) sequences, respectively, for a fusion protein including an N-terminal HPV-16 L1 polypeptide and an RSV F polypeptide consisting of residues 154-222 (shown in bold).

FIGS. 15A-B show the nucleotide (SEQ ID NO: 27) and amino acid (SEQ ID NO: 28) sequences, respectively, for a fusion protein including an N-terminal HPV-16 L1 polypeptide and an RSV F polypeptide consisting of residues 226-378 (shown in bold).

FIGS. 16A-B show the nucleotide (SEQ ID NO: 29) and amino acid (SEQ ID NO: 30) sequences, respectively, for a fusion protein including an N-terminal HPV-16 L1 polypeptide and an RSV F polypeptide consisting of residues 379-523 (shown in bold).

FIGS. 17A-B show the nucleotide (SEQ ID NO: 31) and amino acid (SEQ ID NO: 32) sequences, respectively, for a fusion protein including an N-terminal HPV-16 L1 polypeptide and an RSV F polypeptide consisting of residues 379-559 (shown in bold).

FIGS. 18A-B show the nucleotide (SEQ ID NO: 33) and amino acid (SEQ ID NO: 34) sequences, respectively, for a fusion protein including an N-terminal HPV-16 L1 polypeptide and an RSV F polypeptide consisting of residues 254-278 (shown in bold).

FIGS. 19A-B show the nucleotide (SEQ ID NO: 35) and amino acid (SEQ ID NO: 36) sequences, respectively, for a fusion protein including an N-terminal HPV-16 L1 polypeptide and an RSV F polypeptide consisting of residues 255-278 (shown in bold).

FIGS. 20A-B show the nucleotide (SEQ ID NO: 37) and amino acid (SEQ ID NO: 38) sequences, respectively, for a fusion protein including an N-terminal HPV-16 L1 polypeptide and an RSV F polypeptide consisting of residues 423-436 (shown in bold).

FIGS. 21A-B show the nucleotide (SEQ ID NO: 39) and amino acid (SEQ ID NO: 40) sequences, respectively, for a fusion protein including an N-terminal HPV-16 L1 polypeptide and an RSV F polypeptide consisting of residues 249-275 (shown in bold).

FIGS. 22A-B show the nucleotide (SEQ ID NO: 41) and amino acid (SEQ ID NO: 42) sequences, respectively, for a fusion protein including an HPV-16 L1 polypeptide bearing a helix 4 deletion (A404-437) and an RSV F polypeptide consisting of residues 254-278 (shown in bold).

FIGS. 23A-B show the nucleotide (SEQ ID NO: 43) and amino acid (SEQ ID NO: 44) sequences, respectively, for a fusion protein including an HPV-16 L1 polypeptide bearing a helix 4 deletion (A404-437) and an RSV F polypeptide consisting of residues 255-278 (shown in bold).

FIGS. 24A-B show the nucleotide (SEQ ID NO: 45) and amino acid (SEQ ID NO: 46) sequences, respectively, for a fusion protein including an HPV-16 L1 polypeptide bearing a helix 4 deletion (A404-437) and an RSV F polypeptide consisting of residues 423-436 (shown in bold).

FIGS. 25A-B show the nucleotide (SEQ ID NO: 47) and amino acid (SEQ ID NO: 48) sequences, respectively, for a fusion protein including an HPV-16 L1 polypeptide bearing a helix 4 deletion (A404-437) and an RSV F polypeptide consisting of residues 249-275 (shown in bold).

FIGS. 26A-B show the nucleotide (SEQ ID NO: 49) and amino acid (SEQ ID NO: 50) sequences, respectively, for a fusion protein including an HPV-16 L1 polypeptide bearing a helix 4 deletion (A410-429) and an RSV F polypeptide consisting of residues 254-278 (shown in bold).

FIGS. 27A-B show the nucleotide (SEQ ID NO: 51) and amino acid (SEQ ID NO: 52) sequences, respectively, for a fusion protein including an HPV-16 L1 polypeptide bearing a helix 4 deletion (A410-429) and an RSV F polypeptide consisting of residues 255-278 (shown in bold).

FIGS. 28A-B show the nucleotide (SEQ ID NO: 53) and amino acid (SEQ ID NO: 54) sequences, respectively, for a fusion protein including an HPV-16 L1 polypeptide bearing a helix 4 deletion (A410-429) and an RSV F polypeptide consisting of residues 423-436 (shown in bold).

FIGS. 29A-B show the nucleotide (SEQ ID NO: 55) and amino acid (SEQ ID NO: 56) sequences, respectively, for a fusion protein including an HPV-16 L1 polypeptide bearing a helix 4 deletion (A410-429) and an RSV F polypeptide consisting of residues 249-275 (shown in bold).

FIGS. 30A-B show the nucleotide (SEQ ID NO: 57) and amino acid (SEQ ID NO: 58) sequences, respectively, for a fusion protein including a full-length HPV-16 L1 polypeptide and an RSV G polypeptide consisting of residues 154-167 (shown in bold).

FIGS. 31A-B show the nucleotide (SEQ ID NO: 59) and amino acid (SEQ ID NO: 60) sequences, respectively, for a fusion protein including a full-length HPV-16 L1 polypeptide and an RSV G polypeptide consisting of residues 157-168 (shown in bold).

FIGS. 32A-B show the nucleotide (SEQ ID NO: 61) and amino acid (SEQ ID NO: 62) sequences, respectively, for a fusion protein including an N-terminal HPV-16 L1 polypeptide and an RSV G polypeptide consisting of residues 154-167 (shown in bold).

FIGS. 33A-B show the nucleotide (SEQ ID NO: 63) and amino acid (SEQ ID NO: 64) sequences, respectively, for a fusion protein including an N-terminal HPV-16 L1 polypeptide and an RSV G polypeptide consisting of residues 157-168 (shown in bold).

FIGS. 34A-B show the nucleotide (SEQ ID NO: 65) and amino acid (SEQ ID NO: 66) sequences, respectively, for a fusion protein including an HPV-16 L1 polypeptide bearing a helix 4 deletion (A404-437) and an RSV G polypeptide consisting of residues 154-167 (shown in bold).

FIGS. 35A-B show the nucleotide (SEQ ID NO: 67) and amino acid (SEQ ID NO: 68) sequences, respectively, for a fusion protein including an HPV-16 L1 polypeptide bearing a helix 4 deletion (A404-437) and an RSV G polypeptide consisting of residues 157-168 (shown in bold).

FIGS. 36A-B show the nucleotide (SEQ ID NO: 69) and amino acid (SEQ ID NO: 70) sequences, respectively, for a fusion protein including an HPV-16 L1 polypeptide bearing a helix 4 deletion (A410-429) and an RSV G polypeptide consisting of residues 154-167 (shown in bold).

FIGS. 37A-B show the nucleotide (SEQ ID NO: 71) and amino acid (SEQ ID NO: 72) sequences, respectively, for a fusion protein including an HPV-16 L1 polypeptide bearing a helix 4 deletion (A410-429) and an RSV G polypeptide consisting of residues 157-168 (shown in bold).

FIGS. 38A-B show the nucleotide (SEQ ID NO: 73) and amino acid (SEQ ID NO: 74) sequences, respectively, for a fusion protein including a full-length HPV-16 L2 polypeptide and an RSV F polypeptide consisting of residues 23-122 (shown in bold).

FIGS. 39A-B show the nucleotide (SEQ ID NO: 75) and amino acid (SEQ ID NO: 76) sequences, respectively, for a fusion protein including a full-length HPV-16 L2 polypeptide and an RSV F polypeptide consisting of residues 154-222 (shown in bold).

FIGS. 40A-B show the nucleotide (SEQ ID NO: 77) and amino acid (SEQ ID NO: 78) sequences, respectively, for a fusion protein including a full-length HPV-16 L2 polypeptide and an RSV F polypeptide consisting of residues 226-378 (shown in bold).

FIGS. 41A-B show the nucleotide (SEQ ID NO: 79) and amino acid (SEQ ID NO: 80) sequences, respectively, for a fusion protein including a full-length HPV-16 L2 polypeptide and an RSV F polypeptide consisting of residues 379-523 (shown in bold).

FIGS. 42A-B show the nucleotide (SEQ ID NO: 81) and amino acid (SEQ ID NO: 82) sequences, respectively, for a fusion protein including a full-length HPV-16 L2 polypeptide and an RSV F polypeptide consisting of residues 379-559 (shown in bold).

FIGS. 43A-B show the nucleotide (SEQ ID NO: 83) and amino acid (SEQ ID NO: 84) sequences, respectively, for a fusion protein including a full-length HPV-16 L2 polypeptide and an RSV F polypeptide consisting of residues 254-278 (shown in bold).

FIGS. 44A-B show the nucleotide (SEQ ID NO: 85) and amino acid (SEQ ID NO: 86) sequences, respectively, for a fusion protein including a full-length HPV-16 L2 polypeptide and an RSV F polypeptide consisting of residues 255-278 (shown in bold).

FIGS. 45A-B show the nucleotide (SEQ ID NO: 87) and amino acid (SEQ ID NO: 88) sequences, respectively, for a fusion protein including a full-length HPV-16 L2 polypeptide and an RSV F polypeptide consisting of residues 423-436 (shown in bold).

FIGS. 46A-B show the nucleotide (SEQ ID NO: 89) and amino acid (SEQ ID NO: 90) sequences, respectively, for a fusion protein including a full-length HPV-16 L2 polypeptide and an RSV F polypeptide consisting of residues 249-275 (shown in bold).

FIGS. 47A-B show the nucleotide (SEQ ID NO: 91) and amino acid (SEQ ID NO: 92) sequences, respectively, for a fusion protein including an N-terminal HPV-16 L2 polypeptide and an RSV F polypeptide consisting of residues 23-122 (shown in bold).

FIGS. 48A-B show the nucleotide (SEQ ID NO: 93) and amino acid (SEQ ID NO: 94) sequences, respectively, for a fusion protein including an N-terminal HPV-16 L2 polypeptide and an RSV F polypeptide consisting of residues 154-222 (shown in bold).

FIGS. 49A-B show the nucleotide (SEQ ID NO: 95) and amino acid (SEQ ID NO: 96) sequences, respectively, for a fusion protein including an N-terminal HPV-16 L2 polypeptide and an RSV F polypeptide consisting of residues 226-378 (shown in bold).

FIGS. 50A-B show the nucleotide (SEQ ID NO: 97) and amino acid (SEQ ID NO: 98) sequences, respectively, for a fusion protein including an N-terminal HPV-16 L2 polypeptide and an RSV F polypeptide consisting of residues 379-523 (shown in bold).

FIGS. 51A-B show the nucleotide (SEQ ID NO: 99) and amino acid (SEQ ID NO: 100) sequences, respectively, for a fusion protein including an N-terminal HPV-16 L2 polypeptide and an RSV F polypeptide consisting of residues 379-559 (shown in bold).

FIGS. 52A-B show the nucleotide (SEQ ID NO: 101) and amino acid (SEQ ID NO: 102) sequences, respectively, for a fusion protein including an N-terminal HPV-16 L2 polypeptide and an RSV F polypeptide consisting of residues 254-278 (shown in bold).

FIGS. 53A-B show the nucleotide (SEQ ID NO: 103) and amino acid (SEQ ID NO: 104) sequences, respectively, for a fusion protein including an N-terminal HPV-16 L2 polypeptide and an RSV F polypeptide consisting of residues 255-278 (shown in bold).

FIGS. 54A-B show the nucleotide (SEQ ID NO: 105) and amino acid (SEQ ID NO: 106) sequences, respectively, for a fusion protein including an N-terminal HPV-16 L2 polypeptide and an RSV F polypeptide consisting of residues 423-436 (shown in bold).

FIGS. 55A-B show the nucleotide (SEQ ID NO: 107) and amino acid (SEQ ID NO: 108) sequences, respectively, for a fusion protein including an N-terminal HPV-16 L2 polypeptide and an RSV F polypeptide consisting of residues 249-275 (shown in bold).

FIGS. 56A-B show the nucleotide (SEQ ID NO: 109) and amino acid (SEQ ID NO: 110) sequences, respectively, for a fusion protein including a full-length HPV-16 L2 polypeptide and an RSV G polypeptide consisting of residues 154-167 (shown in bold).

FIGS. 57A-B show the nucleotide (SEQ ID NO: 111) and amino acid (SEQ ID NO: 112) sequences, respectively, for a fusion protein including a full-length HPV-16 L2 polypeptide and an RSV G polypeptide consisting of residues 157-168 (shown in bold).

FIGS. 58A-B show the nucleotide (SEQ ID NO: 113) and amino acid (SEQ ID NO: 114) sequences, respectively, for a fusion protein including an N-terminal HPV-16 L2 polypeptide and an RSV G polypeptide consisting of residues 154-167 (shown in bold).

FIGS. 59A-B show the nucleotide (SEQ ID NO: 115) and amino acid (SEQ ID NO: 116) sequences, respectively, for a fusion protein including an N-terminal HPV-16 L2 polypeptide and an RSV G polypeptide consisting of residues 157-168 (shown in bold).

FIG. 60A shows expression of L2N fusion proteins bearing portions of the RSV F protein. Sf9 extracts from cells mock infected (ctrl) or infected with baculovirus designed to express L2N-RSV F fusion proteins (1-4) were resolved on a 12%/6% SDS-PAGE, transferred onto nitrocellulose (NC), and probed with α-FLAG mAb (1:5000) and goat α-mouse HRP antibody (1; 20,000) prior to visualization by ECL (Pierce). Molecular weights (in kD) are shown on the leftmost lane. FIGS. 60B-E show characterizations of cVLPs. MW (in kD) are shown on the left margins of the respective panels. In FIG. 60B, each cVLP preparation (25 μl) was resolved on 10%/5% SDS-PAGE and stained with Coomassie Brilliant Blue R-250. A doublet with a prominent upper band of 57 kD is highlighted. The estimated protein concentrations for cVLP preparations shown in this experiment were 0.1 mg/ml (cVLP 1, 2, and 4) and 0.2 mg/ml (cVLP 3). In FIG. 60C, each cVLP preparation (5 μl/lane) was resolved on 10%/5% SDS-PAGE, transferred onto NC, and probed with a rabbit polyclonal Ab that recognizes denatured HPV serotype 16L1 epitopes (409: 1:20,000) followed by donkey α-rabbit-horseradish peroxidase (HRP) Ab (1:20,000) and ECL. In FIG. 60D, each cVLP preparation (50 μL1/lane) was resolved on 10%/5% SDS-PAGE, transferred onto NC, and probed with α-FLAG mAb (1:5,000) followed by goat α-mouse-HRP (1:20,000) and ECL. The bands corresponding to the expected sizes of L2N-RSV F fusion proteins are marked by arrowheads. In FIG. 60E, immunoblots in which purified RSV F protein (subgroup A; the predominant band in the F protein lanes represents the 50 kD F₁ subunit), L1L2N control VLPs, or cVLPs 3 and 4 were resolved on 10%/5% SDS-PAGE and blotted with α-RSV F mAbs L4 (top panel) or A8 (bottom panel; 1:1000 for each mAb) prior to incubation with goat α-mouse-HRP Ab (1:20,000) and visualization with ECL. Arrowheads indicate the bands consistent with the sizes of the L2N-RSV F fusion proteins that are recognized by each mAb. Bands above and below the highlighted bands are likely due to non-specific recognition since they are found in immunoblots using L4 and A8, and other F-specific and non-F specific mAbs including α-FLAG (see FIG. 60D).

FIG. 61A-C show immunological and morphological characterizations of HPV/RSV cVLPs. FIG. 61A shows the ELISA results in which purified HPV L1 VLPs (100 ng/well) or HPV/RSV cVLP 3 (75 ng/well) were added to 96 well microtiter plates and probed with rabbit polyclonal antibody 079 (recognizes conformation-dependent neutralizing epitopes on HPV16L1 VLPs), 261 (recognizes neutralizing epitopes on heterologous HPV type 18 μl VLPs), or 409 (recognizes denatured 16L1 epitopes). Primary antibodies at 1:100 starting dilutions were serially diluted 1:1. Following incubation with horse α-rabbit IgG-AP conjugated antibody (1:3000) and addition of chromogenic substrate, OD_{405 nm} was read following incubation for 60 minutes at RT. FIG. 61A also shows, below the graph, TEM images of cVLPs 1, 3, and 4 (80,000×). The estimated diameter of the cVLP structures ranged from 60-80 nm, which are somewhat larger than the 55-60 nm diameters of L1 or L1/L2 VLPs; it is possible that the presence of RSV F aa sequence is slightly altering the cVLP structure. Note that these cVLPs were derived in pilot-scale experiments and were relatively dilute (0.1-0.2 mg/ml) with respect to their protein concentrations. The gross morphology of the cVLPs appear very similar to those of wild type L1 or L1L2 HPV VLPs. FIG. 61B shows a TEM image of cVLP 3 generated on a large-scale, high concentration (1.5 mg/ml) preparation. Note the presence of an intact cVLP capsid on the right edge of the image. Otherwise, the majority of the images are consistent with significantly disrupted capsid structures. FIG. 61C shows a TEM image of cVLP 3 (same preparation as in FIG. 61B) that was obtained in the presence of 0.01% Tween-20. Note the distinct capsid structures of ˜55 nm that are consistent with those of HPV VLPs.

FIG. 62A-B shows immunoblot analyses of 16L1 VLPs cross-linked to RSV F-derived peptide. The resulting peptide-linked VLPs were examined using anti-16L1 mAb (FIG. 62A) and anti-F mAb (L4; FIG. 62B).

FIG. 63 shows ELISA analysis of 16L1 VLPs cross-linked to RSV F-derived peptide. L4 anti-RSV F mAb was serially diluted two-fold (starting at 1:5,000) and incubated with 100 ng/well of chemically conjugated 16L1 VLP (solid line) or 1, 2, or 5 ng/well of RSV F-derived peptide (dotted lines). The resulting OD405 nm of the colorimetric reactions are shown. The L4 mAb does not recognize unmodified 16L1 VLPs.

FIG. 64 shows transmission electron micrographs of the 16L1 VLPs chemically cross-linked to RSV F 254-278 peptide. There appear to be two populations of VLPs based on particle size: the smaller (T=1 symmetry group) being approximately 30-35 nm in diameter and larger species (T=7 symmetry group) measuring approximately 55-60 nm across representative particles.

FIG. 65 shows a schematic diagram of a HPV 16L1 pentamer. The interactions among helices 2, 3, and 4 are required for inter-capsomeric interactions and capsid formation. Helix 4 is exposed on the external surface of capsomeres (Bishop et al., “Structure-based Engineering of Papillomavirus Major Capsid 11: Controlling Particle Assembly,” Virol J 4:3 (2007), which is hereby incorporated by reference in its entirety).

FIG. 66 shows schematic ribbon diagrams of 16L1 monomer and two helix 4 deletions. The top panel shows an intact 16L1 monomer and its aa sequence around the h4 domain. The two deletions (aa 404-437 and 410-429) are shown in the lower sections.

FIG. 67 shows a schematic illustration of 16L1 deletion (aa 404-437), and its modified version bearing RSV F aa 255-278. The right panel shows a Coomassie gel of the initial efforts to purify capsomere derivatives; in subsequent preparations, the predominant 55 kD L1 derivatives are deemed >80-85% pure.

FIG. 68 shows immunoblot analyses of L1 capsomere derivatives. The top panel shows that purified L1 derivatives are recognized by an anti-16L1 mouse mAb (1:40,000 dilution), while the bottom panel shows that the L1 derivative bearing RSV F aa 255-278 is recognized by the L4 anti-RSV F mAb (1:5,000 dilution). Relevant positive controls (purified wild type 16L1 and RSV F proteins) are also shown.

FIG. 69 shows ELISA analysis of purified L1 capsomere derivatives. Preimmune mouse sera or immune sera against purified 16L1 VLP preparations were serially diluted two-fold (starting at 1:200) and incubated with 50-100 ng/well of purified 16L1 VLP or either of the L1 capsomere derivatives. The resulting OD405 nm of the colorimetric reactions are shown. One series (solid line) shows that the L1del 1+RSV F 255-278 is recognized by the anti-RSV F neutralizing mAb (L4).

FIG. 70 shows a representative ELISA assay in which 50-100 ng/well of purified RSV F protein or either L1 capsomere derivative were incubated with serially diluted anti-F polyclonal or monoclonal antibodies (starting dilution at 1:200). The resulting OD405 nm were plotted as above. The 16L1 del1 alone is not recognized by anti-RSV antibodies whereas its derivative bearing RSV F aa 255-278 and purified F protein are recognized by anti-F antibodies.

FIG. 71 shows transmission electron micrographs of 16L1 VLP that is fragmented into capsomeres (left panel) and 16L1 capsomere derivative (del1) bearing RSV F aa 255-278 (right panel). Note the circular, ring-like morphology of the capsomeres comprising 16L1 VLPs. Similar ring-like structures are also seen in capsomere derivatives. The focally aggregated distribution of the capsomeres is noted; we suspect that this may be an artifact of TEM sample preparation, and sucrose gradient centrifugation analyses of the capsomere preparations are being performed to confirm that the capsomeres exist in monomeric (instead of aggregated, oligomeric forms) in solution.

FIG. 72 shows analyses of week 10 (terminal) bleeds of mice injected with capsomere derivatives bearing F moieties. Shown are immunoblots in which pooled Week 10 sera (1:1,000 dilution) from 3-4 BALB/c mice injected with various capsomere derivatives were assayed for their ability to recognize purified RSV F protein (approximately 2 μg/lane) under denaturing conditions and visualized using anti-mouse-HRP conjugated secondary antibody (1:20,000) and chemiluminescence kit (ECL; Pierce).

FIG. 73A-C shows representative ELISA assays to characterize week 10 bleeds from capsomere-injected mice (see FIG. 72 legends for immunization details). In FIG. 73A, 50-100 ng/well of purified RSV G protein was incubated with serially diluted anti-G mAb (L9; starting dilution 1:5,000) or pooled sera from mice injected with 16L1del2, 16L1del2+RSV G aa 154-167, or 16L1del2+RSV G aa 157-168 (starting dilutions at 1:200). The resulting OD405 nm were plotted as above. In FIG. 73B, 100 ng/well of peptide cross-linked to 16L1 VLPs (RSV F aa 254-278 bearing the amino-terminal linker sequence CGG preceding the F-derived aa) was incubated with serially diluted anti-F mAb (L4; starting dilution 1:5,000) or pooled sera from mice injected with 16L1del2, 16L1del1+RSV F aa 423-436, 16L1del2+RSV F aa 423-436, or 16L1del1+RSV F aa 255-278 (starting dilutions at 1:200). The resulting OD405 nm were plotted as above. In FIG. 73C, 100 ng/well of purified RSV F protein was incubated with serially diluted anti-F mAb (L4; starting dilution 1:5,000) or pooled sera from mice injected with 16L1del2, 16L1del1+RSV F aa 423-436, 16L1del2+RSV F aa 423-436, or 16L1del1+RSV F aa 255-278 (starting dilutions at 1:200). The resulting OD405 nm were plotted as above.

### DETAILED DESCRIPTION OF THE INVENTION

The present invention relates to the production of chimeric papillomavirus virus-like particles (VLPs) or capsomeres that include one or more respiratory syncytial virus (RSV) polypeptides, and use thereof as a vaccine platform against RSV.

Papillomaviruses are small, double-stranded, circular DNA tumor viruses. The papillomavirus virion shells contain the L1 major capsid protein and the L2 minor capsid protein. Expression of L1 protein alone or in combination with L2 protein in eukaryotic or prokaryotic expression systems is known to result in the assembly of capsomeres and VLPs.

As used herein, the term “capsomere” is intended to mean a pentameric assembly of papillomavirus L1-containing polypeptides (including full-length L1 protein and fragments thereof) or L1-containing fusion polypeptides. Native L1 capsid proteins self-assemble via intermolecular disulfide bonds to form pentamers (capsomeres). It has been shown previously that L1 capsomeres induce serotype-specific neutralizing antibodies in mice, induce L1-specific CTL responses and tumor regression in mice, and that the vast majority of surface-exposed anti-HPV antibody epitopes are located on the capsomere loops (Rose et al., “Human Papillomavirus Type 11 Recombinant L1 Capsomeres Induce Virus-Neutralizing Antibodies,” J Virol 72:6151-6154 (1998); Ohlschlager et al., “Human Papillomavirus Type 16 L1 Capsomeres Induce L1-specific Cytotoxic T Lymphocytes and Tumor Regression in C57BL/6 Mice,” J Virol 77: 4635-4645 (2003); and Yuan et al., “Immunization with a Pentameric L1 Fusion Protein Protects against Papillomavirus Infection,” J Virol 75: 7843-7853 (2001), each of which is hereby incorporated by reference in its entirety. Taken together, capsomeres have the potential as a vaccine platform to elicit a broad range of cellular and humoral immune responses.

As used herein, the term “virus-like particle” or VLP is intended to mean a particle comprised of a higher order assembly of capsomeres. VLPs are non-infectious and non-replicating, yet morphologically similar to native papillomavirus virion. One example of such a higher order assembly is a particle that has the visual appearance of a whole (72 capsomere) or substantially whole, empty papillomavirus capsid, which is about 50 to about 60 nm in diameter and has a T=7 icosahedral construction. Another example of such a higher order assembly is a particle of about 30 to about 35 nm in diameter, which is smaller than the size of a native papillomavirus virion and has a T=1 construction (containing 12 capsomeres). For purposes of the present invention, other higher order assemblies of capsomeres are also intended to be encompassed by the term VLP. The VLPs and capsomeres preferably, but need not, replicate conformational epitopes of the native papillomavirus from which the L1 protein or polypeptide or L2 protein or polypeptide is derived. Methods for assembly and formation of human papillomavirus VLPs and capsomeres of the present invention are well known in the art (U.S. Pat. No. 6,153,201 to Rose et al.; U.S. Pat. No. 6,165,471 to Rose et al.; WO 94/020137 to Rose et al., each of which is hereby incorporated by reference in its entirety).

As used herein, the term “chimeric” is intended to denote VLPs or capsomeres that include polypeptide components from two or more distinct sources, e.g., a papillomavirus and an RSV. This term is not intended to confer any meaning concerning the specific manner in which the polypeptide components are bound or attached together.

Preferably, the chimeric papillomavirus VLP or capsomere includes an L1 polypeptide and, optionally, an L2 polypeptide, and a respiratory syncytial virus (RSV) protein or polypeptide fragment thereof that includes a first epitope, where the RSV protein or polypeptide fragment thereof is attached to one or both of the L1 and L2 polypeptides.

The L1 polypeptide can be full-length L1 protein or an L1 polypeptide fragment. According to one embodiment, the full-length L1 protein or L1 polypeptide fragment can be VLP assembly-competent (that is, the L1 polypeptide will self-assemble to form capsomeres that are competent for self-assembly into a higher order assemblies, thereby forming a VLP). According to another embodiment, the full-length L1 protein or L1 polypeptide fragment can be VLP assembly-incompetent (that is, the L1 polypeptide will form capsomeres that are unable to assemble into higher order assemblies of a VLP). L1 polypeptides that lack at least a portion of the helix 4 (“h4”) domain, preferably the entire h4 domain (residues 412-428 of HPV-16 L1) and its surrounding amino acids, also lack the ability to form L1 VLPs, but the resulting L1 derivatives are capable of self-assembly into capsomeres (Bishop et al., “Structure-based Engineering of Papillomavirus Major Capsid L1: Controlling Particle Assembly,” Virol J 4:3, pp. 1-6 (2007), which is hereby incorporated by reference in its entirety).

The L1 sequences are known for substantially all papillomavirus genotypes identified to date, and any of these L1 sequences or fragments can be employed in the present invention. Examples of L1 polypeptides include, without limitation, full-length L1 polypeptides, L1 truncations that lack the native C-terminus, L1 truncations that lack the native N-terminus, and L1 truncations that lack an internal domain. As described hereinafter, L1 fusion proteins can include the heterologous, RSV polypeptide linked at the N-terminus of the L1 polypeptide, the C-terminus of the L1 polypeptide, or at internal sites of the L1 polypeptide, including where portions of the native L1 sequence have been deleted.

The L2 polypeptide can be full-length L2 protein or an L2 polypeptide fragment. The L2 sequences are known for substantially all papillomavirus genotypes identified to date, and any of these L2 sequences or fragments can be employed in the present invention. Examples of L2 polypeptides include, without limitation, full-length L2 polypeptides, L2 truncations that lack the native C-terminus, L2 truncations that lack the native N-terminus, and L2 truncations that lack an internal domain. As described hereinafter, L2 fusion proteins can include the heterologous, RSV polypeptide linked at the N-terminus of the L2 polypeptide, the C-terminus of the L2 polypeptide, or at internal sites of the L2 polypeptide, including where portions of the native L2 sequence have been deleted.

The chimeric papillomavirus VLPs and capsomeres can be formed using the L1 and optionally L2 polypeptides from any animal papillomavirus, or derivatives or fragments thereof. Thus, any known (or hereafter identified) L1 and optional L2 sequences of human, bovine, equine, ovine, porcine, deer, canine, feline, rodent, rabbit, etc., papillomaviruses can be employed to prepare the VLPs or capsomeres of the present invention.

In one embodiment of the present invention, the L1 and optionally L2 polypeptides of the papillomavirus VLP are derived from human papillomaviruses. Preferably, they are derived from HPV-6, HPV-11, HPV-16, HPV-18, HPV-31, HPV-33, HPV-35, HPV-39, HPV-45, HPV-52, HPV-54, HPV-58, HPV-59, HPV-64, or HPV-68. For a near complete listing of papillomavirus genotypes and their relatedness, see de Villiers et al., “Classification of Papillomaviruses,” Virology 324:17-27 (2004), which is hereby incorporated by reference in its entirety. The L1 and L2 sequences are known for substantially all papillomaviruses identified to date, e.g., HPV-18 (Genbank accessions NC_{—}001357 and X05015, which are hereby incorporated by reference in its entirety); HPV-64 (NC_{—}001676 and U37488, which are hereby incorporated by reference in its entirety); and all other HPV genotypes. Exemplary genital-specific genotypes of human papillomavirus include, but are not limited to HPV-6, -11, -16, -18, -30, -31, -33, -34, -35, -39, -60, -62, -43, -64, -65, -51, -52, -53, -54, -56, -58, -59, -61, -62, -66, -67, -68, -69, -70, -71, -74, -81, -85, -86, -87, -89, -90, -91, -92, -101, -102, -103, and -106. Some of the genital-specific genotype human papillomaviruses are associated with cancer, including HPV-16, -18, -31, -33, -35, -39, -45, -51, -52, -56, -58, -59, -66, -67, -68, -73, and -82. Exemplary nongenital-specific genotypes of human papillomavirus include, but are not limited to, HPV-1, -2, -3, -4, -7, -10, -22, -28, -29, -36, -37, -38, -41, -48, -49, -60, -63, -67, -72, -76, -77, -80, -88, -92, -93, -94, -98, -95, -96, and -107. VLPs or capsomeres of other HPV genotypes, whether newly discovered or previously known, can also be used.

According to one embodiment, the L1 and optionally L2 polypeptides that are used to form the VLPs or capsomeres are from a non-human papillomavirus or a human papillomavirus genotype other than HPV-6, HPV-11, HPV-16, and HPV-18. This embodiment may be commercially desirable, because it may avoid the possibility of inducing immune tolerance against any HPV genotypes that are utilized in commercial HPV vaccines. To the extent that commercial vaccine formulations are altered, then it is contemplated to utilize L1 and optionally L2 polypeptides derived from human papillomaviruses other than those presented in such vaccine formulations.

RSV is an enveloped virus of the Paramyxoviridae family (Collins et al., “Nucleotide Sequences for the Gene Junctions of Human Respiratory Syncytial Virus Reveal Distinctive Features of Intergenic Structure and Gene Order,” Proc Natl Acad Sci USA 83:4594-4598 (1986); Collins et al., “Rational Design of Live-attenuated Recombinant Vaccine Virus for Human Respiratory Syncytial Virus by Reverse Genetics,” Adv Virus Res 54:423-451 (1999), each of which is hereby incorporated by reference in its entirety). RSV isolates are broadly classified into one of two antigenic groups, A or B (Anderson et al., “Antigenic Characterization of Respiratory Syncytial Virus Strains with Monoclonal Antibodies,” J Infect Dis 151:626-633 (1985); Cristina et al., “Analysis of Genetic Variability in Human Respiratory Syncytial Virus by the RNase A Mismatch Cleavage Method: Subtype Divergence and Heterogeneity,” Virology 174:126-134 (1990); Sullender, “Respiratory Syncytial Virus Genetic and Antigenic Diversity,” Clin Microbiol Rev 13:1-15 (2000), each of which is hereby incorporated by reference in its entirety). Each virion contains a non-segmented, (−) single-stranded RNA that encodes eight structural and three non-structural (NS1, NS2, M2-2) proteins (Collins et al., “Nucleotide Sequences for the Gene Junctions of Human Respiratory Syncytial Virus Reveal Distinctive Features of Intergenic Structure and Gene Order,” Proc Natl Acad Sci USA 83:4594-4598 (1986); Dickens et al., “Transcriptional Mapping of Human Respiratory Syncytial Virus,” J Virol 52:364-369 (1984); Wertz et al., “Nucleotide Sequence of the G Protein Gene of Human Respiratory Syncytial Virus Reveals an Unusual Type of Viral Membrane Protein,” Proc Natl Acad Sci USA 82:4075-4079 (1985), each of which is hereby incorporated by reference in its entirety). The viral envelope bears three transmembrane glycoproteins (G, F, SH) as well as the matrix (M) protein (Collins et al., “cDNA Cloning and Transcriptional Mapping of Nine Polyadenylated RNAs Encoded by the Genome of Human Respiratory Syncytial Virus,” Proc Natl Acad Sci USA 80:3208-3212 (1983); Collins et al., “The 1A Protein Gene of Human Respiratory Syncytial Virus: Nucleotide Sequence of the mRNA and a Related Polycistronic Transcript,” Virology 141:283-291 (1985); Collins et al., “The Envelope-associated 22K Protein of Human Respiratory Syncytial Virus: Nucleotide Sequence of the mRNA and a Related Polytranscript,” J Virol 54:65-71 (1985); Collins et al., “Nucleotide Sequences of the 1B and 1C Nonstructural Protein mRNAs of Human Respiratory Syncytial Virus,” Virology 143:442-451 (1985); Collins et al., “Gene Overlap and Site-specific Attenuation of Transcription of the Viral Polymerase L Gene of Human Respiratory Syncytial Virus,” Proc Natl Acad Sci USA 84:5134-5138 (1987), each of which is hereby incorporated by reference in its entirety). Within the envelope, viral RNA is encapsidated by a transcriptase complex comprised of the N (nucleocapsid), P (phosphoprotein), M2-1 (transcription elongation factor), and L (polymerase) proteins (Collins et al., “Gene Overlap and Site-specific Attenuation of Transcription of the Viral Polymerase L Gene of Human Respiratory Syncytial Virus,” Proc Natl Acad Sci USA 84:5134-5138 (1987), which is hereby incorporated by reference in its entirety). Among viral isolates, some RSV-encoded proteins such as F are highly conserved with respect to amino acid sequence while others such as G display extensive antigenic variation between and within the two major antigenic groups (Johnson et al., “The Fusion Glycoproteins of Human Respiratory Syncytial Virus of Subgroups A and B: Sequence Conservation Provides a Structural Basis for Antigenic Relatedness,” J Gen Virol 69 (Pt 10):2623-2628 (1988); Johnson et al., “The G Glycoprotein of Human Respiratory Syncytial Viruses of Subgroups A and B: Extensive Sequence Divergence Between Antigenically Related Proteins,” Proc Natl Acad Sci USA 84:5625-5629 (1987); Garcia-Barreno et al., “Marked Differences in the Antigenic Structure of Human Respiratory Syncytial Virus F and G Glycoproteins,” J Virol 63:925-932 (1989), each of which is hereby incorporated by reference in its entirety).

The various RSV-encoded proteins have been extensively analyzed with respect to their immunogenicity. Several proteins, including N, M2-1, NS1, and F, bear epitopes that induce CTL responses in murine- and/or human-derived lymphocytes (Rutigliano et al., “Identification of an H-2D(b)-restricted CD8+ Cytotoxic T Lymphocyte Epitope in the Matrix Protein of Respiratory Syncytial Virus,” Virology 337:335-343 (2005); Pemberton et al., “Cytotoxic T Cell Specificity for Respiratory Syncytial Virus Proteins: Fusion Protein is an Important Target Antigen,” J Gen Virol 68 (Pt 8):2177-2182 (1987); Openshaw et al., “The 22,000-kilodalton Protein of Respiratory Syncytial Virus is a Major Target for kd-restricted Cytotoxic T Lymphocytes from Mice Primed by Infection,” J Virol 64:1683-1689 (1990), each of which is hereby incorporated by reference in its entirety). In contrast, extensive efforts to identify CTL epitopes within others, including the G protein, have been unfruitful (Bukreyev et al., “The Cysteine-rich Region and Secreted Form of the Attachment G Glycoprotein of Respiratory Syncytial Virus Enhance the Cytotoxic T-lymphocyte Response Despite Lacking Major Histocompatibility Complex Class I-restricted Epitopes,” J Virol 80:5854-5861 (2006), which is hereby incorporated by reference in its entirety). With regard to humoral response, only antibodies against F or G are neutralizing and confer resistance to RSV upon passive transfer in animal models (Murphy et al., “Passive Transfer of Respiratory Syncytial Virus (RSV) Antiserum Suppresses the Immune Response to the RSV Fusion (F) and Large (G) Glycoproteins Expressed by Recombinant Vaccinia Viruses,” J Virol 62:3907-3910 (1988); Barbas et al., “Human Monoclonal Fab Fragments Derived from a Combinatorial Library Bind to Respiratory Syncytial Virus F Glycoprotein and Neutralize Infectivity,” Proc Natl Acad Sci USA 89:10164-10168 (1992); Walsh et al., “Protection from Respiratory Syncytial Virus Infection in Cotton Rats by Passive Transfer of Monoclonal Antibodies,” Infect Immun 43:756-758 (1984); Taylor et al., “Monoclonal Antibodies Protect Against Respiratory Syncytial Virus,” Lancet 2:976 (1983); Taylor et al., “Monoclonal Antibodies Protect Against Respiratory Syncytial Virus Infection in Mice,” Immunology 52:137-142 (1984), each of which is hereby incorporated by reference in its entirety). A number of F-specific neutralizing mAbs also possess the ability to inhibit viral fusion activity (Arbiza et al., “Characterization of Two Antigenic Sites Recognized by Neutralizing Monoclonal Antibodies Directed Against the Fusion Glycoprotein of Human Respiratory Syncytial Virus,” J Gen Virol 73 (Pt 9):2225-2234 (1992); Barbas et al., “Human Monoclonal Fab Fragments Derived From a Combinatorial Library Bind to Respiratory Syncytial Virus F Glycoprotein and Neutralize Infectivity,” Proc Natl Acad Sci USA 89:10164-10168 (1992); Beeler et al., “Neutralization Epitopes of the F Glycoprotein of Respiratory Syncytial Virus Effect of Mutation Upon Fusion Function,” J Virol 63:2941-2950 (1989); Walsh et al., “Monoclonal Antibodies to Respiratory Syncytial Virus Proteins: Identification of the Fusion Protein,” J Virol 47:171-177 (1983), each of which is hereby incorporated by reference in its entirety). Thus, among RSV-encoded proteins, the F protein is unique in that it has the potential to elicit cellular and humoral responses, and the protective effect and the clinically significant protective effect of F-specific neutralizing antibodies has been validated.

According to the present invention, the RSV polypeptide can be derived from NS1, NS2, N, P, M, M2, L, SH, F, and G proteins, or any combination thereof, but preferably the F and G proteins, or a combination thereof. These RSV polypeptides can be derived from either a group A RSV or a group B RSV. The one or more RSV proteins or polypeptide fragments thereof include a first epitope, which is preferably one that is capable of inducing a neutralizing antibody response against RSV, generating a Th1-associated CTL response, and a Th1-dominant immune response that avoids the pulmonary pathology associated with Th2 response.

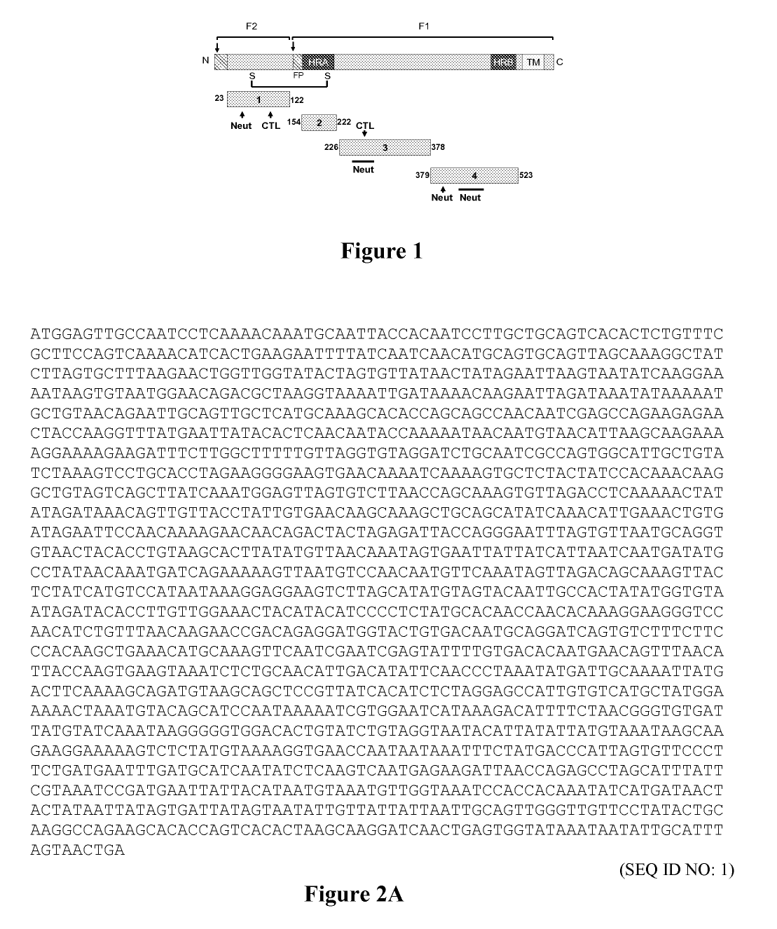

The salient structural and immunological aspects of the F protein are shown in FIG. 1. The nascent Fₒ protein is cleaved by intracellular proteases to generate two subunits, F1 (˜50 kD) and F2 (˜20 kD), that are covalently linked by a disulfide bond (Collins et al., “Post-Translational Processing and Oligomerization of the Fusion Glycoprotein of Human Respiratory Syncytial Virus,” J Gen Virol. 72(12):3095-3101 (1991), which is hereby incorporated by reference in its entirety). The F1 subunit contains three structural motifs: heptad repeats A (HRA) and B (HRB), which are involved in conformational changes of F protein during membrane fusion, and the membrane anchoring transmembrane (TM) domain (Branigan et al., “The Cytoplasmic Domain of the F Protein of Human Respiratory Syncytial Virus is not Required for Cell Fusion,” J Gen Virol. 87:395-398 (2006); Yin et al., “Structure of the Parainfluenza Virus 5F Protein in its Metastable, Prefusion Conformation,” Nature 439:38-44 (2006), each of which is hereby incorporated by reference in its entirety). In the native conformation, the F protein exists as a homomeric trimer (Collins et al., “Post-Translational Processing and Oligomerization of the Fusion Glycoprotein of Human Respiratory Syncytial Virus,” J Gen Virol. 72(12):3095-3101 (1991), which is hereby incorporated by reference in its entirety). Based primarily on analysis of mutant viruses resistant to mAbs, neutralizing epitopes within the F protein have been mapped to amino acid residues 32 (in conjunction with residue 272), 237, 241 (in conjunction with residue 421), 255-275, 389, and 429-447 (Beeler et al., “Neutralization Epitopes of the F Glycoprotein of Respiratory Syncytial Virus: Effect of Mutation upon Fusion Function,” J Virol. 63:2941-2950 (1989); Garcia et al., “Mapping of Monoclonal Antibody Epitopes of the Human Respiratory Syncytial Virus P Protein,” Virology 195:239-242 (1993); Lopez et al., “Location of a Highly Conserved Neutralizing Epitope in the F Glycoprotein of Human Respiratory Syncytial Virus,” J Virol. 64:927-930 (1990); Lopez et al., “Antigenic Structure of Human Respiratory Syncytial Virus Fusion Glycoprotein,” J Virol. 72:6922-6928 (1998), each of which is hereby incorporated by reference in its entirety). Of note, amino acid residues 260-275 have been shown to be involved in the binding of the neutralizing monoclonal antibody palivizumab (Zhao et al., “In vivo Selection of Respiratory Syncytial Viruses Resistant to palivizumab,” J Virol. 79:3962-3968 (2005), which is hereby incorporated by reference in its entirety). The F protein also bears epitopes at amino acid residues 85-93, 92-106, and 249-258 that induce H-2K^{d}-restricted CTL responses in mice, and others at amino acid residues 109-118, 118-126, and 551-559 that induce HLA-restricted CTL responses from human-derived peripheral lymphocytes (Rock et al., “Identification of a Novel Human Leucocyte Antigen-A*01-restricted Cytotoxic T-Lymphocyte Epitope in the Respiratory Syncytial Virus Fusion Protein,” Immunology 108:474-480 (2003); Johnstone et al., “Shifting Immunodominance Pattern of Two Cytotoxic T-lymphocyte Epitopes in the F Glycoprotein of the Long Strain of Respiratory Syncytial Virus,” J Gen Virol. 85:3229-3238 (2004); Brandenburg et al., “HLA Class I-restricted Cytotoxic T-cell Epitopes of the Respiratory Syncytial Virus Fusion Protein,” J Virol. 74:10240-10244 (2000); Heidema et al., “Human CD8(+) T Cell Responses Against Five Newly Identified Respiratory Syncytial Virus-derived Epitopes,”J Gen Virol. 85:2365-2374 (2004); Jiang et al., “Virus-specific CTL Responses Induced by an H-2 K^{d}-restricted, Motif-negative 15-mer Peptide from the Fusion Protein of Respiratory Syncytial Virus,” J Gen Virol. 83:429-438 (2002); Chang et al., “Visualization and Characterization of Respiratory Syncytial Virus F-specific CD8(+) T Cells During Experimental Virus Infection,” J Immunol. 167:4254-4260 (2001); Goulder et al., “Characterization of a Novel Respiratory Syncytial Virus-specific Human Cytotoxic T-lymphocyte Epitope,” J Virol. 74:7694-7697 (2000), each of which is hereby incorporated by reference in its entirety).

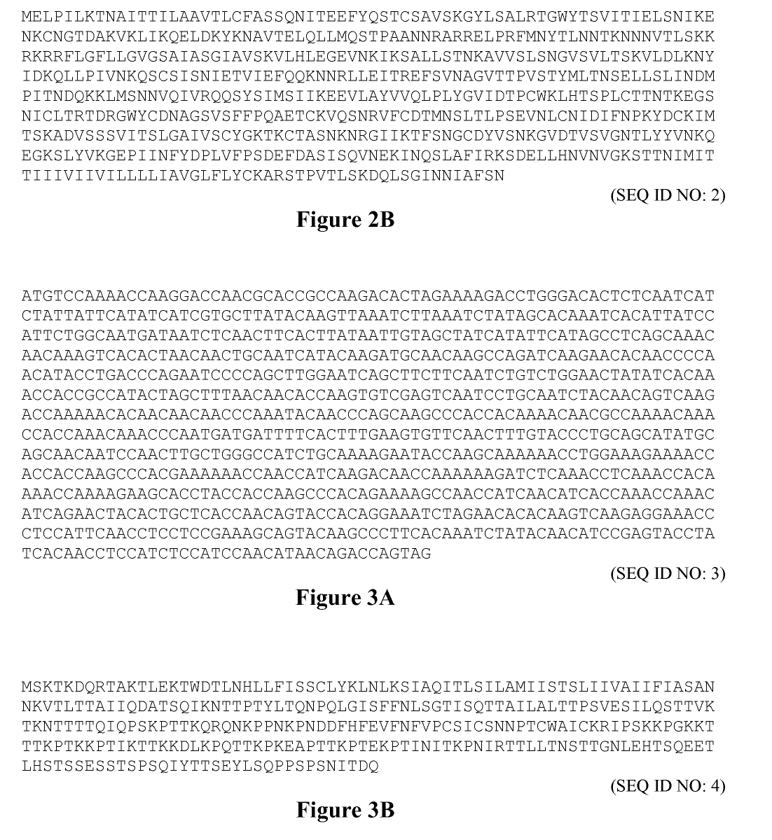

A number of RSV F proteins and their encoding nucleic acids are known in the art including, without limitation, those identified at Genbank Accession Nos. NC_{—}001781, NC_{—}001803, AY114151, Y114150, AY114149, L25351, U31560, U31561, U31562, U31558, U31559, and DQ885231, each of which is hereby incorporated by reference in its entirety. The amino acid sequence of one exemplary F protein, from the RSV RGH strain, and its encoding nucleotide sequence are illustrated in FIGS. 2B (SEQ ID NO: 2) and 2A (SEQ ID NO: 1), respectively.

Exemplary polypeptide fragments of the F protein include, without limitation, polypeptides including (or, in some embodiments, consisting of) amino acid residues 23-122 of SEQ ID NO: 2 (encoded by nt 67-366 of SEQ ID NO: 1), amino acid residues 154-222 of SEQ ID NO: 2 (encoded by nt 460-666 of SEQ ID NO: 1), amino acid residues 226-378 of SEQ ID NO: 2 (encoded by nt 676-1134 of SEQ ID NO: 1), amino acid residues 379-523 of SEQ ID NO: 2 (encoded by nt 1135-1569 of SEQ ID NO: 1), amino acid residues 379-559 of SEQ ID NO: 2 (encoded by nt 1135-1677 of SEQ ID NO: 1), amino acid residues 249-275 of SEQ ID NO: 2 (encoded by nt 745-825 of SEQ ID NO: 1), amino acid residues 254-278 of SEQ ID NO: 2 (encoded by nt 760-834 of SEQ ID NO: 1), amino acid residues 255-278 of SEQ ID NO: 2 (encoded by nt 763-834 of SEQ ID NO: 1), amino acid residues 423-436 of SEQ ID NO: 2 (encoded by nt 1267-1308 of SEQ ID NO: 1), and one or more combinations thereof.

A number of RSV G proteins and their encoding nucleic acids are known in the art including, without limitation, those identified at Genbank Accession Nos. DQ227363, DQ227364, DQ227365, DQ227366, DQ227367, DQ227368, DQ227369, DQ227370, DQ227371, DQ227372, DQ227373, DQ227374, DQ227375, DQ227376, DQ227377, DQ227378, DQ227379, DQ227380, DQ227381, DQ227382, DQ227383, DQ227384, DQ227385, DQ227386, DQ227387, DQ227388, DQ227389, DQ227390, DQ227391, DQ227392, DQ227393, DQ227394, DQ227395, DQ227396, DQ227397, AB117522, AF516119, AY114151, AY114150, AY114149, AY333361, AY333362, AY333363, AY333364, AF065405, and AF065406, each of which is hereby incorporated by reference in its entirety. The amino acid sequence of one exemplary G protein, from the RSV RGH strain, and its encoding nucleotide sequence are illustrated in FIGS. 3B (SEQ ID NO: 4) and 3A (SEQ ID NO: 3).

Exemplary polypeptide fragments of the G protein include, without limitation, polypeptides including (or, in some embodiments, consisting of) amino acid residues 154-167 of SEQ ID NO: 4 (encoded by nt 460-501 of SEQ ID NO: 3), amino acid residues 157-168 of SEQ ID NO: 4 (encoded by nt 469-504 of SEQ ID NO: 3), and combinations thereof.

According to one embodiment of the present invention, the RSV protein or polypeptide fragment is attached via an in-frame gene fusion to one or both of the L1 and L2 polypeptides such that recombinant expression of the L1 and/or L2 fusion proteins results in incorporation of the RSV protein or polypeptide into the self-assembled capsomere or VLPs of the present invention (i.e., with the epitopes thereof available for inducing the elicitation of a high-titer neutralizing antibody response).

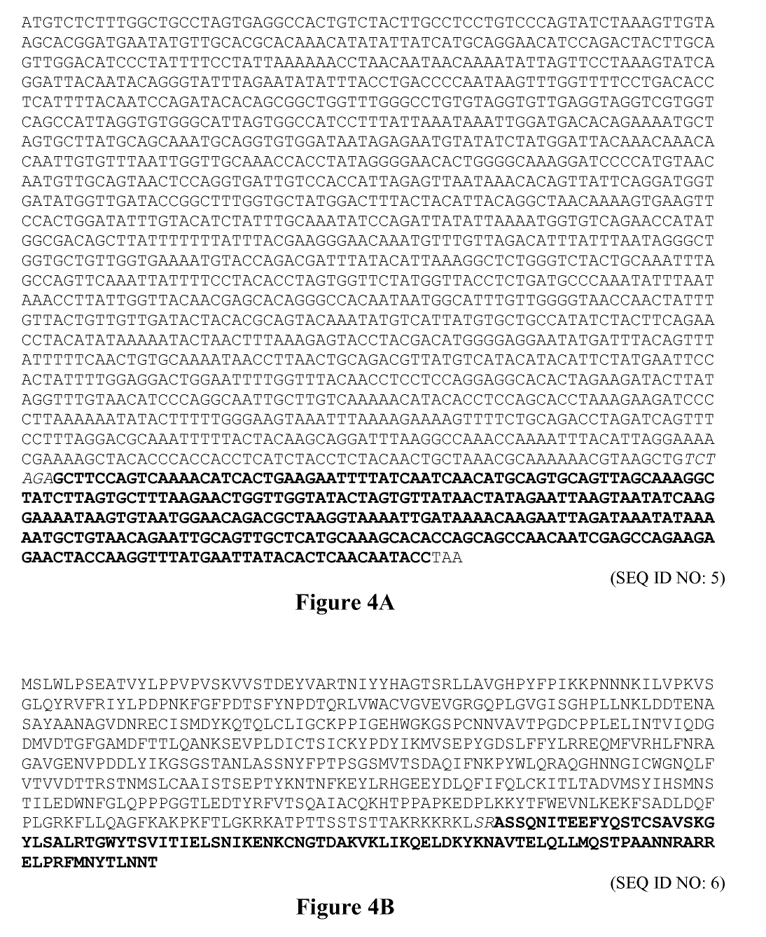

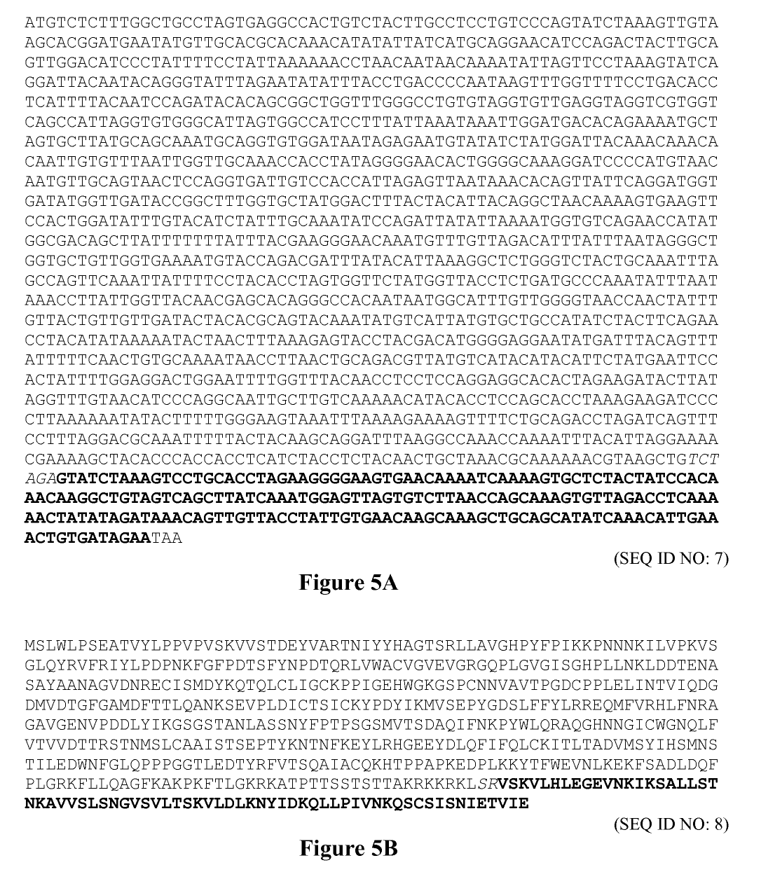

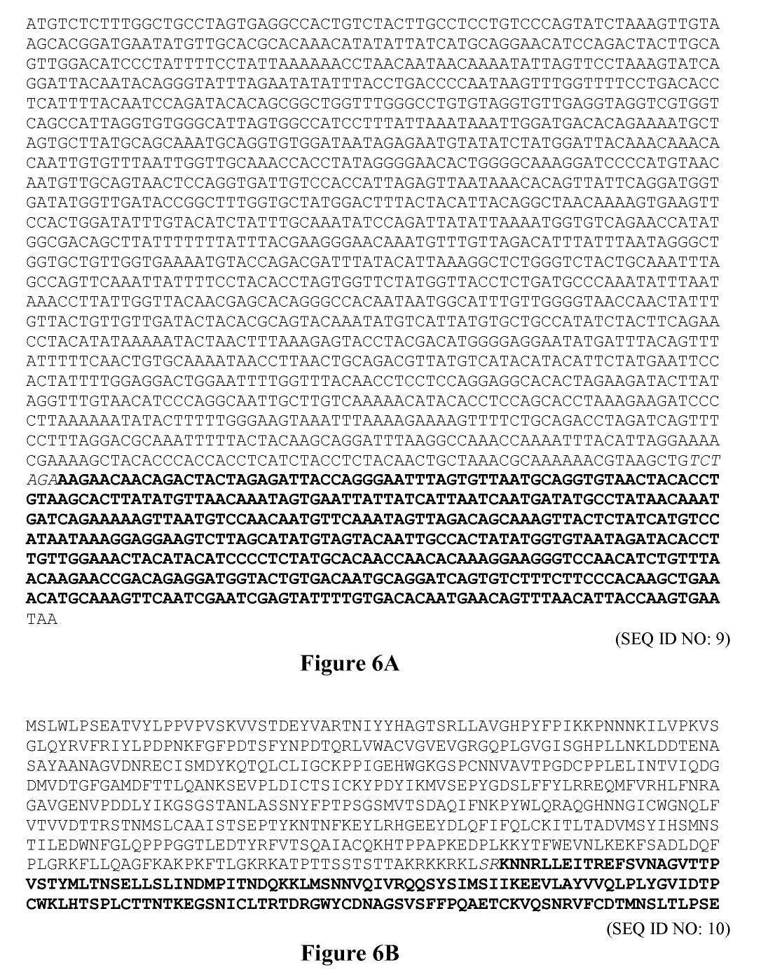

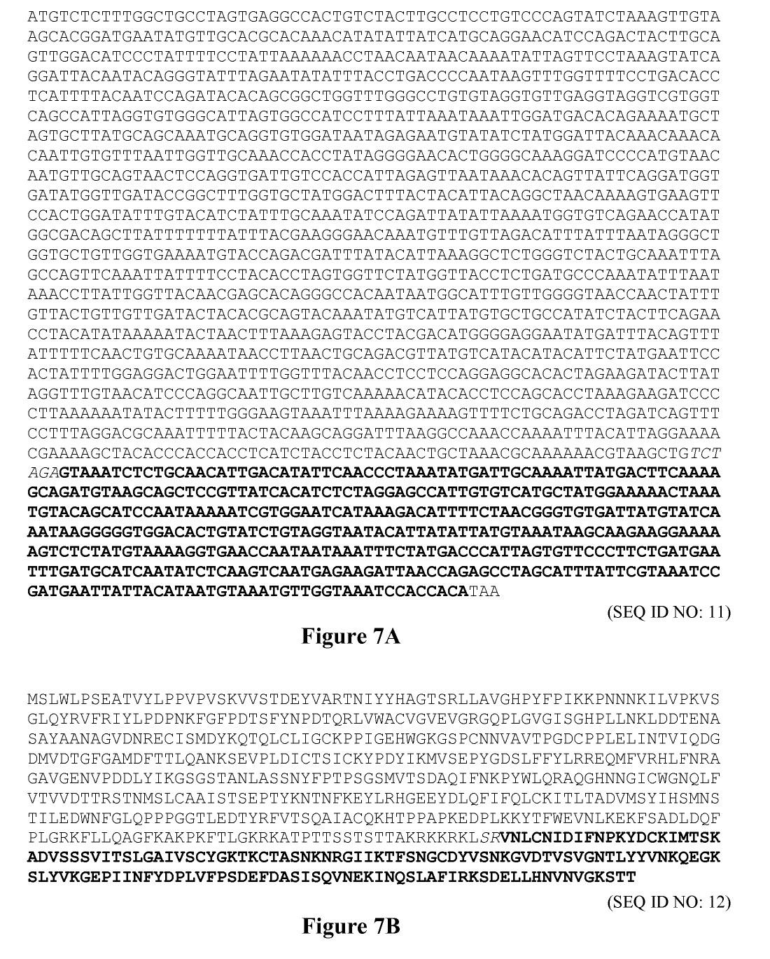

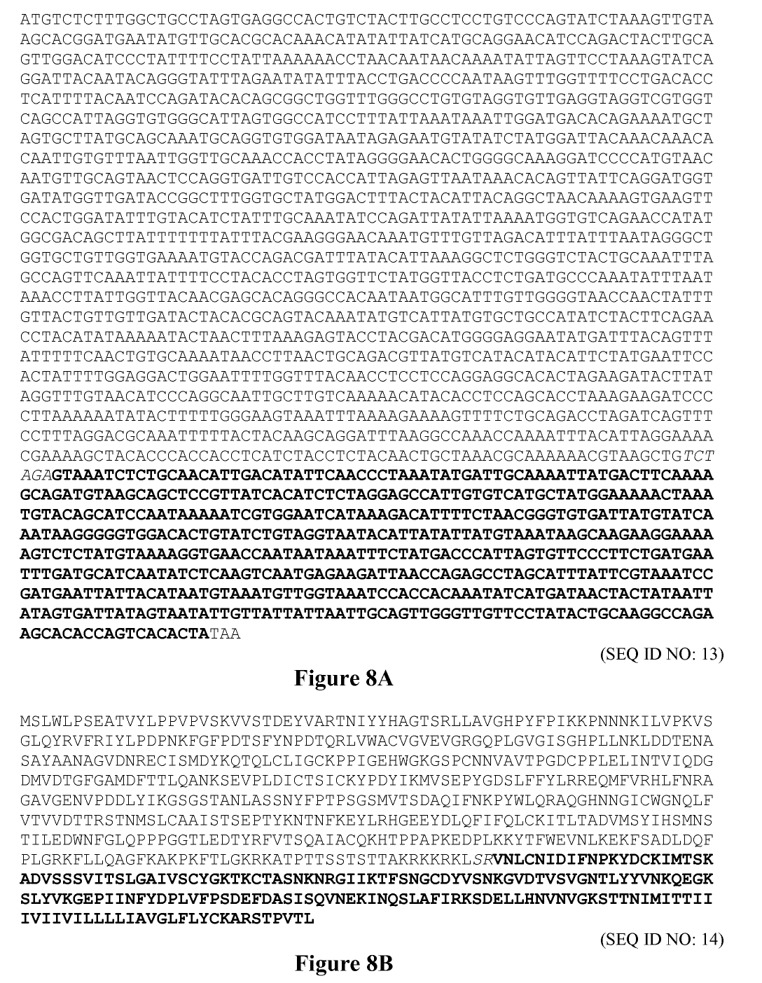

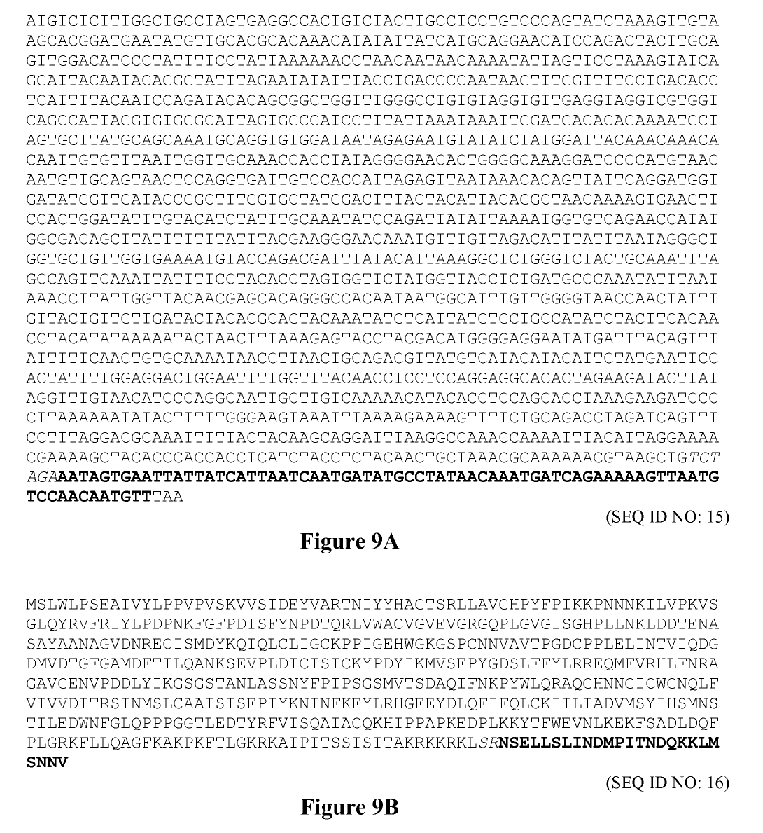

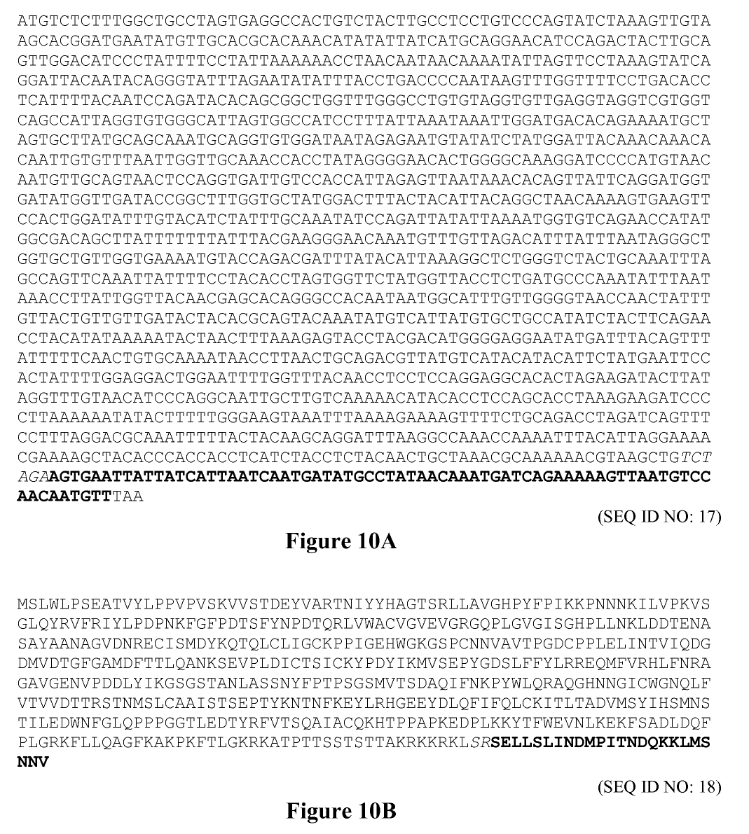

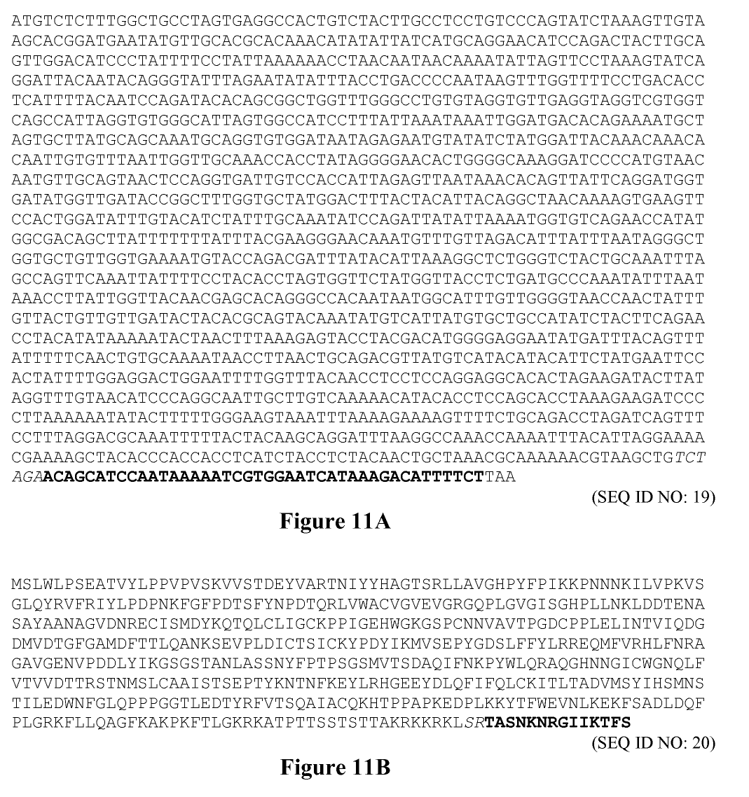

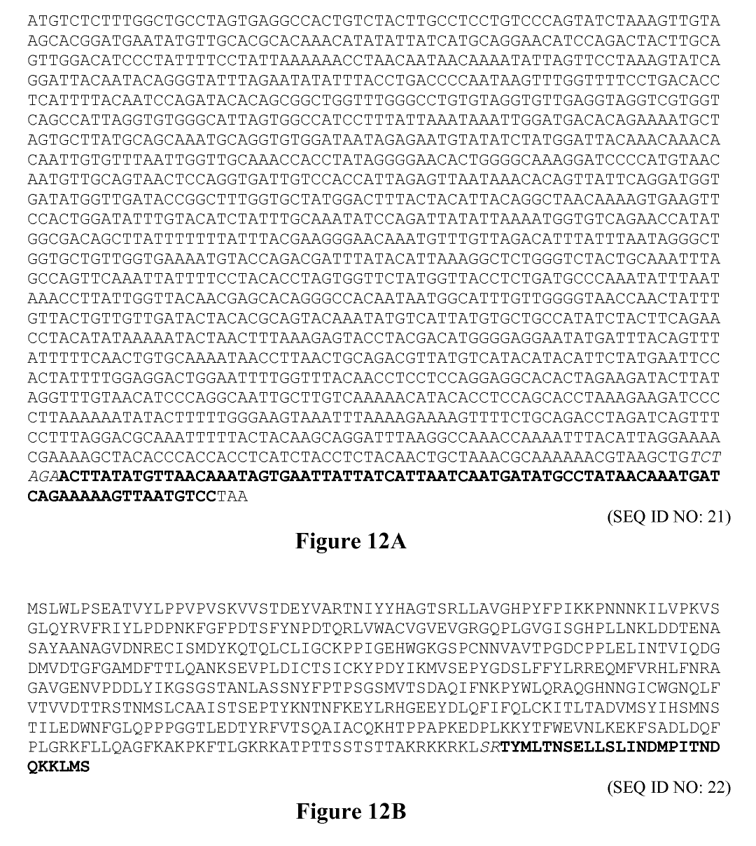

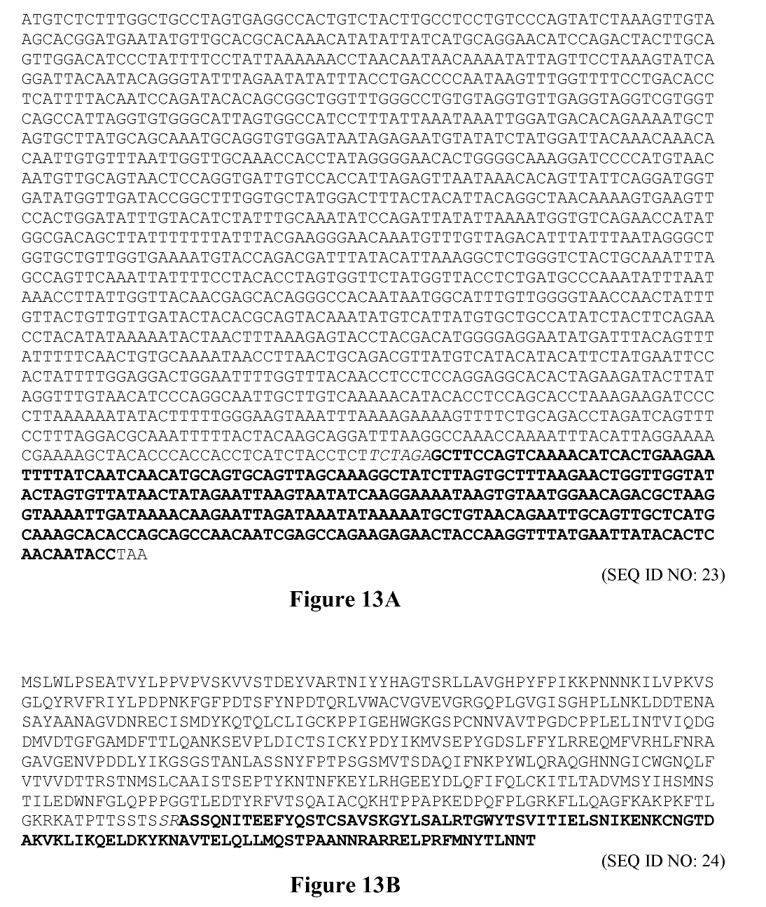

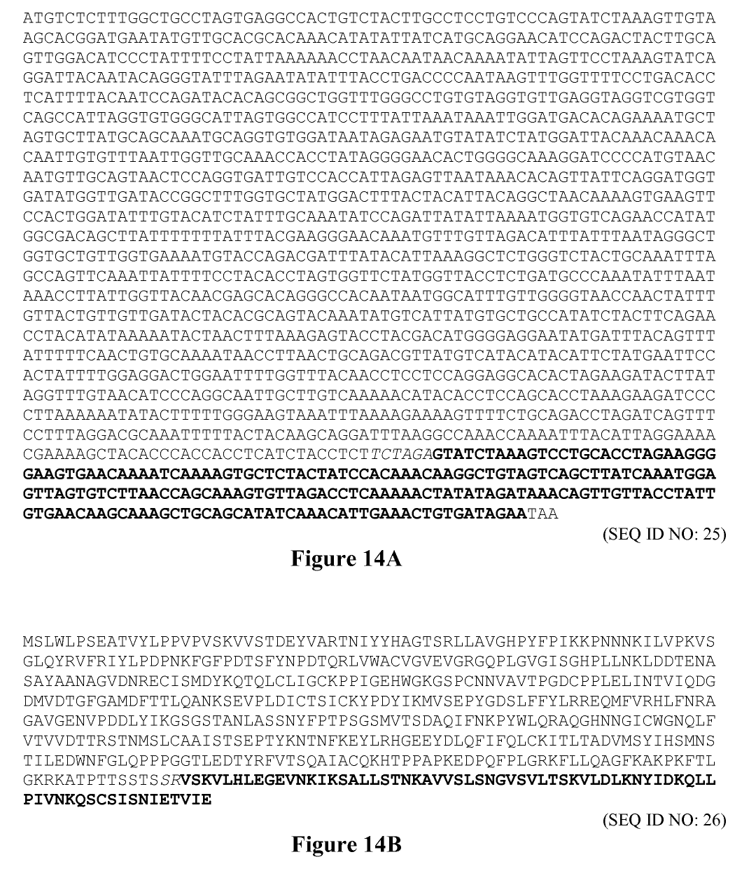

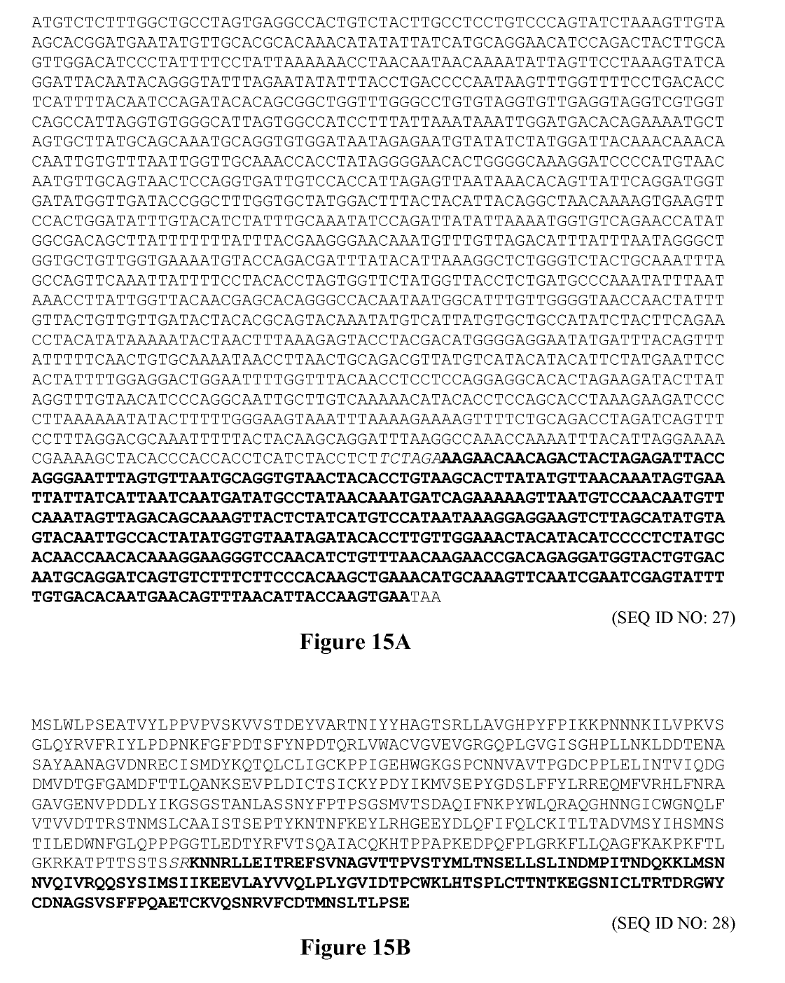

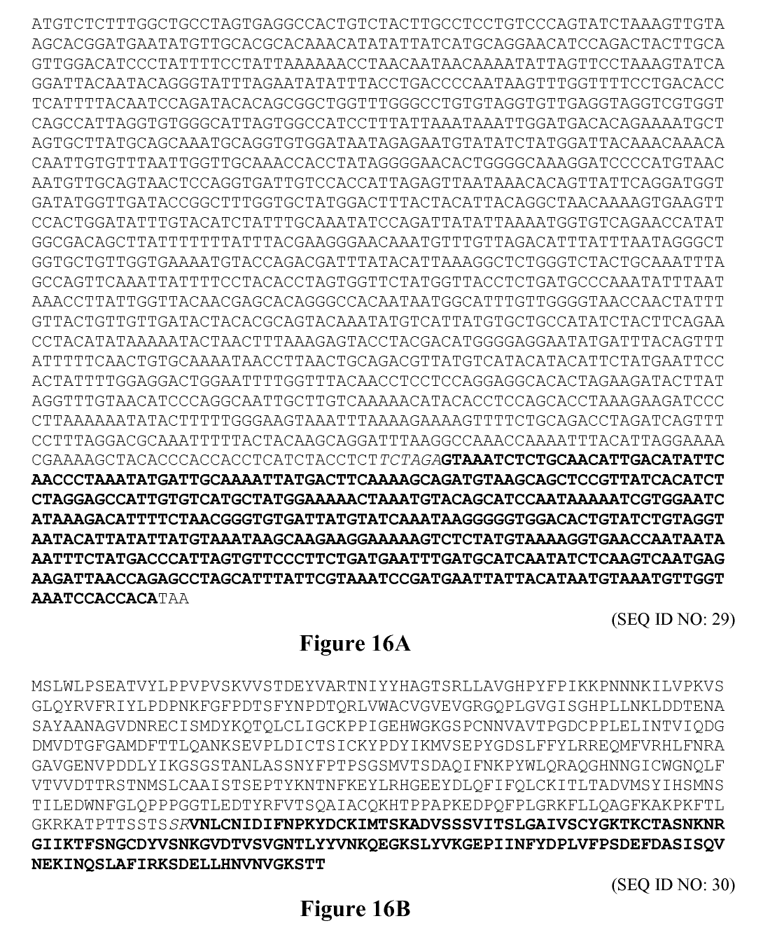

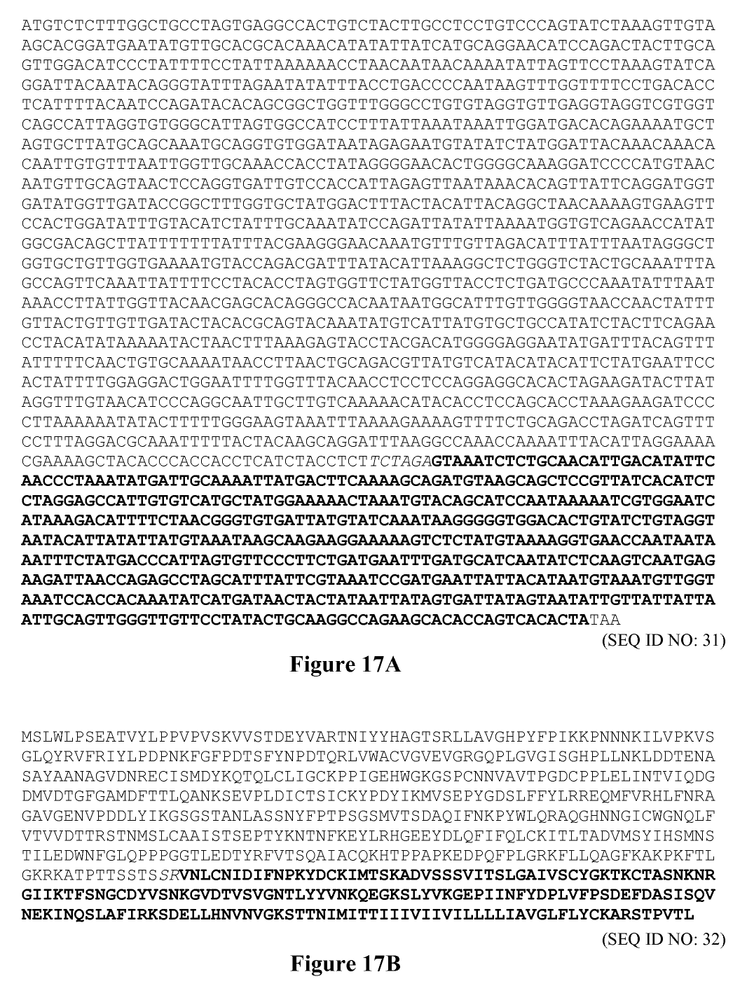

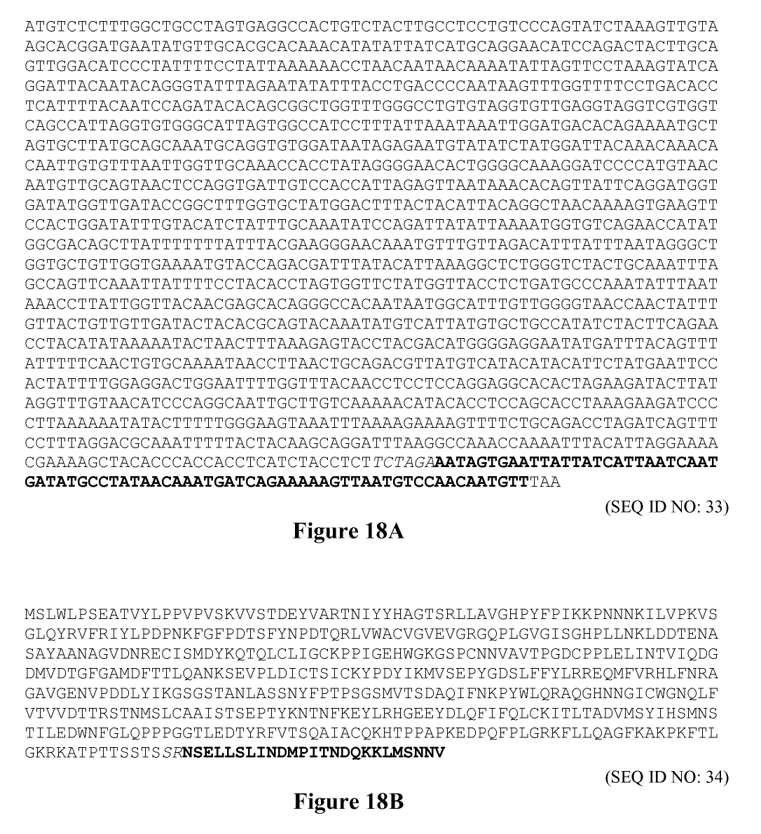

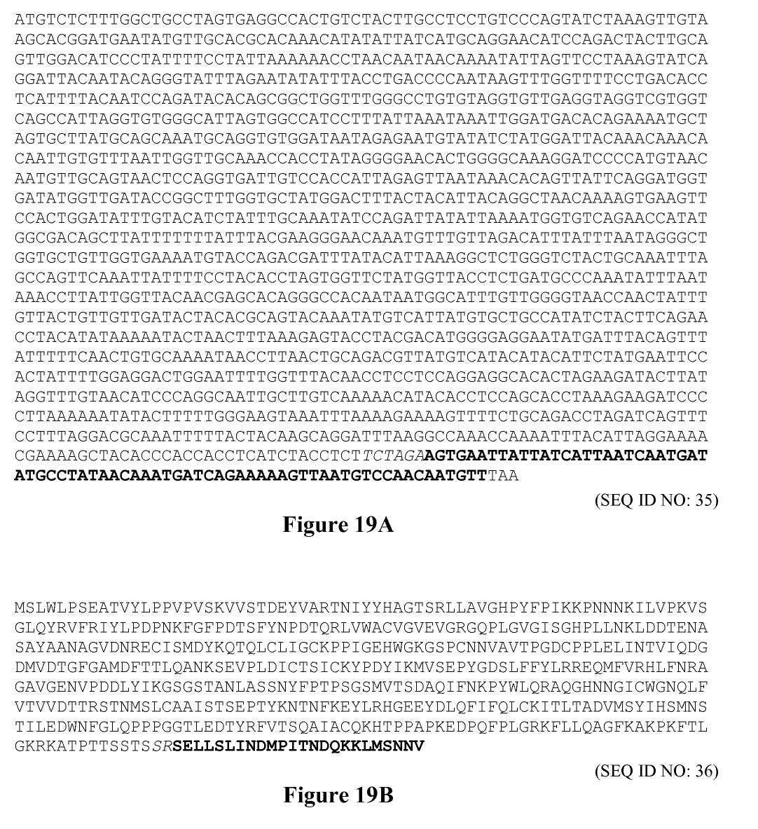

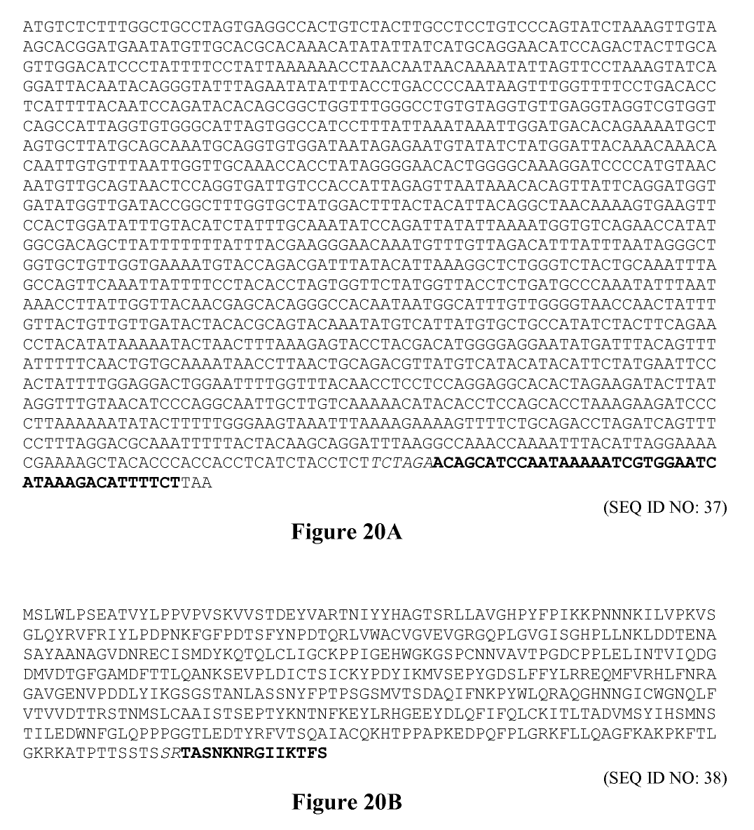

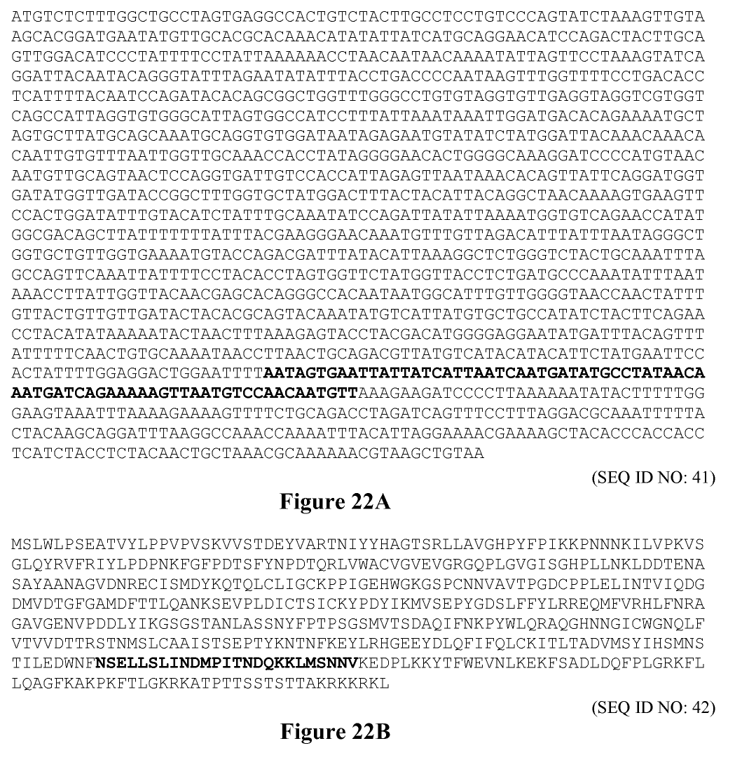

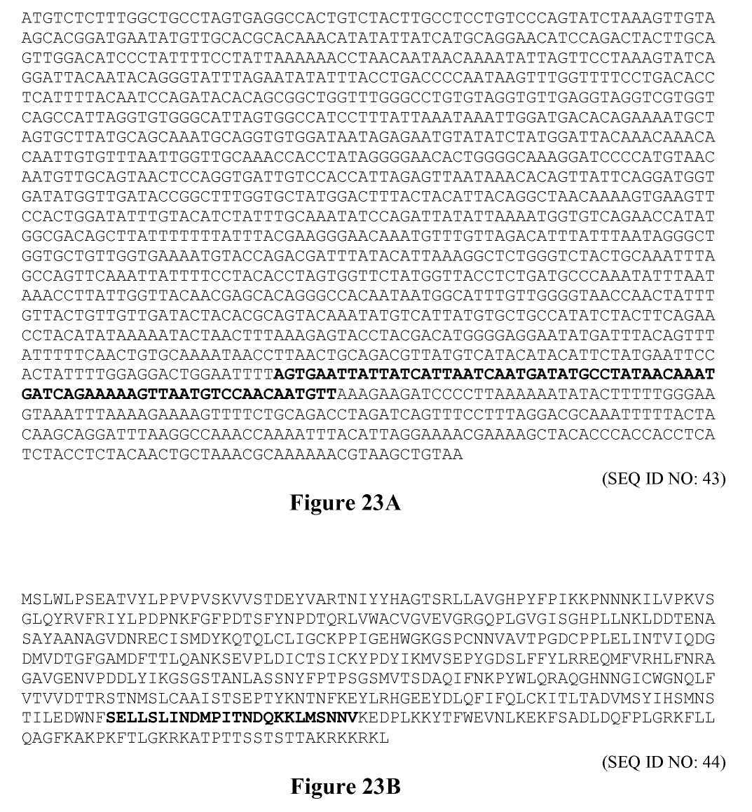

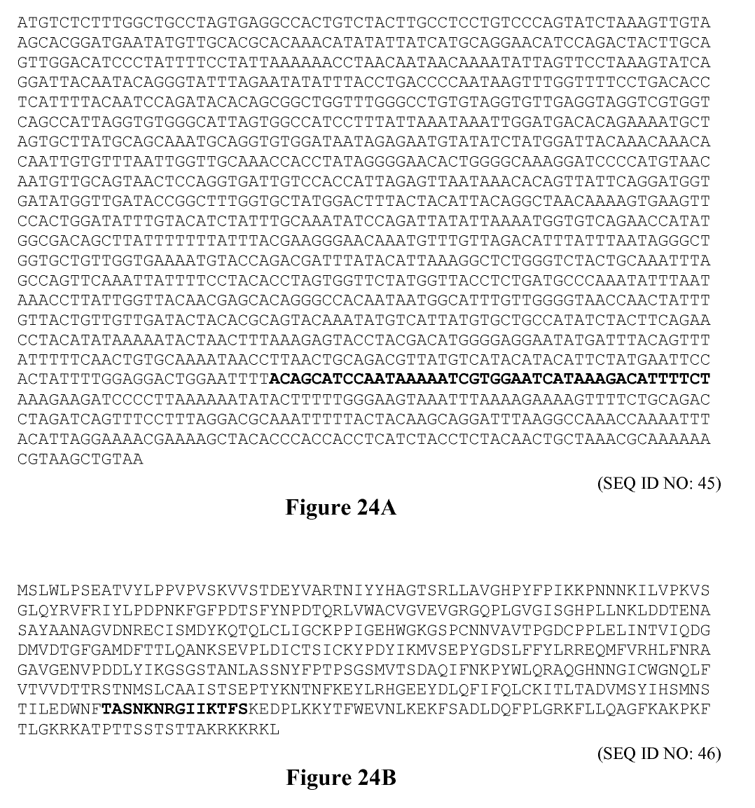

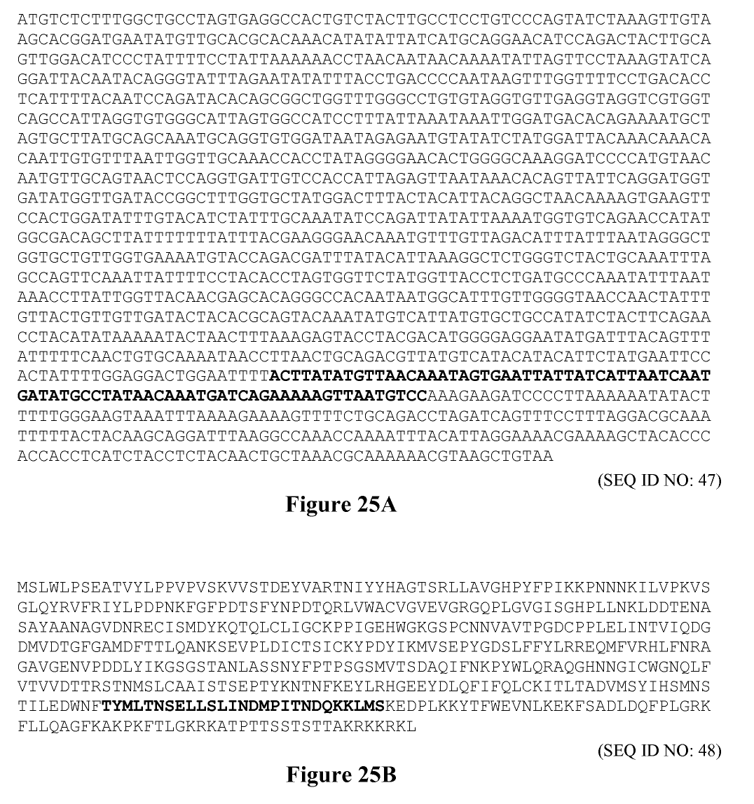

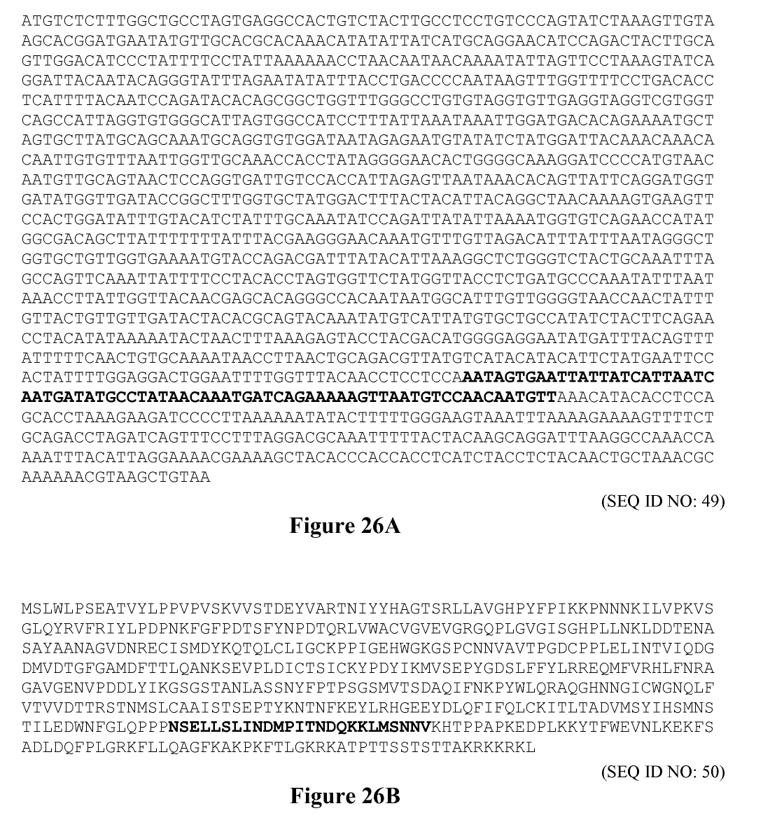

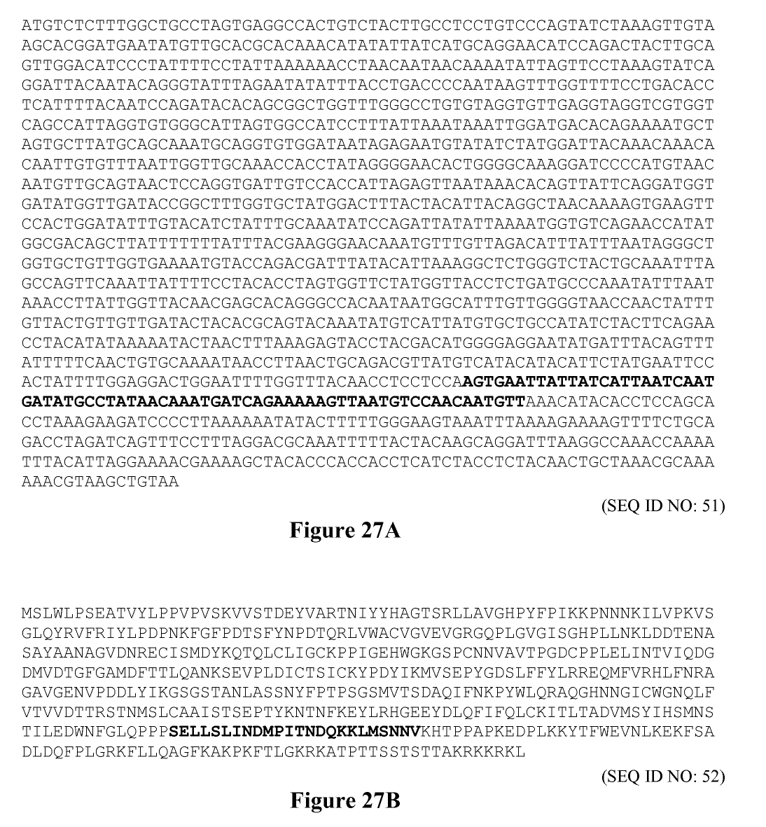

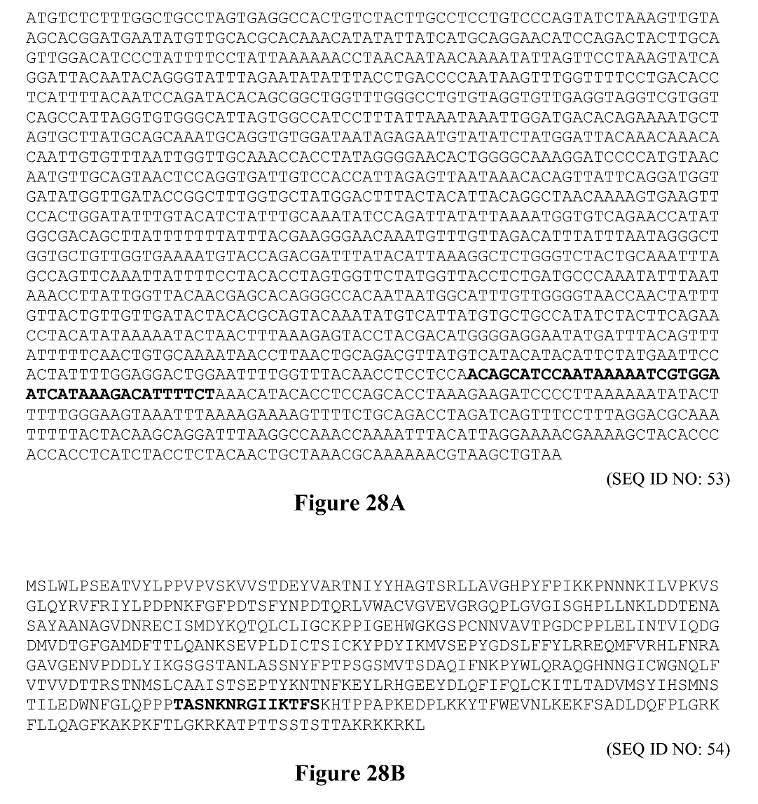

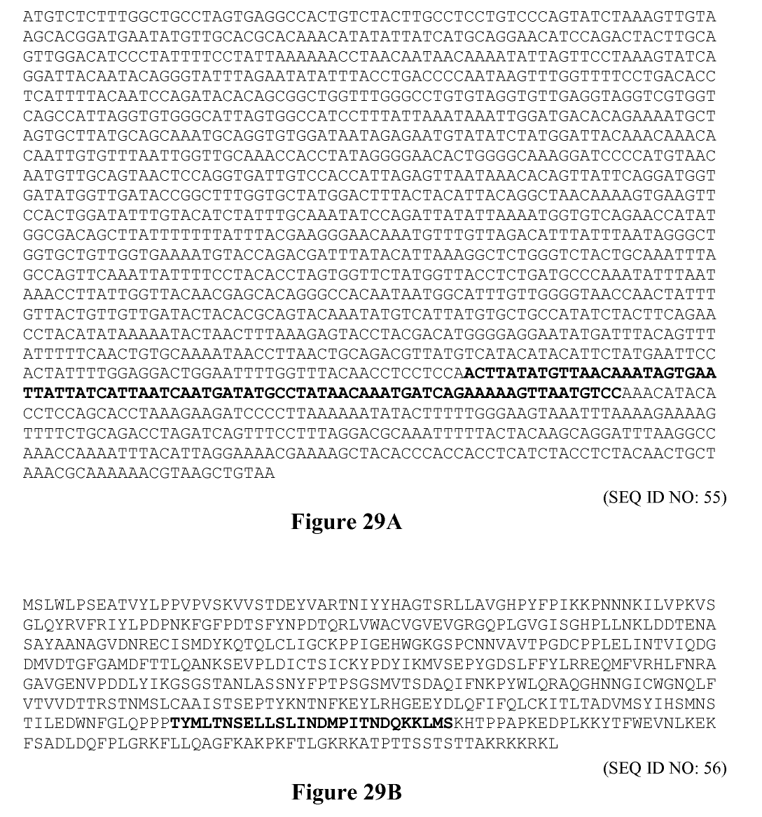

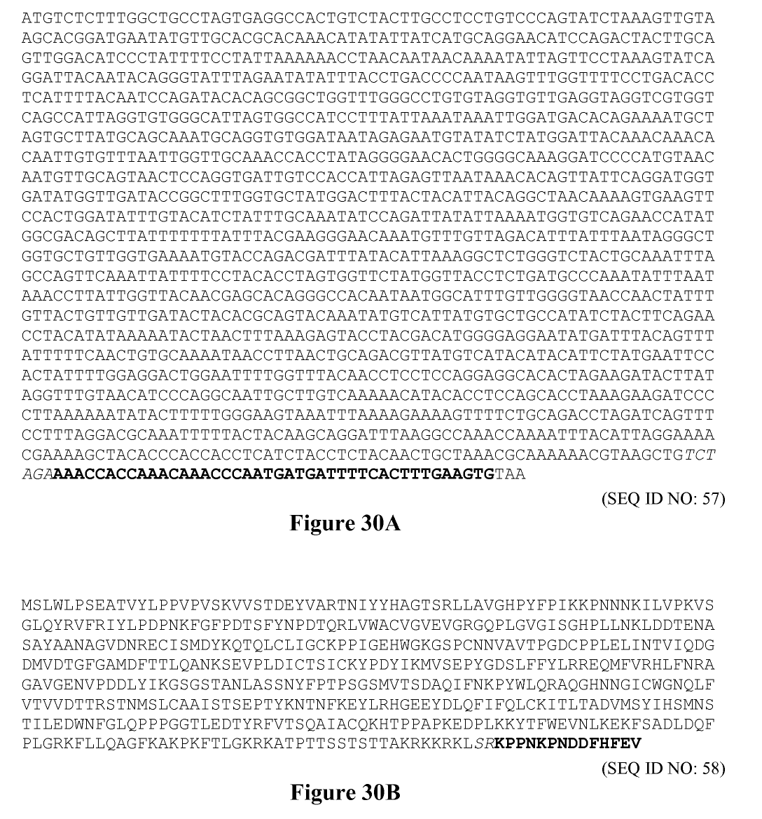

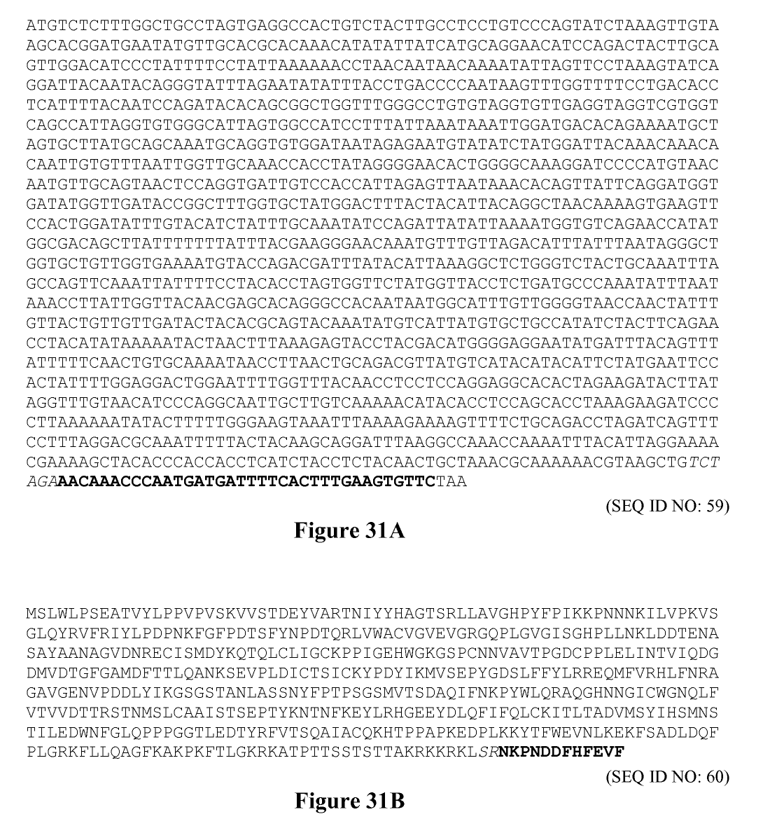

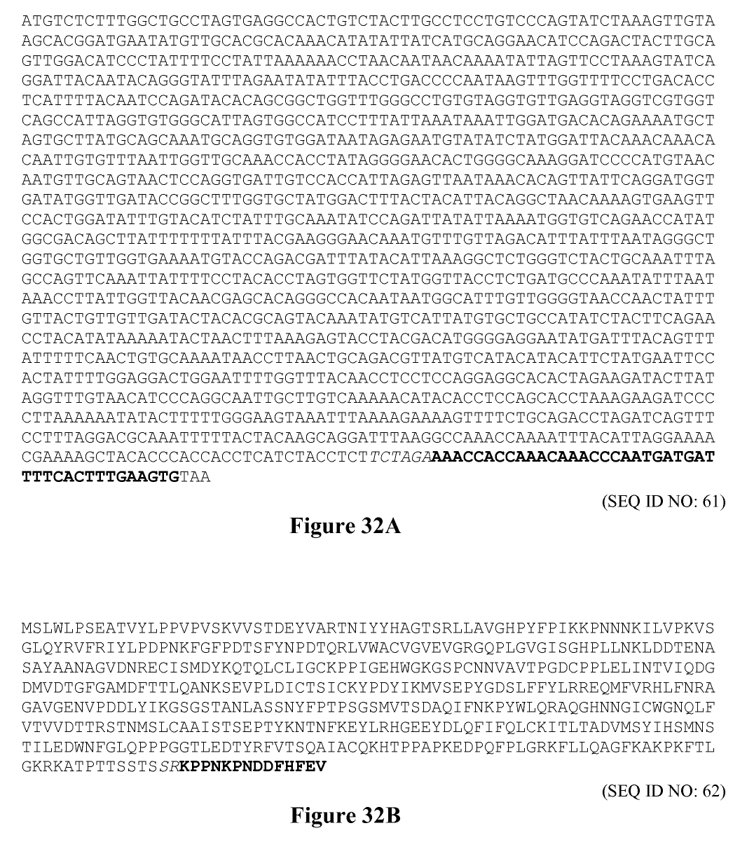

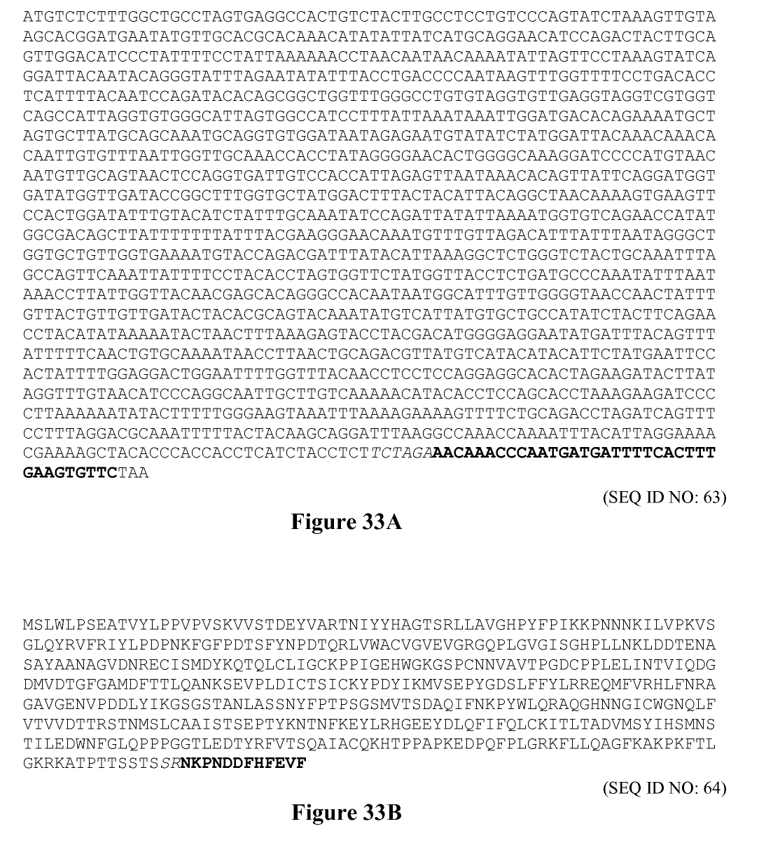

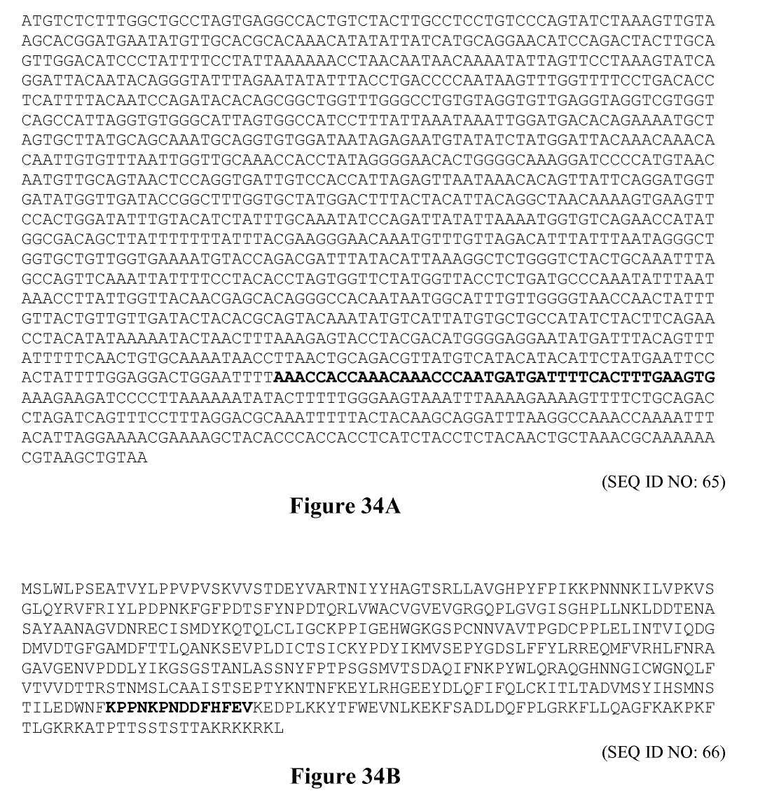

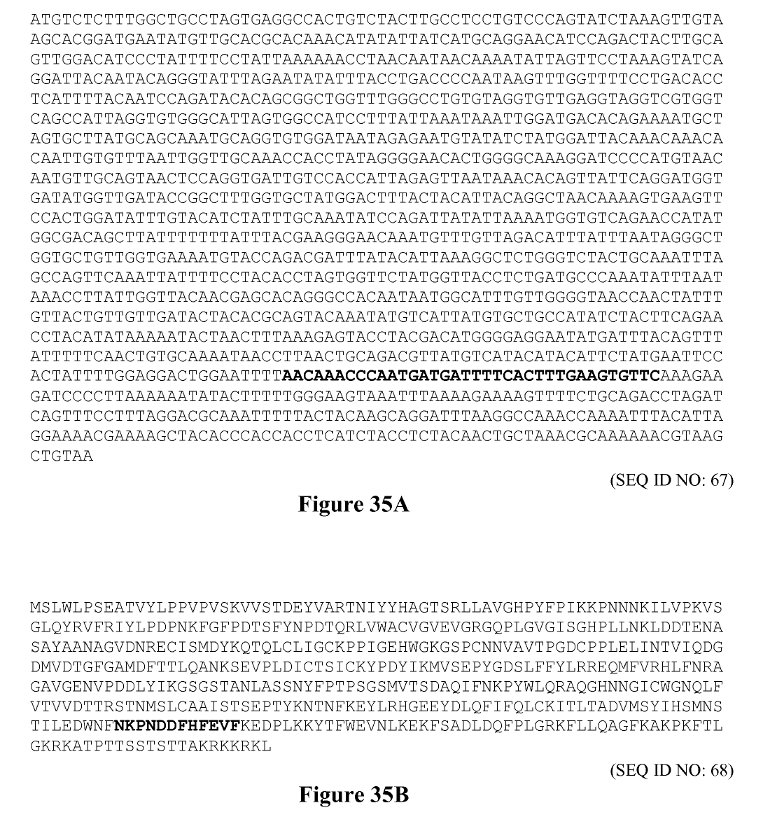

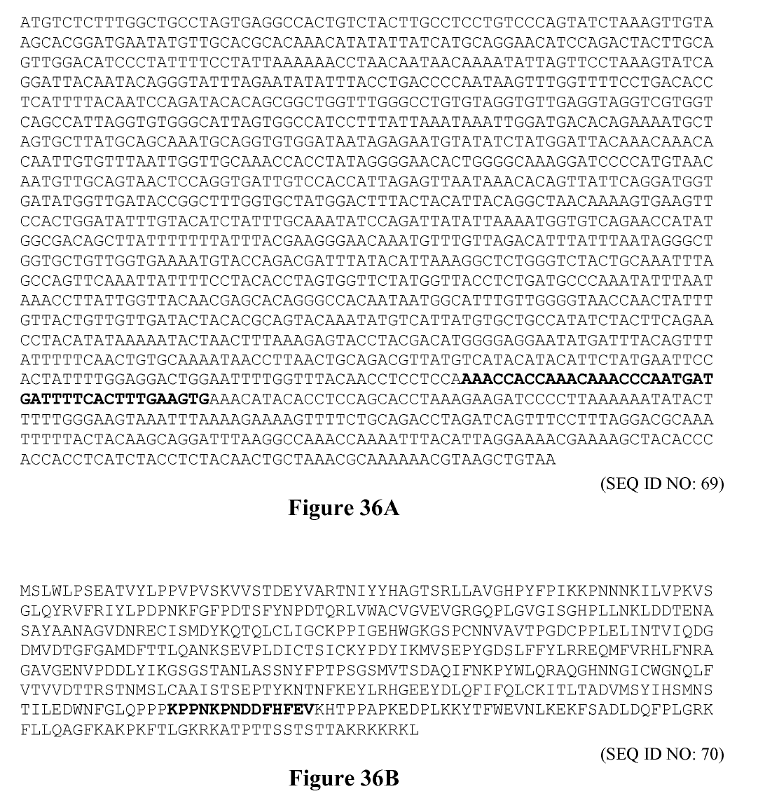

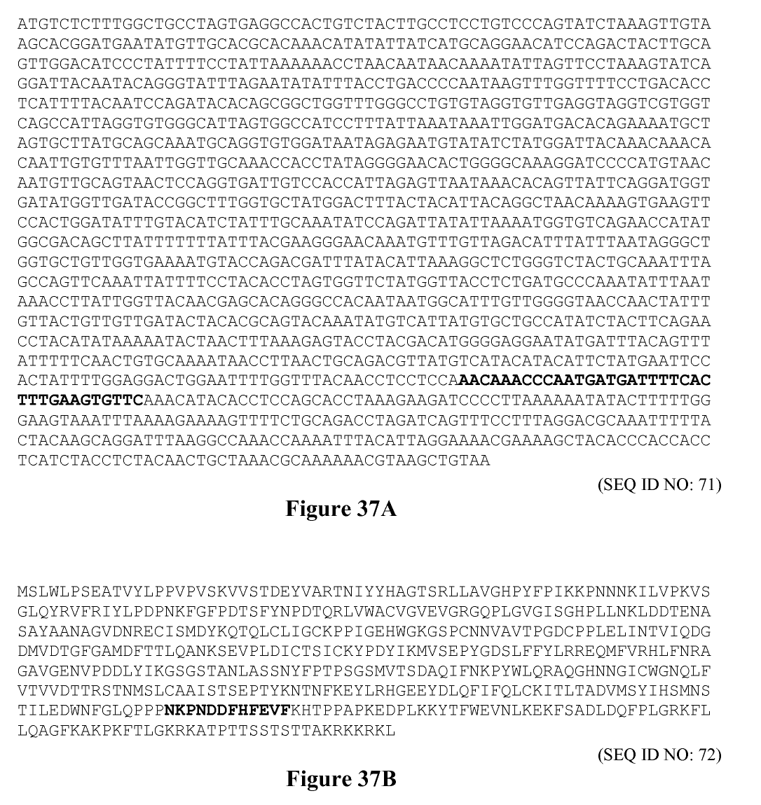

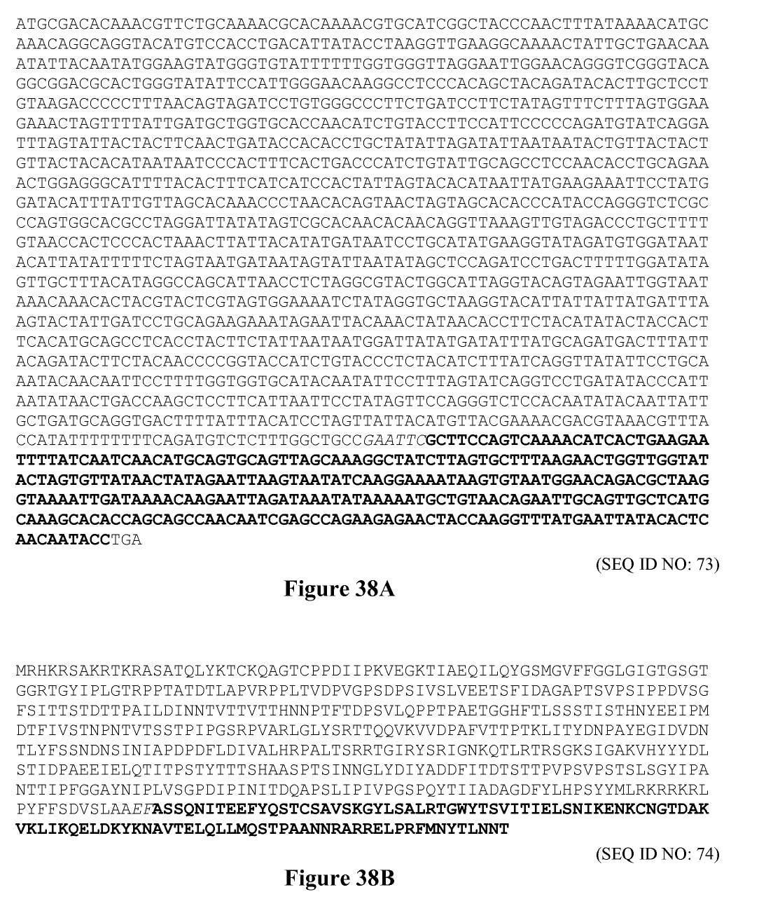

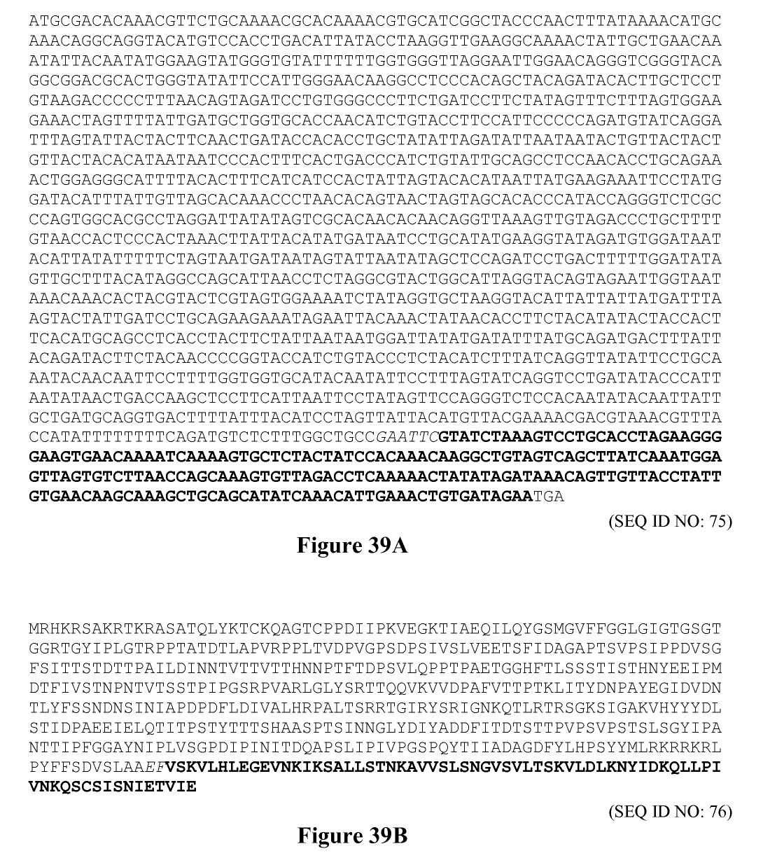

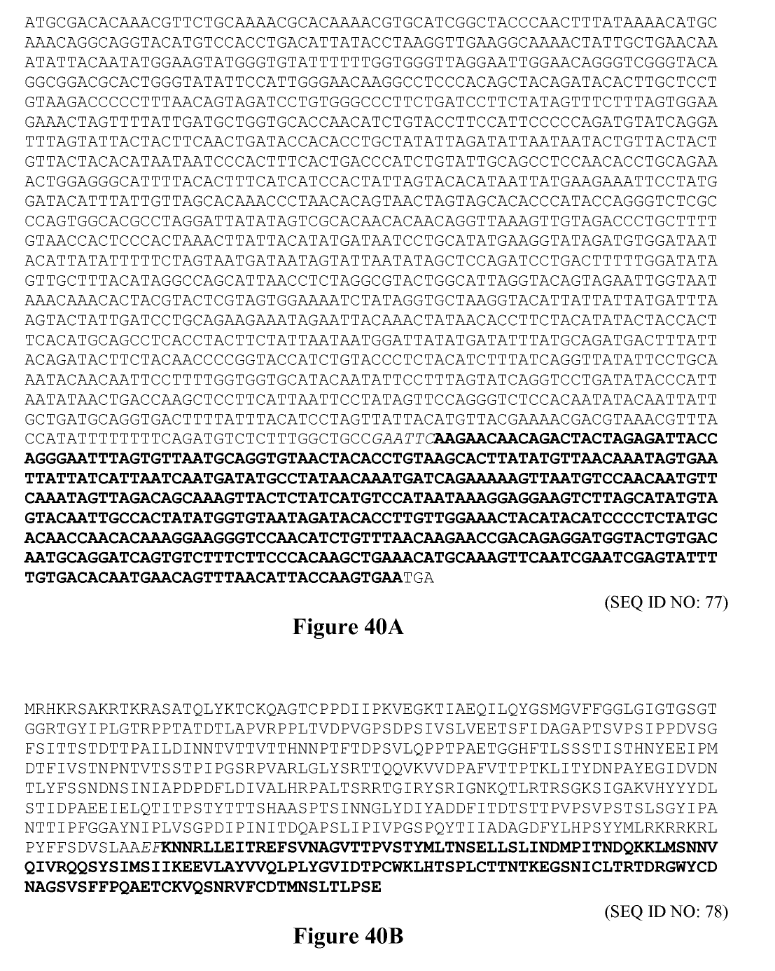

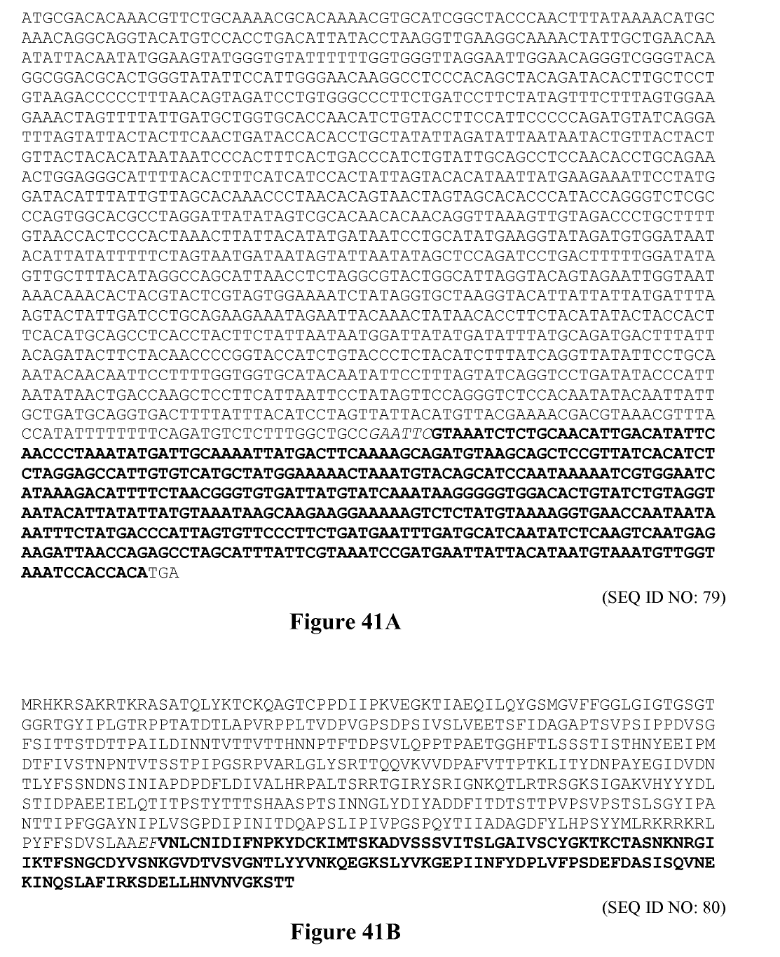

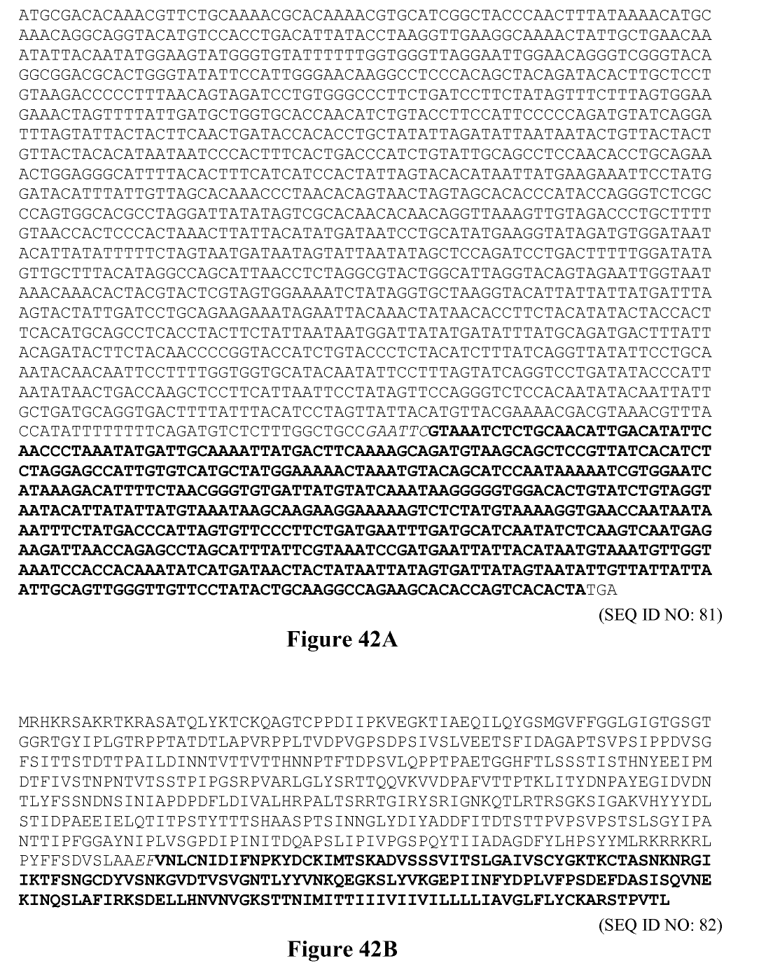

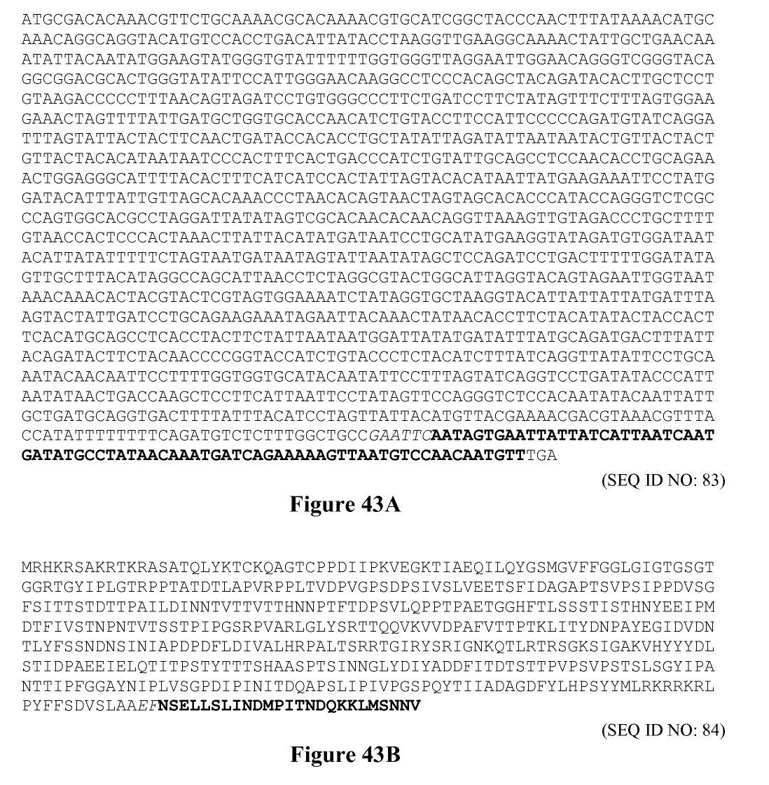

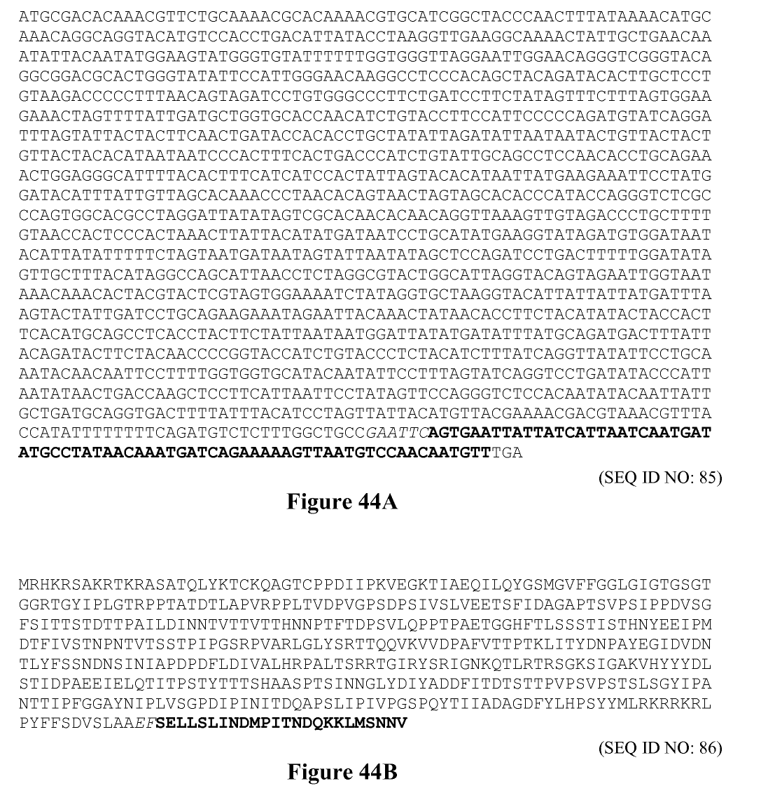

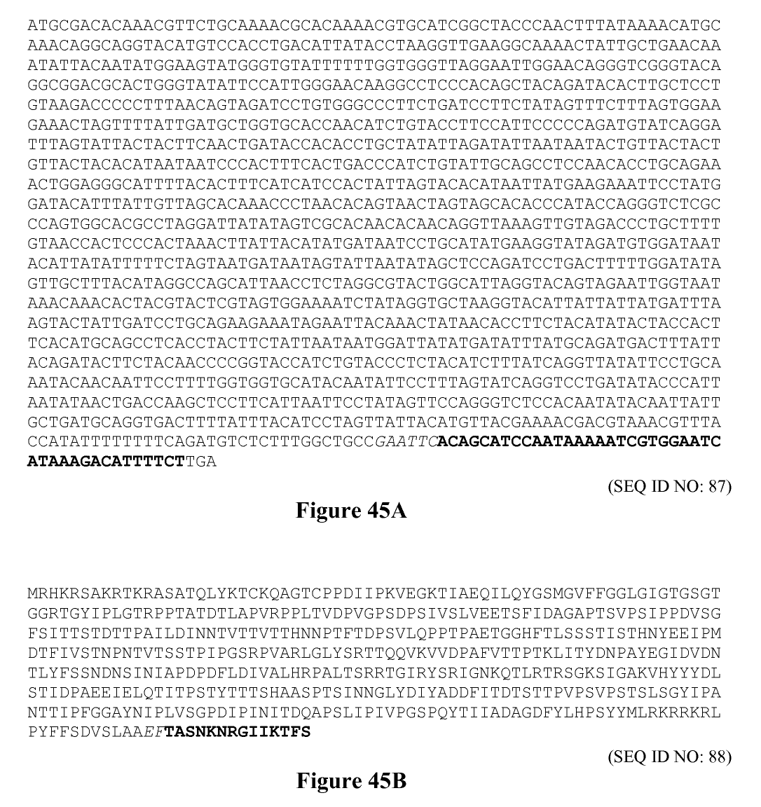

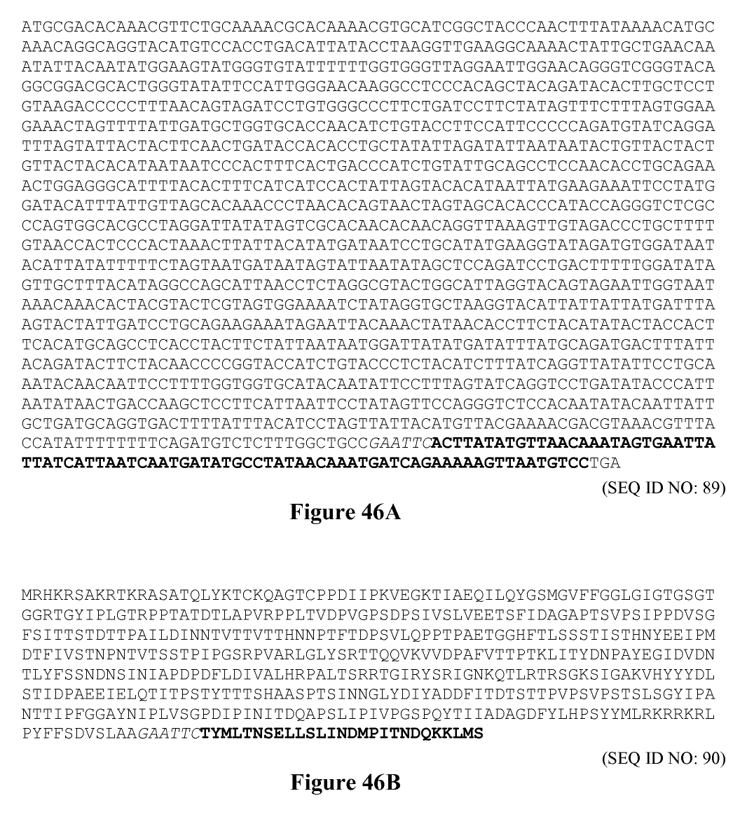

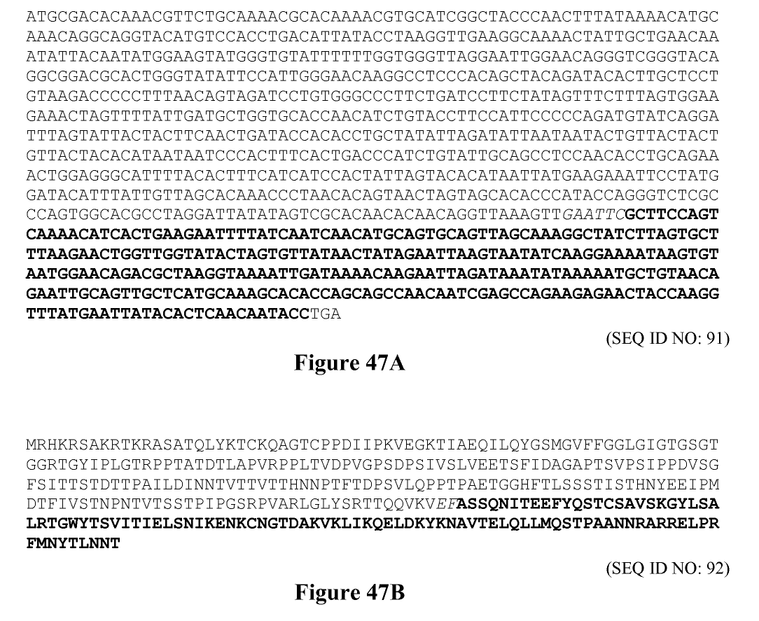

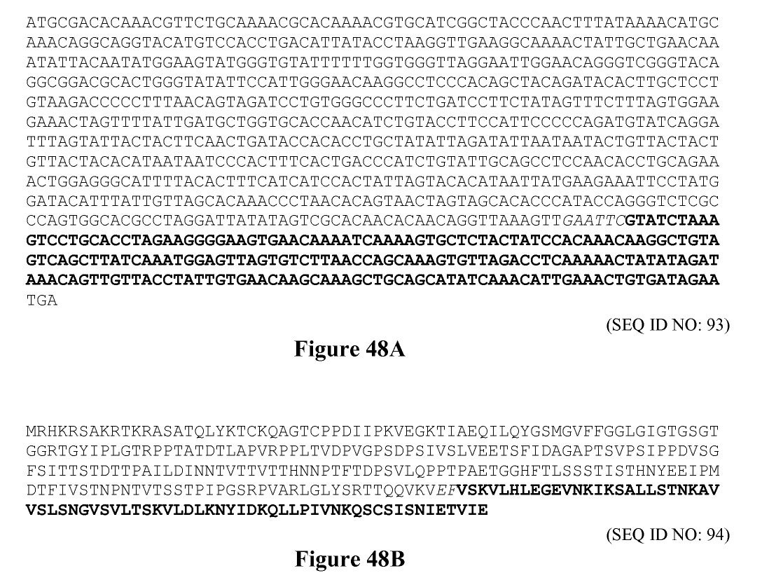

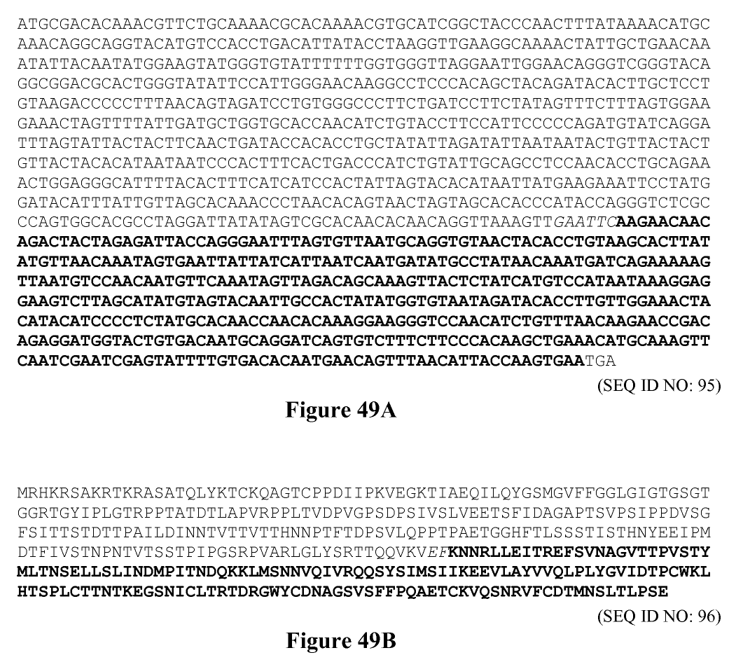

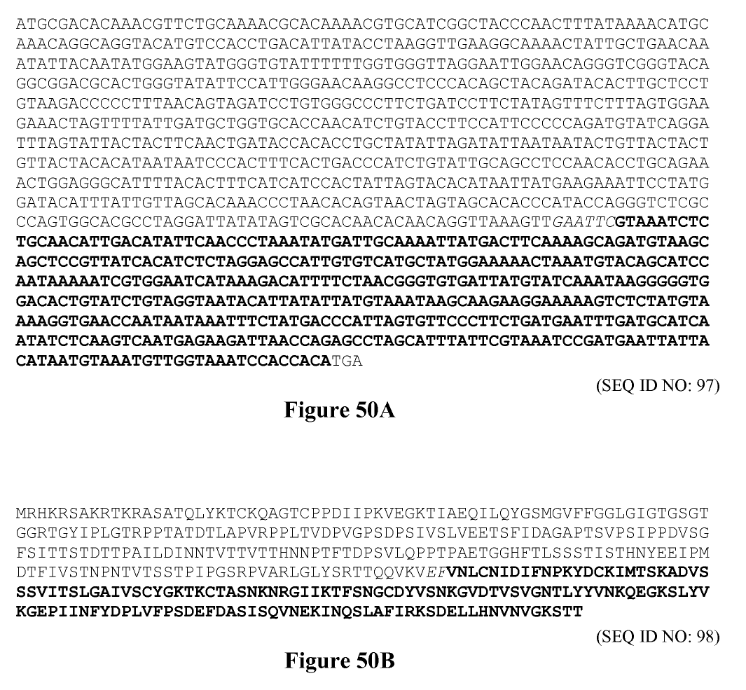

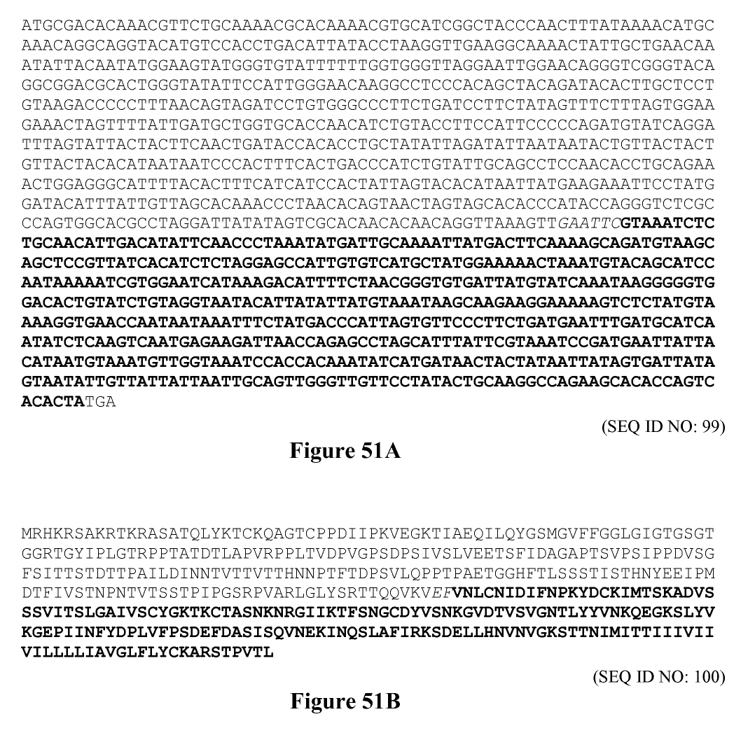

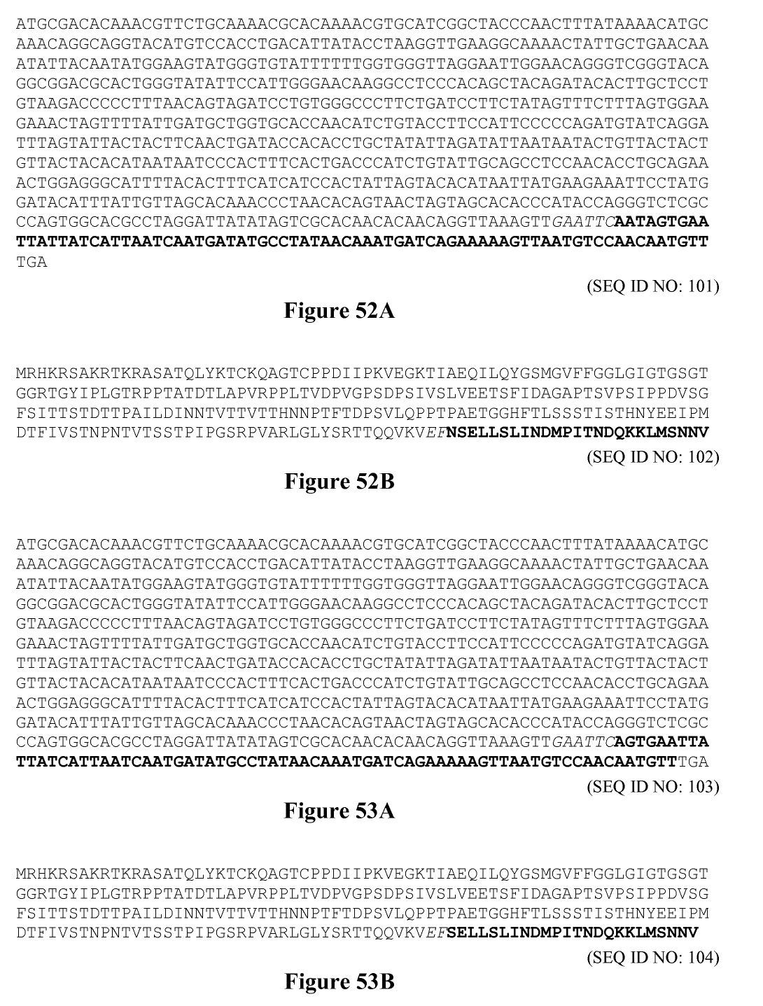

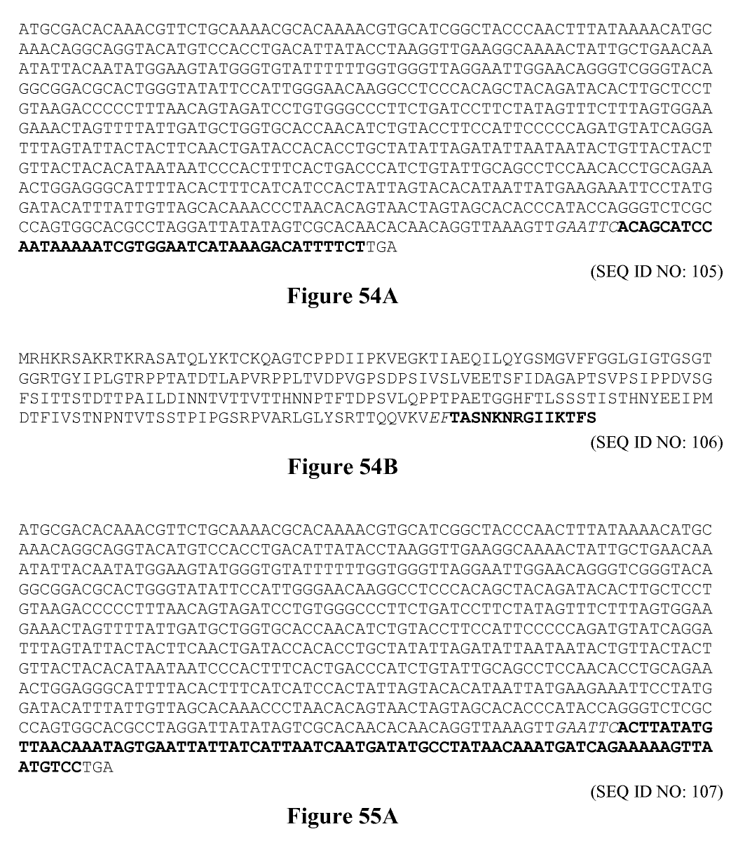

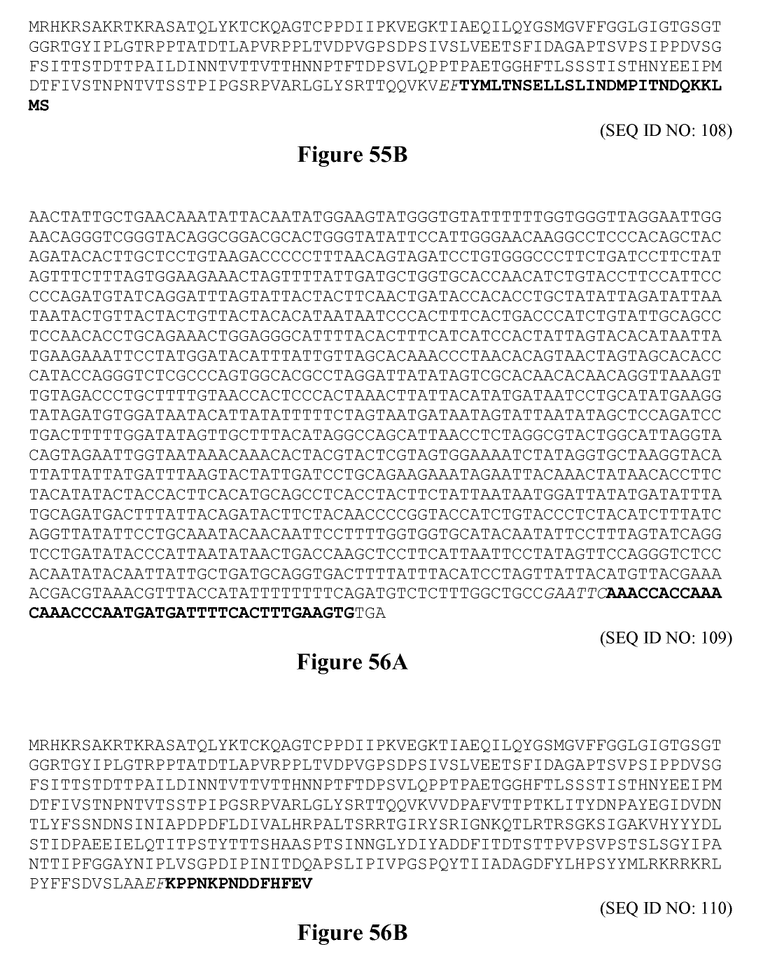

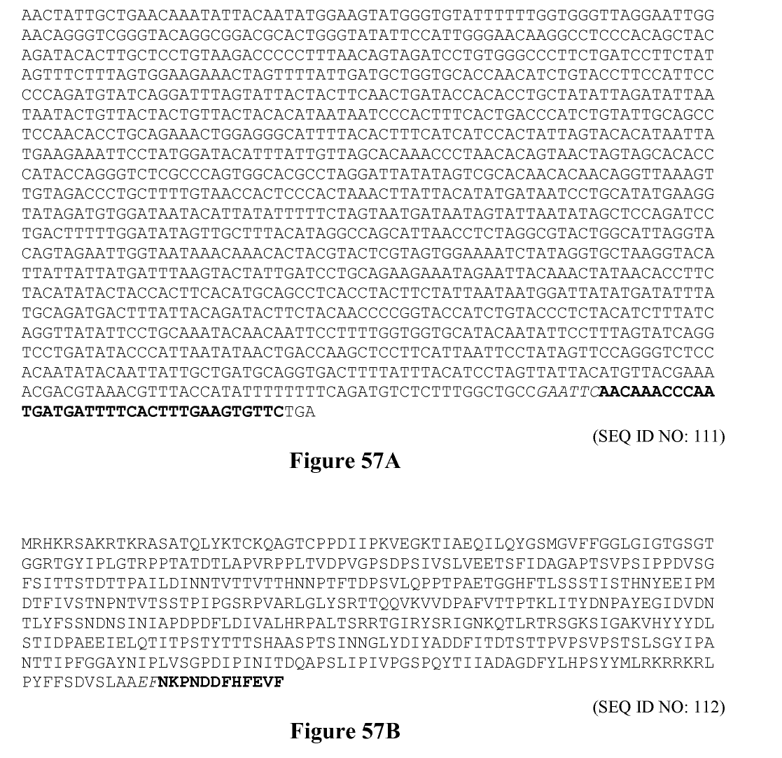

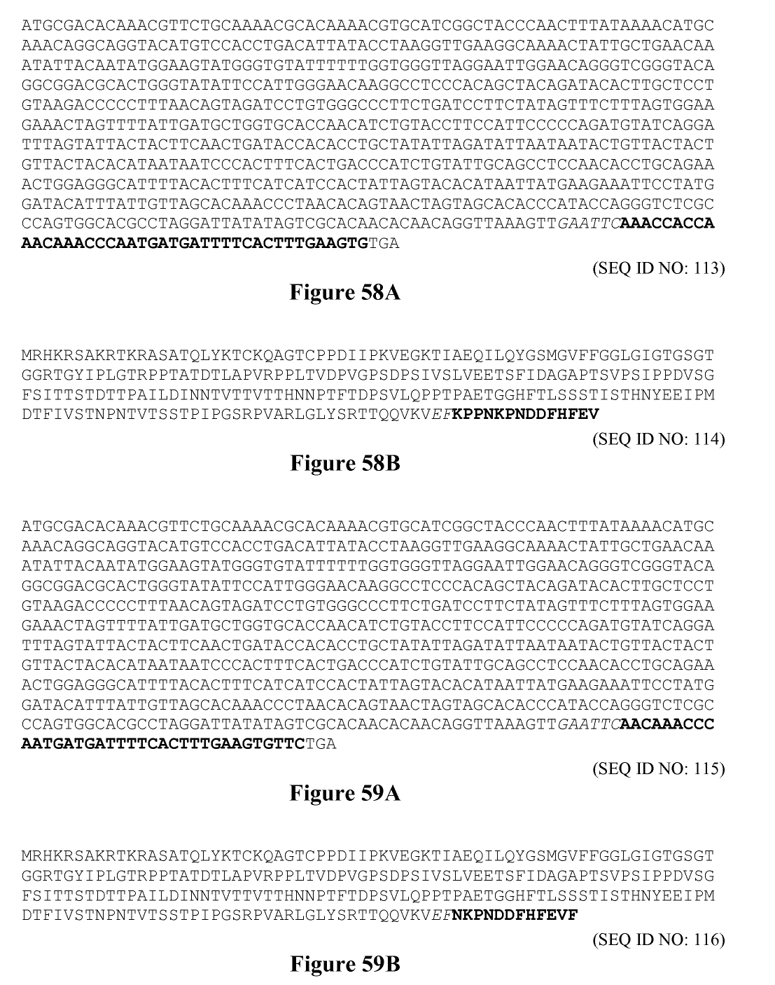

By way of example, and without limitation, suitable L1-RSV fusion proteins include full length L1 polypeptides fused in-frame to one of the above-listed RSV F polypeptides (see SEQ ID NOS: 6, 8, 10, 12, 14, 16, 18, 20, and 22 (FIGS. 4-12)); truncated N-terminal L1 polypeptides fused in-frame to one of the above-listed RSV F polypeptides (see SEQ ID NOS: 24, 26, 28, 30, 32, 34, 36, 38, and 40 (FIGS. 13-21)); truncated C-terminal L1 polypeptides (lacking amino acid residues 2-8, e.g., residues SLWLPSE of HPV-16 L1 as shown in FIGS. 4-12) fused in-frame to one of the above-listed F polypeptides; L1 polypeptides having an h4-domain deletion and one of the above-listed F polypeptides inserted at the h4-deletion site (see SEQ ID NOS: 42, 44, 46, 48, 50, 52, 54, and 56 (FIGS. 22-29)); full length L1 polypeptides fused in-frame to one of the above-listed RSV G polypeptides (see SEQ ID NOS: 58 and 60 (FIGS. 30-31)); truncated N-terminal L1 polypeptides fused in-frame to one of the above-listed RSV G polypeptides (see SEQ ID NOS: 62 and 64 (FIGS. 32-33)); truncated C-terminal L1 polypeptides (lacking amino acid residues 2-8, e.g., residues SLWLPSE of HPV-16 L1 as shown in FIGS. 30-31) fused in-frame to one of the above-listed G polypeptides; L1 polypeptides having an h4-domain deletion and one of the above-listed G polypeptides inserted at the h4-deletion site (see SEQ ID NOS: 66, 68, 70, and 72 (FIGS. 34-37)); full length L2 polypeptides fused in-frame to one of the above-listed RSV F polypeptides (see SEQ ID NOS: 74, 76, 78, 80, 82, 84, 86, 88, and 90 (FIGS. 38-46)); truncated N-terminal L2 polypeptides fused in-frame to one of the above-listed RSV F polypeptides (see SEQ ID NOS: 92, 94, 96, 98, 100, 102, 104, 106, and 108 (FIGS. 47-55)); truncated C-terminal L2 polypeptides fused in-frame to one of the above-listed F polypeptides; full length L2 polypeptides fused in-frame to one of the above-listed RSV G polypeptides (see SEQ ID NOS: 110 and 112 (FIGS. 56-57)); truncated N-terminal L2 polypeptides fused in-frame to one of the above-listed RSV G polypeptides (see SEQ ID NOS: 114 and 116 (FIGS. 58-59)); and truncated C-terminal L2 polypeptides fused in-frame to one of the above-listed G polypeptides.

In addition to these fusion proteins, L1 or L2 polypeptides can be joined in-frame with multiple RSV polypeptides containing different epitopes. For example, the L1 or L2 full-length, N-terminal, or C-terminal polypeptides can be linked in-frame to a first RSV polypeptide containing a first epitope (or more) and a second RSV polypeptide containing a second epitope (or more). Alternatively, both L1-RSV fusion polypeptides and L2-RSV fusion polypeptides can be prepared and expressed for co-assembly, whereby the two fusion proteins contain the same or, more preferably, distinct RSV epitopes. Regardless of the approach for introducing multiple RSV epitopes into the capsomeres or VLPs of the invention, both the first and second epitopes are preferably neutralizing epitopes. In this way, it is possible to use the capsomeres or VLPs to generate a protective immune response that is not dedicated to a single RSV epitope.

The making of VLPs and capsomeres according to this embodiment basically involves the preparation of recombinant genetic constructs using known procedures, followed by the expression of the genetic constructs in recombinant host cells, and then the isolation and purification of the self-assembled VLPs and/or capsomeres.

The genetic constructs encoding the full or partial length L1 polypeptide, full or partial length L2 polypeptide, L1 polypeptide/RSV polypeptide fusion proteins, and L2 polypeptide/RSV polypeptide fusion proteins, can be prepared according to standard recombinant procedures. Basically, DNA molecules encoding the various polypeptide components of the fusion protein (to be prepared) are ligated together to form an in-frame gene fusion that results in, for example, a single open reading frame that expresses a single fusion protein including the papillomavirus capsid polypeptide (L1 or L2) fused to the RSV polypeptide. The DNA coding sequences, or open reading frames, encoding the whole or partial L1 and/or L2 polypeptides and/or fusion proteins can be ligated to appropriate regulatory elements that provide for expression (i.e., transcription and translation) of the fusion protein encoded by the DNA molecule. These regulatory sequences, typically promoters, enhancer elements, transcription terminal signals, etc., are well known in the art.

When a prokaryotic host cell is selected for subsequent transformation, the promoter region used to construct the recombinant DNA molecule (i.e., transgene) should be appropriate for the particular host. As is well known in the art, the DNA sequences of eukaryotic promoters, for expression in eukaryotic host cells, differ from those of prokaryotic promoters. Eukaryotic promoters and accompanying genetic signals may not be recognized in or may not function in a prokaryotic system, and, further, prokaryotic promoters are not recognized and do not function in eukaryotic cells.

Thus, the DNA molecules encoding the polypeptide products to be expressed in accordance with the present invention can be cloned into a suitable expression vector using standard cloning procedures known in the art, including restriction enzyme cleavage and ligation with DNA ligase as described by Sambrook et al., Molecular Cloning: A Laboratory Manual, Second Edition, Cold Spring Harbor Press, NY (2001), and Ausubel et al., Current Protocols in Molecular Biology, John Wiley & Sons, New York, N.Y. (2008), each of which is hereby incorporated by reference in its entirety. Recombinant molecules, including plasmids, can be introduced into cells via transformation, particularly transduction, conjugation, mobilization, or electroporation. Once these recombinant plasmids are introduced into unicellular cultures, including prokaryotic organisms and eukaryotic cells, the cells are grown in tissue culture and vectors can be replicated.

For the recombinant expression of papillomavirus L1 and/or L2 fusion proteins, and resulting capsomere and/or VLP assembly, the recombinant vectors produced above are used to infect a host cell. Any number of vector-host combinations can be employed, including plant cell vectors (Agrobacterium) and plant cells, yeast vectors and yeast hosts, baculovirus vectors and insect host cells, vaccinia virus vectors and mammalian host cells, or plasmid vectors in E. coli. Additional mammalian expression vectors include those derived from adenovirus adeno-associated virus, nodavirus, and retroviruses.

The capsomeres and/or VLPs of the present invention are preferably formed in Sf-9 insect cells upon expression of the L1 and optionally L2 proteins or polypeptides using recombinant baculovirus. General methods for handling and preparing baculovirus vectors and baculovirus DNA, as well as insect cell culture procedures, are outlined in The Molecular Biology of Baculoviruses, Doerffer et al., Eds. Springer-Verlag, Berlin, pages 31-49; Kool et al., “The Structural and Functional Organization of the Autographa californica Nuclear Polyhedrosis Virus Genome,” Arch. Virol. 130:1-16 (1993); Kirnbauer et al., “Efficient Self-assembly of Human Papillomavirus Type 16 L1 and L1-L2 into Virus-like Particles,” J. Virol. 67(12): 6929-6936 (1993); Volpers et al., “Binding and Internalization of Human Papillomavirus Type 33 Virus-like Particles by Eukaryotic Cells,” J. Virol. 69:3258-3264 (1995); Rose et al., “Expression of Human Papillomavirus Type 11 L1 Protein in Insect Cells: in vivo and in vitro Assembly of Viruslike Particles,” J. Virol. 67(4): 1936-1944 (1993), each of which is hereby incorporated by reference in its entirety).

However, recombinant expression vectors and regulatory sequences suitable for expression of papillomavirus polypeptides in yeast or mammalian cells are well known and can be used in the present invention (see Hagensee et al., “Self-assembly of Human Papillomavirus Type 1 Capsids by Expression of the L1 Protein Alone or by Coexpression of the L1 and L2 Capsid Proteins,” J. Virol. 67(1):315-22 (1993); Sasagawa et al., “Synthesis and Assembly of Virus-like Particles of Human Papillomaviruses Type 6 and Type 16 in Fission Yeast Schizosaccharomyces pombe,” Virology 2016:126-195 (1995); Buonamassa et al., “Yeast Coexpression of Human Papillomavirus Types 6 and 16 Capsid Proteins,” Virol. 293(2):335-344 (2002); U.S. Pat. No. 7,112,330 to Buonamassa et al.; U.S. Patent Publ. No. 20080166371 to Jansen et al., each of which is hereby incorporated by reference in its entirety).

Regardless of the host-vector system utilized for the recombinant expression and self-assembly of capsomeres and/or VLPs, these products can be isolated from the host cells, and then purified using known techniques. For example, the purification of the VLPs or capsomeres can be achieved very simply by means of centrifugation in CsCl or sucrose gradients (Kirnbauer et al., “Efficient Self-assembly of Human Papillomavirus Type 16 L1 and L1-L2 into Virus-like Particles,” J. Virol. 67(12): 6929-6936 (1993); Sasagawa et al., “Synthesis and Assembly of Virus-like Particles of Human Papillomaviruses Type 6 and Type 16 in Fission Yeast Schizosaccharomyces pombe,” Virology 2016:126-195 (1995); Volpers et al., “Binding and Internalization of Human Papillomavirus Type 33 Virus-like Particles by Eukaryotic Cells,” J. Virol. 69:3258-3264 (1995); Rose et al., “Expression of Human Papillomavirus Type 11 L1 Protein in Insect Cells: in vivo and in vitro Assembly of Viruslike Particles,” J. Virol. 67(4):1936-1944 (1993); Rose et al., “Serologic Differentiation of Human Papillomavirus (HPV) Types 11, 16, and 18 L1 Virus-like Particles (VLPs),” J. Gen. Virol., 75:2445-2449 (1994), each of which is hereby incorporated by reference in its entirety). Substantially pure VLP or capsomere preparations can be used as the active agent in a vaccine, as discussed hereinafter.

Alternatively, for expression in prokaryotes such as E. coli, a GST-fusion protein or other suitable chimeric protein can be expressed recombinantly, and thereafter purified and the GST portion cleaved to afford a self-assembly competent L1-RSV polypeptide that forms capsomeres or VLPs. See Chen et al., “Papillomavirus Capsid Protein Expression in Escherichia coli: Purification and Assembly of HPV11 and HPV16 L1,” J Mol Biol. 307:173-182 (2001), which is hereby incorporated by reference in its entirety. The resulting VLPs or capsomeres can be purified again to separate the structural assemblies from host cell by-products.

According to another embodiment of the present invention, non-chimeric, recombinant VLPs or capsomeres are first produced and purified, and then are thereafter modified by chemically conjugating the RSV polypeptide to the VLP or capsomere surface via small cross-linking molecules (Ionescu et al., “Pharmaceutical and Immunological Evaluation of Human Papillomavirus Virus Like Particle as an Antigen Carrier,” J Pharm Sci 95:70-79 (2006), which is hereby incorporated by reference in its entirety). As a result of this conjugation, the resulting VLP or capsomere product is effectively decorated with anywhere from several hundred up to several thousand of the conjugated molecules per VLP (or corresponding amount per capsomere). This level of conjugation is capable of eliciting a strong, protective antibody response against the conjugated peptide sequence (Ionescu et al., “Pharmaceutical and Immunological Evaluation of Human Papillomavirus Virus Like Particle as an Antigen Carrier,” J Pharm Sci 95:70-79 (2006), which is hereby incorporated by reference in its entirety).

The RSV polypeptides can be conjugated with any suitable linker molecule, but preferably a hetero-bifunctional cross linker molecule. A number of hetero-bifunctional cross-linker molecules are known in the art, and are commercially available. Exemplary hetero-bifunctional crosslinker molecules include, without limitation, N-succinimidyl 3-(2-pyridyldithio)-propionate (“SPDP”), succinimidyl 6-(3-[2-pyridyldithio]-propionamido)hexanoate (“LC-SPDP”), sulfosuccinimidyl 4-[N-maleimidomethyl]cyclohexane-1-carboxylate (“Sulfo-SMCC”), succinimidyl-4-(N-maleimidomethyl)cyclohexane-1-carboxylate (“SMCC”), succinimidyl-4-[N-maleimidomethyl]cyclohexane-1-carboxy-[6-amidocaproate], [N-e-maleimidocaproyloxy]succinimide ester (“EMCS”), [N-e-maleimidocaproyloxy]sulfosuccinimide ester (“Sulfo-EMCS”), N-[g-maleimidobutyryloxy]succinimide ester (“GMBS”), N-[g-maleimidobutyryloxy]sulfosuccinimide ester (“Sulfo-GMBS”), N-[k-maleimidoundecanoyloxy]sulfosuccinimide ester (“Sulfo-KMUS”), 4-succinimidyloxycarbonyl-methyl-a-[2-pyridyldithio]toluene (“SMPT”), 4-sulfosuccinimidyl-6-methyl-a-(2-pyridyldithio)toluamido]hexanoate (“Sulfo-LC-SMPT”), m-maleimidobenzoyl-N-hydroxysuccinimide ester (“MBS”), m-maleimidobenzoyl-N-hydroxysulfosuccinimide ester (“Sulfo-MBS”), N-succinimidyl[4-iodoacetyl]aminobenzoate (“SIAB”), N-sulfosuccinimidyl[4-iodoacetyl]aminobenzoate (“Sulfo-SIAB”), succinimidyl 4-[p-maleimidophenyl]butyrate (“SMPB”), sulfosuccinimidyl 4-[p-maleimidophenyl]butyrate (“Sulfo-SMPB”), N-(a-maleimidoacetoxy) succinimide ester (“AMAS”), N-[4-(p-azidosalicylamido) butyl]-3″-(2″-pyridyldithio)propionamide (“APDP”), N-[β-maleimidopropyloxy]succinimide ester (“BMPS”), N-e-maleimidocaproic acid (“EMCA”), N-succinimidyl iodoacetate (“SIA”), and succinimidyl-6-[β-maleimidopropionamido]hexanoate (“SMPH”).

According to one approach, illustrated in the accompanying examples, a bi-functional linker molecule such as succinimidyl-6-[β-maleimidopropionamido]hexanoate (“SMPH”) can be reacted in excess with VLPs or capsomeres. SMPH is an amine- and sulfhydryl-reactive hetero-bifunctional cross-linker. The SMPH-bound VLPs or capsomeres can be exposed to a suitable RSV polypeptide (containing a desired epitope and, preferably an N-terminal or C-terminal cysteine residue) under conditions effective to allow for covalent binding of the RSV polypeptide to the linker molecule. One approach for carrying out this embodiment of the present invention is illustrated in Example 5. After conjugation, the chimeric VLPs or capsomeres can be purified (to remove) unreacted peptide via dialysis.

Having purified the capsomeres or VLPs, these materials can be introduced into pharmaceutical compositions that are suitable for use in immunizing an individual against RSV infection. Preferably, the capsomeres or VLPs are present in the pharmaceutical compositions in an amount that is effective to induce a high-titer neutralizing antibody response against the RSV epitopes and/or a TH-1 dominant CTL response. Thus, effective amounts include an amount ranging from about 1 to about 500 μg of the VLPs or capsomeres, preferably about 5 to about 200 μg, more preferably about 10 to about 100 μg, most preferably 20 to about 80 μg.

The pharmaceutical compositions of the present invention preferably include a pharmaceutically acceptable carrier. Acceptable pharmaceutical carriers include solutions, suspensions, emulsions, excipients, powders, or stabilizers. The carrier should be suitable for the desired mode of delivery, discussed infra.

For example, compositions suitable for injectable use (e.g., intravenous, intra-arterial, intramuscular, etc.) may include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. Suitable adjuvants, carriers and/or excipients, include, but are not limited to sterile liquids, such as water and oils, with or without the addition of a surfactant and other pharmaceutically and physiologically acceptable carrier. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions. The ability of papillomavirus VLP formulations to induce an effective immune response following intramuscular injection has been well established by current Gardasil® vaccine.

Oral dosage formulations can include standard carriers such as pharmaceutical grades of mannitol, lactose, starch, magnesium stearate, sodium saccharine, cellulose, magnesium carbonate, etc. Suitable carriers include lubricants and inert fillers such as lactose, sucrose, or cornstarch. In another embodiment, these compounds are tableted with conventional tablet bases such as lactose, sucrose, or cornstarch in combination with binders like acacia, gum gragacanth, cornstarch, or gelatin; disintegrating agents such as cornstarch, potato starch, or alginic acid; a lubricant like stearic acid or magnesium stearate; and sweetening agents such as sucrose, lactose, or saccharine; and flavoring agents such as peppermint oil, oil of wintergreen, or artificial flavorings. Generally, the ingredients are supplied either separately or mixed together in unit dosage form, for example, as a dry lyophilized powder or water free concentrate in a hermetically sealed container such as an ampule or sachette indicating the quantity of active agent. The ability of papillomavirus VLP formulations to induce an effective immune response following oral delivery is described in Gerber et al., “Human Papillomavirus Virus-Like Particles Are Efficient Oral Immunogens when Coadministered with Escherichia coli Heat-Labile Enterotoxin Mutant R192G or CpG DNA,” J. Virol. 75(10):4752-4760 (2001); Sasagawa et al., “A Human Papillomavirus Type 16 Vaccine by Oral Delivery of L1 Protein,” Virus Res. 110(1-2):81-90 (2005), each of which is hereby incorporated by reference in its entirety.

Formulations suitable for transdermal delivery can also be prepared in accordance with the teachings of U.S. Pat. No. 7,247,433 to Rose, which is hereby incorporated by reference in its entirety.

Formulations suitable for intranasal nebulization or bronchial aerosolization delivery are also known and can be used in the present invention. See Nardelli-Haefliger et al., “Immune Responses Induced by Lower Airway Mucosal Immunisation with a Human Papillomavirus Type 16 Virus-like Particle Vaccine,” Vaccine 23(28):3634-3641 (2005), which is hereby incorporated by reference in its entirety.

The pharmaceutical compositions of the present invention can also include an effective amount of an additional adjuvant. As noted above, papillomavirus VLPs and capsomeres are known to act as an adjuvant. Suitable additional adjuvants include, without limitation, Freund's complete or incomplete, mineral gels such as aluminum hydroxide, surface active substances such as lysolecithin, pluronic polyols, polyanions, peptides, oil emulsions, dinitrophenol, and potentially useful human adjuvants such as Bacille Calmette-Guerin, Carynebacterium parvum, and non-toxic Cholera toxin.

The present invention also relates to a method of inducing an immune response against RSV that includes administering a VLP or capsomere of the present invention or pharmaceutical composition of the present invention to an individual in an amount effective to induce an immune response against RSV.

It is contemplated that the individual to be treated in accordance with the present invention can be any mammal, but preferably a human. Veterinary uses are also contemplated. While the individual can be any mammal that is known to be infected by RSV, the RSV polypeptide incorporated into the VLPs or capsomeres is preferably derived from a genotype that is specific to a host mammal intended to be immunized in accordance with the present invention. For example, for treating humans it is preferable that the RSV polypeptide is derived from a human RSV strain. The individual to be treated is preferably an infant or juvenile, an elderly individual, or an individual having a cardiopulmonary or immunosuppressive condition.

Effective amounts of the composition will depend upon the mode of administration, frequency of administration, nature of the treatment, age and condition of the individual to be treated, and the type of pharmaceutical composition used to deliver the compound. Effective levels of the composition may range from about 0.001 to about 2.5 mg/kg depending upon the clinical endpoints and toxicity thresholds. While individual doses may vary, optimal ranges of the effective amounts may be determined by one of ordinary skill in the art.

The pharmaceutical composition can be administered by any means suitable for producing the desired immune response. Preferred delivery routes include orally, by inhalation, by intranasal instillation, topically, transdermally, parenterally, subcutaneously, intravenous injection, intra-arterial injection, intramuscular injection, intraplurally, intraperitoneally, or by application to mucous membrane. The composition can be delivered repeatedly over a course of time that achieves optimal enhancement of the immune response.

Exemplary modes of administration include a delivery vehicle that includes the composition of the present invention. Such delivery vehicles can be in the form of a single-unit oral dosage. Alternatively, the delivery vehicle can be in the form of a syringe comprising an injectable dose, in the form of a transdermal patch containing a transdermally deliverable dosage, or in the form of an inhaler containing an inhalable dosage.

For prophylactic treatment against RSV infection, it is intended that the composition(s) of the present invention can be administered prior to exposure of an individual to the RSV and that the resulting immune response can inhibit or reduce the severity of the RSV infection such that the RSV can be eliminated from the individual. For therapeutic treatment of active RSV infections, it is intended that the composition(s) of the present invention can be administered to an individual who is already exposed to the RSV. The resulting enhanced immune response is believed to reduce the duration or severity of the existing RSV infection, as well as minimize any harmful consequences of untreated RSV infections. The composition(s) can also be administered with any other therapeutic anti-RSV regimen.

### EXAMPLES

The following examples are provided to illustrate embodiments of the present invention but are by no means intended to limit its scope.

### Materials and Methods for Examples 1-7

### ELISA

Total IgG and IgG isotypes (IgG1, IgG2a) reactive with RSV F/G protein were determined (Murphy et al., “Dissociation Between Serum Neutralizing and Glycoprotein Antibody Responses of Infants and Children who Received Inactivated Respiratory Syncytial Virus Vaccine,” J Clin Microbiol 24:197-202 (1986); Wagner et al., “Serum Immunoglobulin G Antibody Subclass Responses to Respiratory Syncytial Virus F and G Glycoproteins After Primary Infection,” J Clin Microbiol 24:304-306 (1986), each of which is hereby incorporated by reference in its entirety). RSV F protein of sufficient quantity and purity were coated (100 ng/well) onto 96 well flat-bottom ELISA plates overnight at 4° C. (Walsh et al., “Purification and Characterization of the Respiratory Syncytial Virus Fusion Protein,” J Gen Virol 66 (Pt 3):409-415 (1985), which is hereby incorporated by reference in its entirety). The plates were then washed with PBS/0.5% Tween-20, incubated for 1 hr at RT with serial two fold dilutions of mouse sera in duplicate starting at 1:100 dilution, washed, and incubated with AP-conjugated goat α-mouse total IgG or AP-conjugated IgG subtype specific antisera (Southern Biotech) at 1:3000 dilution. Then the plates were again washed and the colorimetric reactions following addition of D-nitro-phenyl-phosphate (Sigma-Aldrich; 1 mg/ml in diethanolamine buffer) were read as OD₄₀₅ₙₘ using an automated ELISA plate reader (Dynex). Mean ODs for each dilution were plotted and the end point titer defined as the serum dilution yielding an OD>0.1 and ≧3 S.D. above the OD in wells without antigen.

### Immunoblots

Immunoblots were performed to determine whether the mouse sera contains anti-RSV F/G antibodies that will recognize the RSV F/G protein under various conditions (reducing or non-reducing, ±heating at RT, 56° C. or 95° C. 0.2% or 2% SDS). These conditions prior to SDS-PAGE will resolve the F protein as non-reduced, non-denatured trimer, as a non-reduced, non-denatured monomer (70 dD), or in a reduced, denatured state (50 and 20 kD fragments of F1 and F2, respectively) (Walsh et al. “Analysis of the Respiratory Syncytial Virus Fusion Protein Using Monoclonal and Polyclonal Antibodies,” J Gen Virol 67 (Pt 3):505-513 (1986), which is hereby incorporated by reference in its entirety). The proteins were transferred onto 0.2 μm NC membranes, incubated with each mouse serum sample (1:1000 in PBS-0.1% Tween 20/2% instant non-fat milk), and then incubated with HRP-conjugated goat α-mouse IgG (1:20,000). The interaction of various antisera to purified antigen will be visualized with the ECL (Pierce).

### Example 1

### Identification of a Full-length F Protein cDNA from Wildtype RSV Strain

A recent clinical RSV isolate (isolated in 1999; termed RGH strain, genotype A5) was expanded/plaque purified ×5 rounds using HEp-2 cells. RSV viral RNA was subsequently isolated from cell culture media and used as the template in RT-PCR; the initial RT step involved incubation of RNA at 42° C. for 1 hr with primer A (GCGGATCC, SEQ ID NO:117) and 10 U of AMV RT (Promega). An aliquot of the RT reaction was then mixed with primers A and B (GCGGATCC, SEQ ID NO:118), and Tli DNA polymerase (Promega) was used to PCR-amplify the F cDNA. The PCR amplicon was ligated into the BamHI site of pSP72 (Promega) to generate pR2-001. Both strands of the RGH strain F cDNA were sequenced (ABI PRISM 3730 DNA analyzer) and the entire F cDNA sequence was deposited into GenBank (Accession Number DQ885231, which is hereby incorporated by reference in its entirety).

### Example 2

### Comparative Analysis of the RGH Strain F Protein

The amino acid sequence of the RGH F protein was compared with that of two commonly used laboratory RSV strains, A2 and Long (both genotype A). The RGH F protein sequence has 97% (561/574 aa) identity with the F protein of RSV Long strain and 96% (556/574 aa) identity with that of the RSV A2 strain as measured by BLAST alignment. Importantly, amino acid sequences of the two CTL epitopes (residues 85-93 and 249-258, respectively) and all the amino acids involved in the binding of neutralizing antibodies (FIG. 1) in the RGH F protein are identical to those of the RSV Long strain. As previously described, the CTL epitope within amino acids 92-106 is found in the RSV A2 strain but not in the Long or RGH strains primarily due to the two changes at positions 105 and 106. Within the RGH F protein, the amino acid sequence between positions 226 and 447 differs from that of the Long strain at only three positions (3841-V, 400A-T, and 442V-A), none of which has been shown to be involved in binding of neutralizing antibodies. These observations indicate that the immune responses elicited by portions of the RGH F protein are not expected to be significantly different from those generated from the Long strain.

### Example 3

### Plasmid and Baculovirus Constructions

One approach for construction of fusion proteins was to identify portions of the F protein that: 1) were sufficiently small (≦150 aa) to be fused to the C termini of HPV capsid proteins without adversely perturbing VLP formation; 2) bore known epitopes for neutralizing antibodies and/or murine CTL responses; and 3) lacked the TM domain or long stretches of hydrophobic amino acids that may disrupt formation of VLPs. Three domains that met these criteria were initially identified: Domain 1, residues 23-122 of SEQ ID NO: 2; Domain 3, residues 226-378 of SEQ ID NO: 2; and Domain 4, residues 379-523 of SEQ ID NO: 2 (see FIG. 1). Also, Domain 2 (residues 154-222 of SEQ ID NO: 2) was chosen to be included in this experiment. Given the availability of appropriate plasmids and the likely possibility that a truncated L2 protein (L2N, residues 1-237 of HPV-16 L2) may accommodate longer foreign peptides than the full-length L2, initial efforts focused on generating chimeric L2N proteins, each fused at the C terminus to one of the RSV F domains described above.

To construct the baculovirus stocks designed to express the chimeric L2N derivatives, eight primers (four pairs) were designed, all of which bore an EcoR1 site at the 5′ end. Each primer pair was designed to match 18-23 nucleotide sequences within the RGH F cDNA of (SEQ ID NO: 1) that would generate PCR amplicons encoding one of the F domains. These primers were then used in PCR reactions (Platinum Taq; Invitrogen) using the full-length RGH F cDNA as the template. Each PCR amplicon was ligated into pCR2.1-TOPO (Invitrogen) and the sequence of each insert was verified. Each F cDNA fragment was then ligated into the EcoR1 site of pVL1392L2N. This plasmid, a derivative of the baculovirus transfer vector pVL1392 (Orbigen), has previously been constructed and has three features: 1) it directs the expression of HPV 16 L2N under the polyhedron promoter; 2) immediately 3′ to the last codon of L2N cDNA is an EcoR1 site which enables in-frame ligation of heterologous cDNA fragments; and 3) immediately 3′ of the EcoR1 site is an oligonucleotide sequence encoding the FLAG epitope (DYKDDDDK, SEQ ID NO:119) as a C terminus “tag” to immunologically recognize the L2N-RSV F chimeric proteins (Einhauer et al., “The FLAG Peptide, a Versatile Fusion Tag for the Purification of Recombinant Proteins,” J Biochem Biophys Methods 49:455-465 (2001), which is hereby incorporated by reference in its entirety). Following each plasmid construction, the orientation and sequence of the RSV F cDNA fragments were confirmed by sequencing.

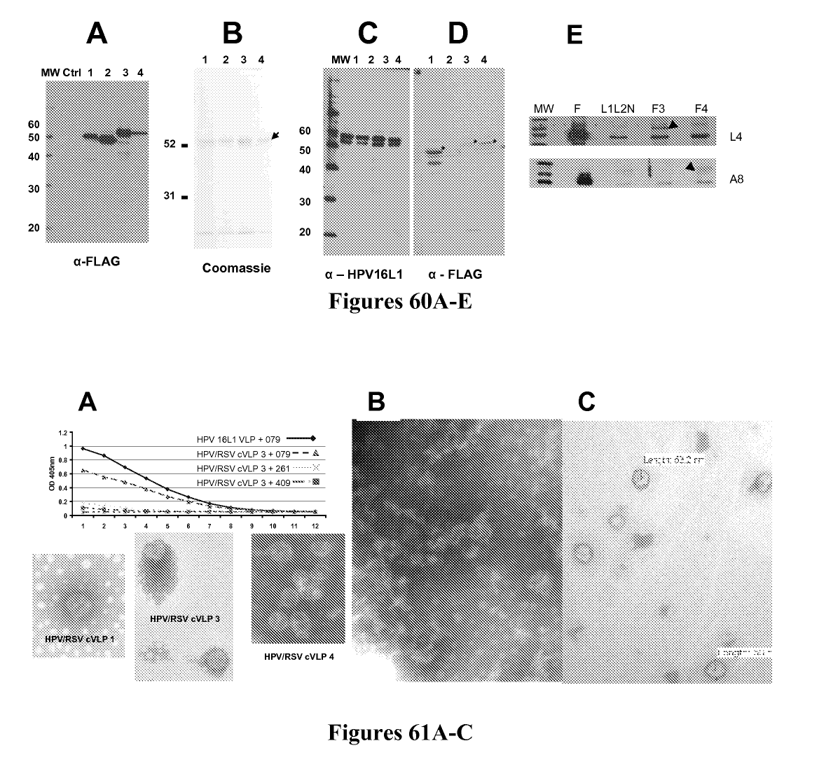

Derivatives of pVL1391L2N bearing portions of the RSV F cDNA were co-transfected into Sf9 cells with baculovirus DNA (Baculo-Gold; Pharmingen) and cellfectin (Invitrogen). After 72 hrs, the Sf9 serum-free media from each co-transfection was removed and the baculovirus stocks were serially propagated and plaque purified 3×. Sf9 cells infected at the final propagation step were assayed for the production of the L2N-RSV F fusion proteins (FIG. 60A-E). All four L2N-F chimeric proteins were expressed in Sf9 cells. As previously noted, the electrophoretic mobility of HPV L2 protein and its truncated derivatives is typically slower than the predicted size (i.e., L2N migrates with an apparent molecular weight of ˜40 kD) (Greenstone et al., “Chimeric Papillomavirus Virus-like Particles Elicit Antitumor Immunity Against the E7 Oncoprotein in an HPV16 Tumor Model,” Proc Natl Acad Sci USA 95:1800-1805 (1998); Kamper et al., “A Membrane-destabilizing Peptide in Capsid Protein L2 is Required for Egress of Papillomavirus Genomes from Endosomes,” J Virol 80:759-768 (2006); Finnen et al., “Interactions Between Papillomavirus L1 and L2 Capsid Proteins,” J Virol 77:4818-4826 (2003), each of which is hereby incorporated by reference in their entirety).

### Example 4

### HPV/RSV cVLP Formation and Characterization

Using the recombinant baculovirus of Example 3, HPV/RSV L1/L2N cVLPs were then generated. T. ni cells growing at log phase in 250 mL cultures (2×10⁶ cells/mL) were co-infected with an existing baculovirus stock that expresses full length HPV L1 protein and one of the four baculovirus stocks directing the synthesis of L2N-RSV F chimeric proteins. The multiplicity of infection (MOI) for all viruses was ≧3. After 72 hrs, the cells were collected by centrifugation, resuspended in ice-cold PBS+Complete Protease Inhibitor cocktail (Roche), and lysed using a Dounce homogenizer and a sonicator. The resulting mixture was brought to 40% CsCl in 1×PBS and subjected to four rounds of ultracentrifugation (3×40% CsCl, 1×40-60% sucrose gradient). Visible bands within the final CsCl isopycnic gradient were removed and dialyzed against Buffer N (PBS+0.5M NaCl) prior to −80° C. storage (Rose et al., “Human Papillomavirus (HPV) Type 11 Recombinant Virus-like Particles Induce the Formation of Neutralizing Antibodies and Detect HPV-specific Antibodies in Human Sera,” J Gen Virol 75(8):2075-2079 (1994); Rose et al., “Human Papillomavirus Type 11 Recombinant L1 Capsomeres Induce Virus-neutralizing Antibodies,” J Virol 72:6151-6154 (1998), each of which is hereby incorporated by reference in its entirety).

The resulting HPV/RSV L1/L2N cVLPs were tested for purity, structural integrity, and presence of conformation-dependent neutralizing epitopes on L1. Each cVLP preparation was primarily comprised of a protein doublet of 55-57 kD that was estimated to be >95% pure by Coomassie blue staining (FIG. 60B). Consistent with this observation, each cVLP preparation contained doublet bands of similar size that were detected on an immunoblot probed with a rabbit polyclonal antibody against 16L1 denatured epitopes (FIG. 60C) (Christensen et al., “Human Papillomavirus Types 6 and 11 Have Antigenically Distinct Strongly Immunogenic Conformationally Dependent Neutralizing Epitopes,” Virology 205:329-335 (1994); Christensen et al., “Immunization with Viruslike Particles Induces Long-term Protection of Rabbits Against Challenge with Cottontail Rabbit Papillomavirus,” J Virol 70:960-965 (1996), each of which is hereby incorporated by reference in its entirety). The two major bands seen on the protein gel and immunoblot most likely represent differentially glycosylated L1. When cVLP preparations (10× amount of protein/lane more than in FIG. 60B to compensate for the 30:1 L1:L2N ratio) were probed on an immunoblot with anti-FLAG mAb (Sigma-Aldrich), the L2N-RSVF-FLAG fusion proteins were detected in cVLPs 1, 3, and 4 (FIG. 60D). Similar results were obtained in immunoblots using the rabbit polyclonal HPV 16L2 antibody. Immunoblots to confirm the expression of RGH F protein moieties in the cVLPs show that for cVLP 3 and 4, the respective L2N-F proteins are detected by mAbs L4 and A8 (FIG. 60E). These mAbs recognize RSV F₁ subunit under denaturing conditions, are neutralizing and/or fusion-inhibiting, and L4 is protective against RSV infections (Walsh et al., “Analysis of the Respiratory Syncytial Virus Fusion Protein Using Monoclonal and Polyclonal Antibodies,” J Gen Virol 67(3):505-513 (1986), which is hereby incorporated by reference in its entirety.

ELISAs using α-RSV F Ab do not show significant reactivity in cVLPs, indicating that the cVLPs bear RSV F moieties that are likely to be localized internally to VLP capsid structures. In ELISAs, the presence of L1 neutralizing epitopes were detected in cVLP 3 (FIG. 61A) although there may be a slight reduction in the accessibility or conformationally-dependent detection of such epitopes secondary to the presence of the RSV-derived amino acid sequence (Rose et al., “Expression of the Full-length Products of the Human Papillomavirus Type 6b (HPV-6b) and HPV-11 L2 Open Reading Frames by Recombinant Baculovirus, and Antigenic Comparisons with HPV-11 Whole Virus Particles,” J Gen Virol 71(11):2725-2729 (1990); Rose et al., “Expression of Human Papillomavirus Type 11 L1 Protein in Insect Cells: In Vivo and In Vitro Assembly of Viruslike Particles,” J Virol 67:1936-1944 (1993); Giroglou et al., “Immunological Analyses of Human Papillomavirus Capsids,” Vaccine 19:1783-1793 (2001), each of which is hereby incorporated by reference in its entirety). Similar ELISA results were obtained with cVLPs 1 and 4.

When subjected to transmission electron microscopy (TEM) at 80,000×, cVLPs 1, 3, and 4 revealed a viral capsid-like structure that is very similar in morphology to that of intact HPV L1L2 VLPs (FIG. 61A (below ELISA results)) (Greenstone et al., “Chimeric Papillomavirus Virus-like Particles Elicit Antitumor Immunity Against the E7 Oncoprotein in an HPV16 Tumor Model,” Proc Natl Acad Sci USA 95:1800-1805 (1998); Rose et al., “Expression of Human Papillomavirus Type 11 L1 Protein in Insect Cells: In Vivo and In Vitro Assembly of Viruslike Particles,” J Virol 67:1936-1944 (1993), each of which is hereby incorporated by reference in its entirety.

Together, these data indicate that HPV/RSV cVLPs 1, 3 and 4 possess conformation-dependent L1 neutralization epitopes, contain L2N-RSF F fusion proteins of interest, and are of sufficient purity for use in mouse immunization experiments.

Conditions for baculovirus infection and cVLP preparations have been optimized such that 6-9 mg total cVLPs (at relatively high concentrations; 1-1.5 mg/ml) can be purified from 1 liter of T. ni cell cultures. However, during the course of such scale-up experiments, the cVLPs become significantly aggregated and the capsid morphology of cVLPs can become quite disrupted (FIG. 61B). This is likely due to the strong surface adsorptions and interactions among cVLP particles (Shi et al., “Stabilization of Human Papillomavirus Virus-like Particles by Non-ionic Surfactants,” J Pharm Sci 94:1538-1551 (2005), which is hereby incorporated by reference in its entirety). However, such structural disruptions appear not to be of significance in the presence of low (0.01%) concentrations of Tween-20 (FIG. 61C). This observation and optimization procedures using non-ionic detergents should improve the structural stability and antigenicity of cVLPs (Shi et al., “Stabilization of Human Papillomavirus Virus-like Particles by Non-ionic Surfactants,” J Pharm Sci 94:1538-1551 (2005), which is hereby incorporated by reference in its entirety).

These cVLP preparations will be reformulated with dilute nonionic detergents for use in full-scale immunogenicity experiments in mice. Mice will be immunized with 50 or 100 μg (50 μl total volume) of the cVLPs by intramuscular injection in the hind leg both with and without separate adjuvant. Mice will receive a priming dose at day 0 and a boost on day 14. On day 28, mice will be euthanized and exsanguinated, and serum samples obtained for immunoassays and neutralization studies. In addition, spleens will be harvested for analysis of CTL response.

### Example 5

### Chemical Cross-Linking of RSV-Derived Neutralizing Epitopes onto Surface-Exposed Basic Amino Acids of L1 Homomeric VLPs

A short peptide was designed bearing the following amino acid sequence: CGGNSELLSLINDMPITNDQKKLMSNNV (SEQ ID NO:120). This sequence is notable for an amino-terminal cysteine residue placed to facilitate cross-linking, followed by two glycine linkers that afford flexibility, and amino acid residues 254-278 of SEQ ID NO: 2 (RSV F protein). The RSV-derived sequence contains the binding site for L4, an RSV-neutralizing monoclonal antibody.

HPV-16 L1 VLPs with >100-fold molar excess of SMPH (succinimidyl-6-[β-maleimidopropionamido]hexanoate), an amine- and sulfhydryl-reactive hetero-bifunctional cross-linker, at room temperature. After removal of excess cross-linking agent, the surface-activated VLPs were then incubated with >200-fold molar excess of the peptide of SEQ ID NO: 120 for two hours at RT. Unreacted peptide was removed via dialysis in PBS.

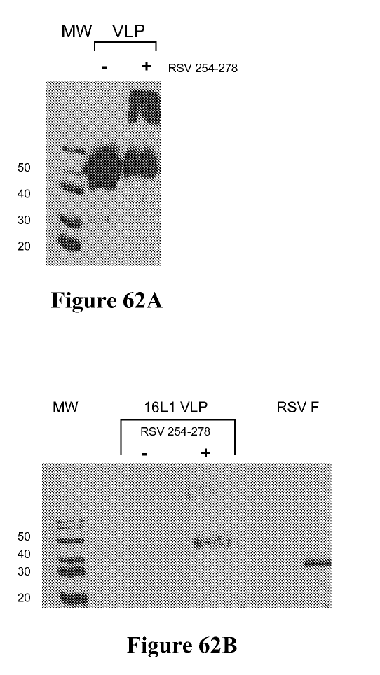

The resulting peptide-linked VLPs were then examined using anti-16L1 mAb (FIG. 62A) and anti-F mAb (L4; FIG. 62B). In FIG. 62A, the modified VLPs show higher-order aggregates near the top of the gel. Similar observations have previously been reported (Ionescu et al., “Pharmaceutical and Immunological Evaluation of Human Papillomavirus Like Particle as an Antigen Carrier,” J Pharm Sci 95:70-79 (2006), which is hereby incorporated by reference in its entirety), and presumably results from slower-migrating L1 derivatives bearing intra-molecular cross-links within L1 monomers.

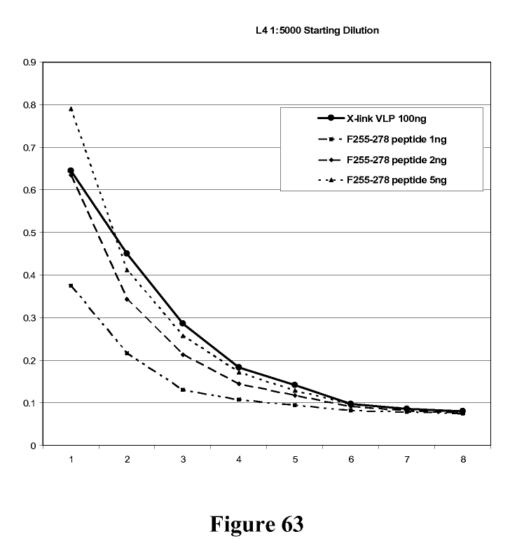

By immunoblots and ELISAs, the resulting cross-linked VLPs were recognized by the L4 mAb, confirming that there was successful cross-linking of the peptide onto L1 molecules, and that such cross-linking does not significantly alter the morphology of the VLPs (FIGS. 62B, 63). Based on the ELISA data, it appears that 100 ng of modified VLPs likely bear between 2-5 ng of the RSV peptide. By rough approximation, it appears that there are 135-335 peptide molecules cross-linked to each VLP.

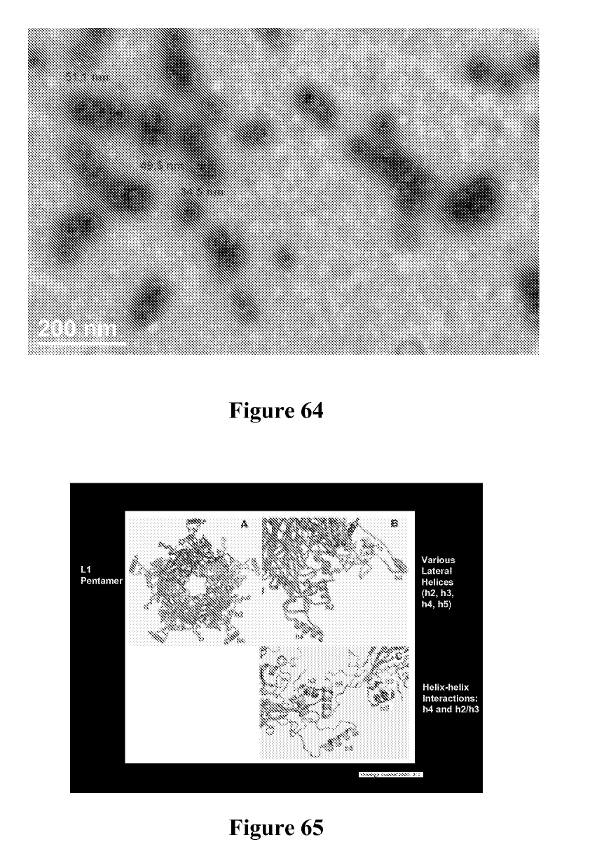

The cross-linked VLPs were also examined by transmission electron microscopy. As illustrated in FIG. 64, there appear to be two populations of VLPs based on particle size: the smaller (T=1 symmetry group) being approximately 30-35 nm in diameter and larger species (T=7 symmetry group) measuring approximately 50-60 nm across representative particles. Both VLP structures are thought to be immunogenic. Salt concentrations and pH are two factors that have previously been reported to alter the distribution of T=1 vs. T=7 structures within a given population of VLPs. It is likely, although not experimentally proven, that one or more steps of the cross-linking process may also affect the size distribution of the VLPs. As shown in electron microscopy (FIG. 64), the existence of individual VLPs as the predominant species following cross-linking argues against the possibility that these higher-order structures are due to inter-molecular or inter-VLP cross-linking.

After scaling the production of such cross-linked VLPs, purified, cross-linked VLPs will be used for mouse immunogenicity studies. Mice will be immunized with 50 or 100 μg (50 μl total volume) of the cross-linked VLPs by intramuscular injection in the hind leg both with and without separate adjuvant. Mice will receive a priming dose at day 0 and a boost on day 14. On day 28, mice will be euthanized and exsanguinated and serum samples obtained for immunoassay. A second group of mice will be challenged with RGH strain RSV to assess vaccine efficacy. In addition, spleens will be harvested for analysis of CTL response.

### Example 6

### Generation of Modified L1 Pentamers (Capsomeres) Bearing Antibody-Eliciting Epitopes and CTL Epitopes Within Surface-Exposed Loop Derivatives

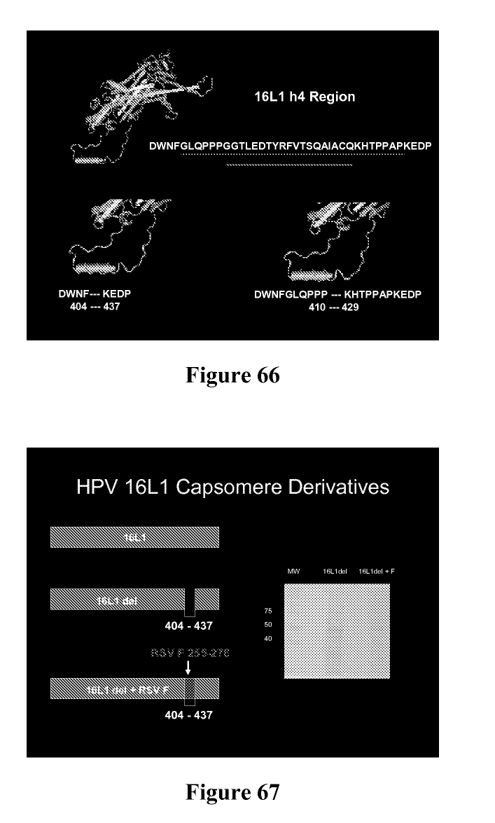

VLP-deficient chimeric L1 protein was prepared using L1 protein modified for deletion of the helix 4 domain, which abolishes VLP assembly (Bishop et al., “Structure-based Engineering of Papillomavirus Major Capsid L1: Controlling Particle Assembly,” Virol J 4:3, pp. 1-6 (2007), which is hereby incorporated by reference in its entirety) (see FIG. 65). Two versions of L1 capsid protein derivatives were generated, each bearing short deletions within the helix 4 (h4) domain, i.e., amino acid residues 404-437 and 410-429 of L1, respectively (FIGS. 66 and 67). These two versions are designated 16L1del1 and 16L1del2, respectively. Within one or both 16L1 helix 4 deletions, the following RSV-derived peptides were engineered RSV F residues 255-278 (SEQ ID NOS: 44 and 52), RSV F residues 423-436 (SEQ ID NOS: 46 and 54), RSV G residues 154-167 (SEQ ID NOS: 66 and 70), and RSV G residues 157-168 (SEQ ID NOS: 68 and 72). By way of example, SEQ ID NO: 44 is schematically illustrated in FIG. 67. The resulting L1 derivatives were synthesized in baculovirus-infected insect cells as described in Example 3, and purified over isopycnic CsCl centrifugation and sucrose cushions.

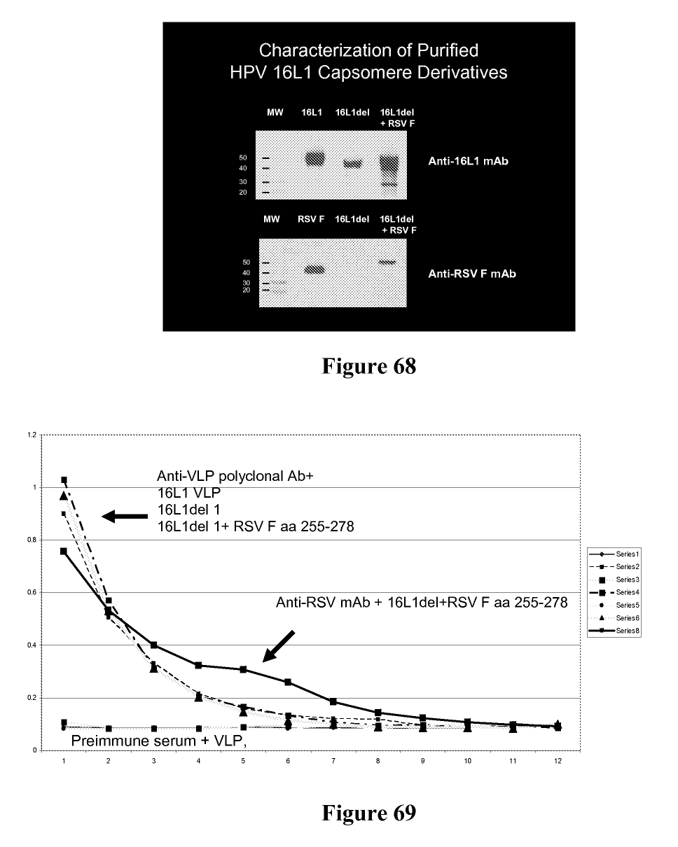

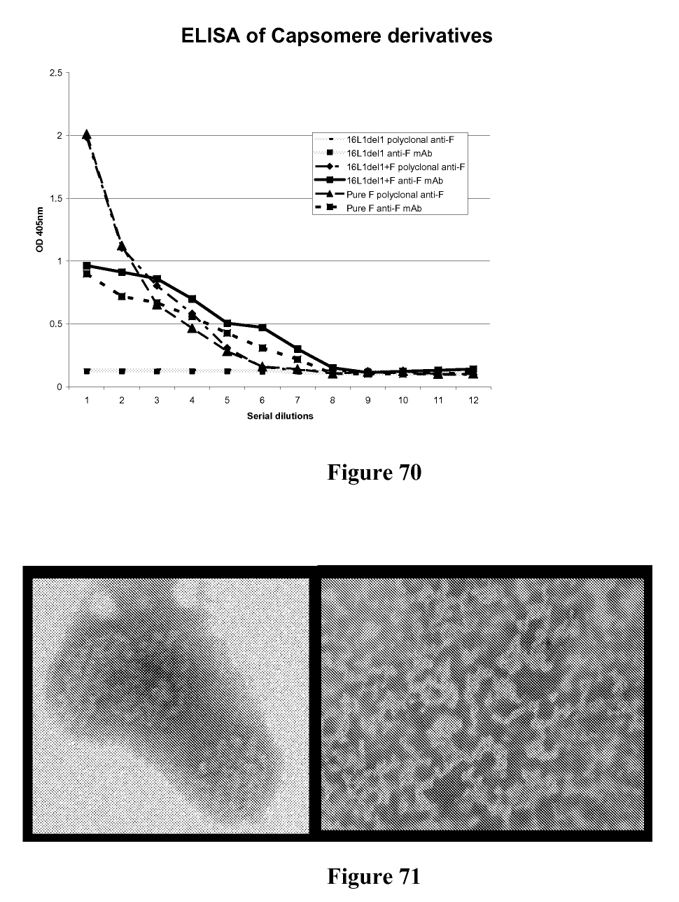

The biochemical, immunological, and structural aspects of the 16L1 h4 deletions and its derivatives were then examined. All of the 16L1 deletions/h4 epitope insertions appear to form characteristic circular capsomeric structures of approximately 7-10 nm in diameter (FIG. 71). Since the L4 mAb recognizes the RSV F amino acid residues 255-278, this antibody was used to characterize 16L1 h4 deletions bearing these RSV-derived polypeptide sequence. In immunoblots and ELISAs, both 16L1del1 (del aa 404-437) and 16L1del1+RSV F (aa 255-278) were recognized by anti-L1 antibodies, while only the latter protein was recognized by the L4 anti-F mAb (FIGS. 68-70).

The capsomeres produced were used for immunogenicity studies. BALB/c female mice (6-8 weeks old) were primed with intra-muscular (im) injections of Freund's complete adjuvant +100 μg of capsomere derivatives (1:16 L1del2; 2:16 L1del1+RSV F aa 423-436; 5:16 L1del2+RSV F aa 423-436; and 6:16 L1del1+RSV F aa 255-278) and boosted at weeks 3 and 6 with 50 μg im injections of each capsomere derivative in Freund's incomplete adjuvant. Week 10 terminal bleeds were analyzed by immunoblot and ELISA.

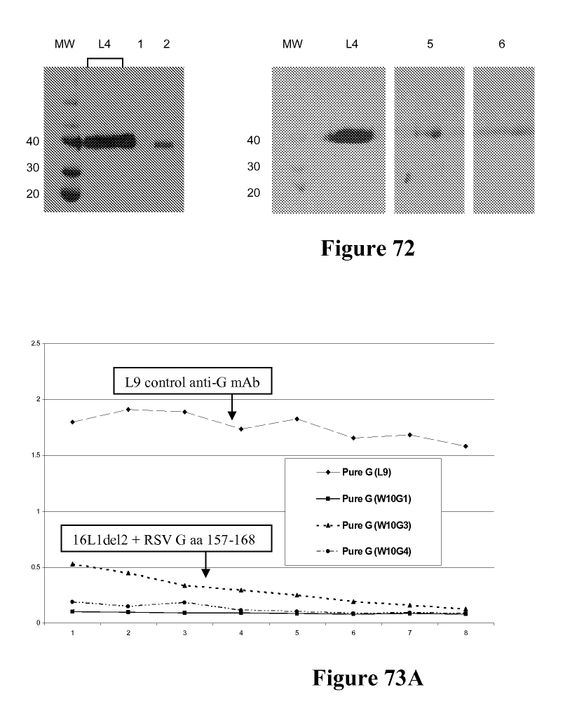

Immunoblots indicate that various sera from mice injected with capsomeres bearing RSV F-derived epitopes recognize purified RSV F protein under denaturing conditions (FIG. 72).

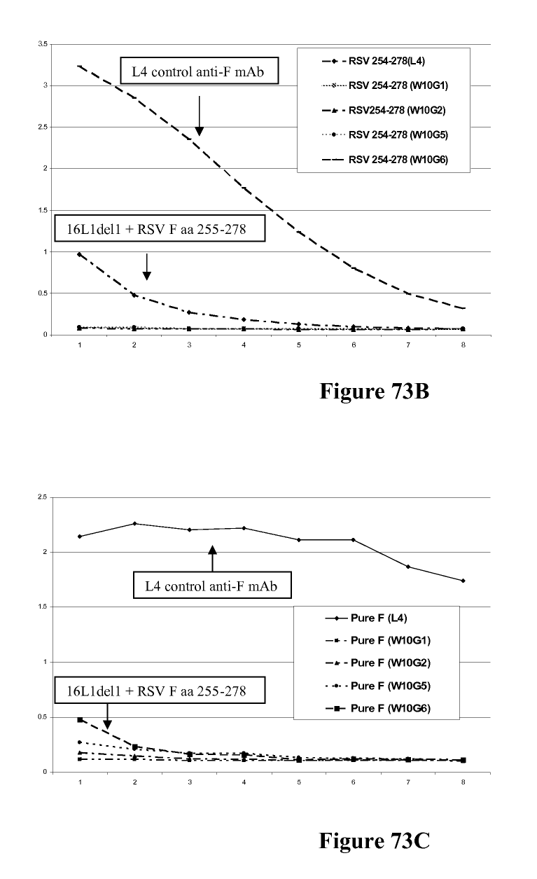

Furthermore, in ELISA assays, there is detectable recognition of purified RSV F and G proteins by sera from mice injected with appropriate capsomere derivatives. FIG. 73A-C shows representative ELISA assays to characterize Week 10 bleeds from several capsomere-injected mice. In FIG. 73A, 50-100 ng/well of purified RSV G protein was incubated with serially diluted anti-G mAb (L9; starting dilution 1:5,000) or pooled sera from mice injected with 16L1del2, 16L1del2+RSV G aa 154-167, or 16L1del2+RSV G aa 157-168 (starting dilutions at 1:200). The L9 mAb strongly recognizes RSV G protein while antisera from mice injected with 16L1del2+RSV G 157-168 shows limited but detectable interaction with purified RSV G protein. In FIG. 73B, 100 ng/well of peptide cross-linked to 16L1 VLPs (prepared in Example 5) was incubated with serially diluted anti-F mAb (L4; starting dilution 1:5,000) or pooled sera from mice injected with 16L1del2, 16L1del1+RSV F aa 423-436, 16L1del2+RSV F aa 423-436, or 16L1del1+RSV F aa 255-278 (starting dilutions at 1:200). The resulting OD405 nm were plotted as above. The L4 mAb strongly recognizes the RSV F-derived peptide and also the antisera from mice injected with 16L1del1+RSV F aa 255-278 shows significant interactions with the peptide. In FIG. 73C, 100 ng/well of purified RSV F protein was incubated with serially diluted anti-F mAb (L4; starting dilution 1:5,000) or pooled sera from mice injected with 16L1del2, 16L1del1+RSV F aa 423-436, 16L1del2+RSV F aa 423-436, or 16L1del1+RSV F aa 255-278 (starting dilutions at 1:200). L4 mAb strongly recognizes the RSV F-derived peptide while the antisera from mice injected with 16L1del1+RSV F aa 255-278 shows weaker but detectable interactions with the purified RSV F protein.

Taken together, these data indicate that capsomeres bearing RSV F or G-derived epitopes within the L1 h4 domain are immunogenic in mice.

Given their immunogenicity, a standard plaque reduction neutralization assay will be performed using these same pre- and post-immunization serum samples. Sera will be serially diluted starting at 1:25 in MEM/5% FCS. Each serum dilution (300 μL) will be mixed with 300 μL of MEM containing 215 plaque forming units (pfu) of RSV and incubated at RT for 30 min. An aliquot (200 μl) of each mixture will then inoculated onto preset HEp-2 monolayers in 24 well plates (Costar) for 2 hrs at RT. The inoculum will be removed and the monolayer overlayed with 2 ml of 0.5% methylcellulose in MEM/5% FCS and incubated for 4 days at 37° C. Plates will be fixed with 1 ml of 0.5% glutaraldehyde/PBS, washed, and stained with methylene blue. RSV plaques will be visualized and counted using a dissecting microscope. The neutralization titer is defined as the dilution (expressed as log₂ dilution) resulting in 50% plaque reduction compared to control wells containing virus without serum (Murphy et al., “Dissociation Between Serum Neutralizing and Glycoprotein Antibody Responses of Infants and Children Who Received Inactivated Respiratory Syncytial Virus Vaccine,” J Clin Microbiol 24:197-202 (1986); Falsey et al., “Serologic Evidence of Respiratory Syncytial Virus Infection in Nursing Home Patients,” J Infect Dis 162:568-569 (1990); Falsey et al., “Humoral Immunity to Respiratory Syncytial Virus Infection in the Elderly,” J Med Virol 36:39-43 (1992), each of which is hereby incorporated by reference in its entirety).

### Example 7

### Generation of HPV-16 L1N/RSV F Constructs

cDNA encoding a truncated L1 protein (L1N) will be generated using standard techniques. Basically, the full-length HPV serotype 16L1 cDNA will be used in PCR reactions to amplify a 1.4 kb cDNA encoding aa 1-495 of L1 and bearing EcoRI-XbaI (5′-3′ ends) restriction sites. The resulting amplicon will be ligated into the cognate sites within the MCS of pVL1391 to generate pVL1391-16L1N. The integrity of the 16L1N cDNA will be confirmed by sequencing. The above construction strategy provides a unique Xba1 site immediately following codon 495 of the 16L1N open reading frame.

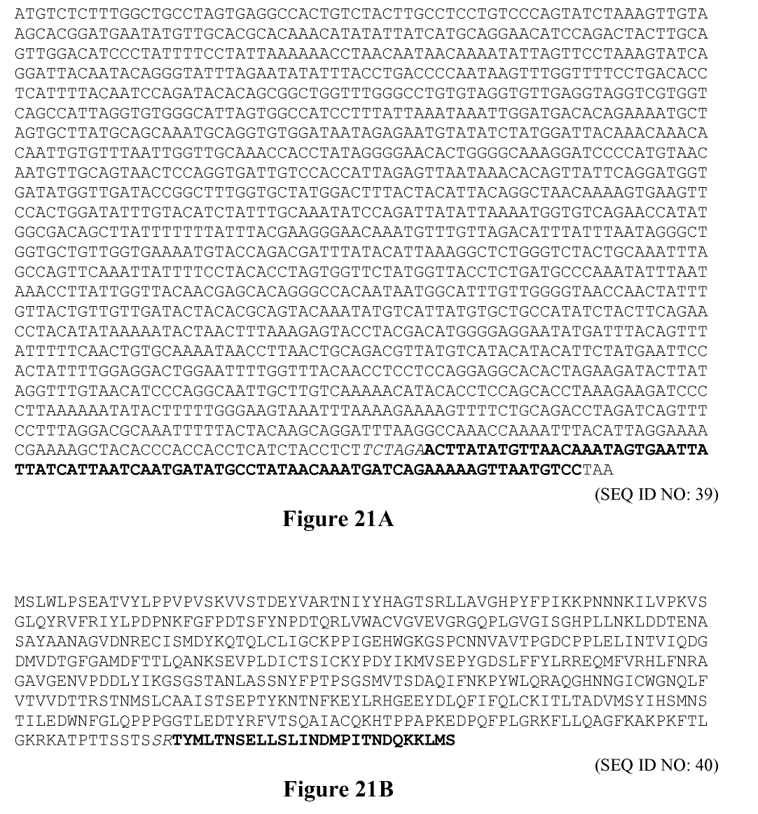

Then oligonucleotides will be used to generate the pVL1391-16L1N derivative bearing F sequences of interest. Two complementary oligos (each will be 86 nt in length, containing sense and antisense sequences encoding F protein aa 249-275 and bearing the 5 ′GGTCTAGA . . . (XbaI site italicized)), will be 5′ phosphorylated using polynucleotide kinase, annealed to form a stable duplex, and ligated into the XbaI site of pVL1391-L1N. One recombinant plasmid (pVL1391-16L1N-F) will be selected that has one copy of the oligo duplex ligated in the correct orientation. The amino acid sequence at the L1N-F junction is expected to be: STS (derived from L1)-RS (encoded by the XbaI site)-TYML (derived from RSV F) (see FIG. 21B, SEQ ID NO:40). The resulting plasmid will be used to generate the appropriate baculovirus stock to express the L1N-F chimeric protein. To detect the expression fusion protein in baculovirus-infected insect cell extracts, R409 (rabbit polyclonal antibody recognizing denatured L1 epitopes) and RSV neutralizing mAbs such as palimizumab will be used that recognize the portion of F fused to 16L1N.

Once the requisite baculovirus stocks are created, then T. ni cells in log phase growth will be infected with baculovirus directing the expression of L1N-RSV F amino acids 249-275, or co-infected with two baculovirus stocks, one directing the expression of L1N-RSV F amino acids 249-275 and the other expressing L2N bearing RSV F domains 1, 3, or 4. At 72 hrs following infection, cVLPs will be purified from the insect cells as described previously.

To ensure that the cVLPs are of sufficient quality and quantity to be used for subsequent experiments, each cVLP preparation will be subjected to: 1) SDS-PAGE followed by Coomassie blue staining; 2) measurement of cVLP protein concentration using a commercial colorimetric protein assay (BioRad); 3) ELISAs to ensure the presence of intact HPV L1 VLP epitopes in native conformation; and 4) presence of denatured epitopes on VLPs to be detected in immunoblots. Immunization studies will also be performed to assess immunogenicity and the sufficiency of the immune response to promote virus neutralization will be assessed via neutralization assay.

### Example 8

### Characterization of T-cell Response Following Immunization

Following immunization, it will also be examined whether or not CD4+ (Th1- or Th2-biased) and/or RSV-specific CTL responses accompany a neutralizing antibody response. To this end, the following assays will be performed: 1) intracellular cytokine staining (ICS) of splenocytes; 2) ELISA to determine the levels of IL-4, IL-5, and γ-IFN secreted by splenocytes; and 3) fluorescence-based CTL assay.

For each immunization group, the splenocytes will be harvested under sterile conditions using standard procedures and a 100 μm cell strainer will be used to generate single-cell splenocytes in PBS/1% FCS. The cells will then be resuspended in 5 mL hemolysis buffer (150 mM NH₄Cl, 1 mM KHCO₃, and 0.1 mM EDTA pH. 7.2-7.4) and thereafter washed ×3 with wash buffer before resuspension to 1×10⁶ cells/ml in RPMI 1640/10% FCS (Deml et al., “Virus-like Particles: A Novel Tool for the Induction and Monitoring of Both T-helper and Cytotoxic T-lymphocyte Activity,” Methods Mol Med 94:133-157 (2004), which is hereby incorporated by reference in its entirety).

For each immunization group, pooled splenocytes (2×10⁶ cells total) will be placed into 6 ml round-bottom tubes (Falcon) in duplicate. To ensure robust measurement of ICS signals, one set will be incubated for 2 hrs and the other will be incubated for 10 hours at 37° C. with UV-inactivated RGH strain RSV (10⁶-10⁷ pfu/ml) or media alone (Jackson et al., “Different Patterns of Cytokine Induction in Cultures of Respiratory Syncytial (RS) Virus-specific Human TH Cell Lines Following Stimulation with RS Virus and RS Virus Proteins,” J Med Virol 49:161-169 91996), which is hereby incorporated by reference in its entirety). Each sample will then be supplemented with 1 μl monensin (GolgiStop; BD) per tube for additional 6 hrs; based on this strategy, two time points will be obtained, one at 8 hrs and the other at 16 hrs, for each spleen sample/ICS. The cells will then be washed once in PBS/2% FCS and surface stained with either Quantum Red-conjugated rat α-mouse-CD4 or -CD8 mAb (Sigma) for 30 minutes at 4° C. Cells will then be washed, fixed and permeablized (Cytofix/Cytoperm; BD) and intracellularly stained using a commercially available kit (BD) with phycoerythrin-conjugated rat α-mouse IFN-γ antibody and rat α-mouse anti-IL-4-FITC antibody (BD). Cells will be analyzed using a 3-color FACscanner flow cytometer (BD FACScan) and CellQuest software (BD) (Deml et al., “Virus-like Particles: A Novel Tool for the Induction and Monitoring of Both T-helper and Cytotoxic T-lymphocyte Activity,” Methods Mol Med 94:133-157 (2004); Fischer et al., “Pertussis Toxin Sensitization Alters the Pathogenesis of Subsequent Respiratory Syncytial Virus Infection,” J Infect Dis 182:1029-1038 (2000); Rutigliano et al., “Treatment with anti-LFA-1 Delays the CD8+ Cytotoxic-T-Lymphocyte Response and Viral Clearance in Mice with Primary Respiratory Syncytial Virus Infection,” J Virol 78:3014-3023 (2004); Pala et al., “Flow Cytometric Measurement of Intracellular Cytokines,” J Immunol Methods 243:107-124 (2000), each of which is hereby incorporated by reference in its entirety).

For each immunization group, pooled spleens cells will be plated in triplicate into 96 well round bottom plates (2×10⁵ cells in 100 μl/well). The cells will be stimulated with UV-inactivated RGH strain RSV (10⁶ pfu/ml), purified RSV A2 F protein (100 ng/ml), phytohemagglutinin (PHA; Sigma-Aldrich) at 10 μg/ml, or media alone. Cells will be incubated at 37° C. with 5% CO₂ for 48 hours. Supernatants from each well will be harvested and assayed for secreted IFN-γ, IL-4, and IL-5 using an ELISA-based commercially available kit (BD) (Fischer et al., “Pertussis Toxin Sensitization Alters the Pathogenesis of Subsequent Respiratory Syncytial Virus Infection,” J Infect Dis 182:1029-1038 (2000); Rutigliano et al., “Treatment with anti-LFA-1 Delays the CD8+ Cytotoxic-T-lymphocyte Response and Viral Clearance in Mice with Primary Respiratory Syncytial Virus Infection,” J Virol 78:3014-3023 (2004), each of which is hereby incorporated by reference in its entirety).

For effector cells, splenocytes from each immunization group will be cultured in T25 flasks at 10⁶ cells/mL in RPMI/10% FCS. Each flask of cells will be stimulated ex vivo by addition of 10⁷ pfu of live RGH strain RSV and cultured at 37° C. and 5% CO₂ for 5 days. For target cells, BCH4 cells (derived from BAL/c embryo fibroblasts persistently infected with the Long strain of RSV) and B4 cells (a BALB/c fibroblast cell line uninfected with RSV) will be labeled (Fernie et al., “The Development of Balb/c Cells Persistently Infected with Respiratory Syncytial Virus: Presence of Ribonucleoprotein on the Cell Surface,” Proc Soc Exp Biol Med 167:83-86 (1981), which is hereby incorporated by reference in its entirety. BCH4 cells will be labeled with 5 μM 5-(and-6)-carboxyfluorescein diacetate, succinimidyl ester (CFSE) and B4 cells will be labeled with 0.5 μM CFSE. The labeled cells will then be washed with RPMI/10% FCS and plated onto 96 well plates (Nunc) at 20,000 cells/well in 100 μl media. Equal numbers (10,000 cells) of CFSE high and CFSE low target cells will be incubated simultaneously with the effector cells and incubated for 2-4 hrs at 37° C. The cells will then be analyzed by flow cytometry, and the percentage of RSV-specific target cell lysis will be calculated as 100−(% CFSE high cells/% CFSE low cells) (Rutigliano et al., “Identification of an H-2D(b)-restricted CD8+ Cytotoxic T Lymphocyte Epitope in the Matrix Protein of Respiratory Syncytial Virus,” Virology 337:335-343 (2005), which is hereby incorporated by reference in its entirety).

### Example 9

### Protective Efficacy of HPV/RSV Chimeric VLPs and Capsomeres Against RSV Challenge

As a final assessment of chimeric VLP or capsomere efficacy, mouse protection studies will be performed. First, the virus replication pattern for the RGH wild type RSV strain will be established. Mice (n=24) will be anesthetized with Ketamine (60-90 mg/kg) IP plus Xylazine (4-8 mg/kg) IP or acepromazine (1-2 mg/kg IP) and then be inoculated intranasally with 10⁶ pfu RGH RSV in 50 uL total volume of MEM/5% FCS from RSV-infected HEp-2 cells. On days 2, 3, 4, 5, 7, and 10, four mice will be weighed, sacrificed and subjected to bronchoalveolar lavage (BAL) and nasal wash (NW) using a 19-gauge blunt-end needle to inject ˜0.5 ml PBS/5% FCS into the trachea or nares (Walsh et al., “Protection from Respiratory Syncytial Virus Infection in Cotton Rats by Passive Transfer of Monoclonal Antibodies,” Infect Immun 43:756-758 (1984); Graham et al., “Primary Respiratory Syncytial Virus Infection in Mice,” J Med Virol 26:153-162 (1988), each of which is hereby incorporated by reference in its entirety). The samples will be centrifuged and virus titer determined by plaque assays using HEp-2 cells. The weights and plaque assay data will be plotted to determine the clinical manifestation of RSV infection and the kinetics of virus replication, respectively (Graham et al., “Primary Respiratory Syncytial Virus Infection in Mice,” J Med Virol 26:153-162 (1988), which is hereby incorporated by reference in its entirety).

Mice (6/group) will undergo two vaccinations (d0 and d14) with each of the chimeric VLPs or capsomeres that demonstrated an immune response that was effective for in vitro neutralization studies. Any modifications, such as use of adjuvant or altered amount of cVLPs/capsomeres in each injection, that were found to optimize immunogenicity of the cVLPs/capsomeres will also be used for this analysis. Negative and positive control mice will receive PBS or live RGH strain RSV, respectively. Four weeks (d42) later, mice will be challenged intranasally with 10⁶ pfu RGH RSV strain. When peak RSV viral titers are expected (as determined by viral kinetic experiments noted above), the mice will be sacrificed for BAL, and both NW and RSV titers will be measured as described above. As a qualitative measure of the severity of RSV infection, each animal will be weighed daily until sacrifice. Degree of protection by each of the cVLPs/capsomeres will be determined by comparison of the weights and viral titers to those of the negative control group. The initial choice of number of mice to be used is based on the expected minimum differences in viral titers in the immunized vs. non-immunized groups using the Student's t test. If deemed necessary based on the parametric/non-parametric distribution of data points, the Mann-Whitney rank sum test will also be used.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A chimeric papillomavirus virus-like particle (VLP) or capsomere comprising:
an L1 polypeptide and, optionally, an L2 polypeptide, and
a respiratory syncytial virus (RSV) protein or polypeptide fragment thereof comprising a first epitope, wherein the RSV protein or polypeptide fragment thereof is attached to one or both of the L1 and L2 polypeptides.

2. The chimeric papillomavirus VLP or capsomere according to claim 1, wherein the RSV protein or polypeptide fragment is attached via an in-frame gene fusion or a disulfide linkage to one or both of the L1 and L2 polypeptides.

3-4. (canceled)

5. The chimeric papillomavirus VLP or capsomere according to claim 1, wherein the L1 polypeptide comprises a deletion of at least a portion of a helix 4 domain.

6. (canceled)

7. The chimeric papillomavirus VLP or capsomere according to claim 1, wherein the L1 polypeptide is full length or a C-terminal or N-terminal L1 fragment, and the RSV protein or polypeptide fragment is attached via an in-frame gene fusion to an N-terminus, a C-terminus, or an internal position of the L1 polypeptide.

8. (canceled)

9. The chimeric papillomavirus VLP or capsomere according to claim 1, wherein the L2 polypeptide is an N-terminal L2 fragment and the RSV protein or polypeptide fragment is attached via an in-frame gene fusion to a C-terminal end of the L2 fragment.

10. The chimeric papillomavirus VLP or capsomere according to claim 1, which is in the form of a VLP.

11. The chimeric papillomavirus VLP or capsomere according to claim 1, which is in the form of a capsomere, and the L1 polypeptide is capsid-deficient.

12-18. (canceled)

19. The chimeric papillomavirus VLP or capsomere according to claim 1, wherein the RSV protein or polypeptide fragment comprising the first epitope is derived from an RSV protein selected from the group consisting of NS1, NS2, N, P, M, M2, L, SH, F, and G, and any combination thereof.

20-24. (canceled)

25. The chimeric papillomavirus VLP or capsomere according to claim 1, wherein the RSV protein or polypeptide fragment is attached via an in-frame gene fusion to the L1 polypeptide, and the L2 polypeptide further comprises an RSV protein or polypeptide fragment thereof comprising a second epitope.

26-27. (canceled)

28. The chimeric papillomavirus VLP or capsomere according to claim 25, wherein the RSV protein or polypeptide fragment comprising the second epitope is derived from an RSV protein selected from the group consisting of NS1, NS2, N, P, M, M2, L, SH, F, and G, and any combination thereof.

29-33. (canceled)

34. The chimeric papillomavirus VLP or capsomere according to claim 1, wherein VLP or capsomere comprises:
(i) an L1 polypeptide-RSV polypeptide chimeric protein comprising amino acid residues 23-122 of SEQ ID NO: 2, amino acid residues 154-222 of SEQ ID NO: 2, amino acid residues 226-378 of SEQ ID NO: 2, amino acid residues 379-523 of SEQ ID NO: 2, amino acid residues 379-559 of SEQ ID NO: 2, amino acid residues 249-275 of SEQ ID NO: 2, amino acid residues 254-278 of SEQ ID NO: 2, amino acid residues 255-278 of SEQ ID NO: 2, amino acid residues 423-436 of SEQ ID NO: 2, amino acid residues 154-167 of SEQ ID NO: 4, amino acid residues 157-168 of SEQ ID NO: 4, or a combination of any two or more thereof;
(ii) an L2 polypeptide-RSV polypeptide chimeric protein comprising amino acid residues 23-122 of SEQ ID NO: 2, amino acid residues 154-222 of SEQ ID NO: 2, amino acid residues 226-378 of SEQ ID NO: 2, amino acid residues 379-523 of SEQ ID NO: 2, amino acid residues 379-559 of SEQ ID NO: 2, amino acid residues 249-275 of SEQ ID NO: 2, amino acid residues 254-278 of SEQ ID NO: 2, amino acid residues 255-278 of SEQ ID NO: 2, amino acid residues 423-436 of SEQ ID NO: 2, amino acid residues 154-167 of SEQ ID NO: 4, amino acid residues 157-168 of SEQ ID NO: 4, or a combination of any two or more thereof; or
both (i) and (ii).

35. A pharmaceutical composition comprising a chimeric papillomavirus VLP or capsomere according to claim 1 and a pharmaceutically acceptable carrier.

36. (canceled)

37. The pharmaceutical composition according to claim 35 further comprising an effective amount of an adjuvant distinct of the VLP or capsomere.

38-39. (canceled)

40. A delivery vehicle comprising the pharmaceutical composition according to claim 35.

41-43. (canceled)

44. A method of inducing an immune response against respiratory syncytial virus (RSV) comprising:
administering a chimeric VLP or capsomere according to claim 1 to an individual in an amount effective to induce an immune response against RSV.

45. A method of preventing RSV infection comprising:
administering a chimeric VLP or capsomere according to claim 1 to an individual in an amount effective to prevent RSV infection.

46-52. (canceled)

53. The method according to claim 44, wherein said administering is also effective to induce an immune response against HPV.

54. A genetic construct encoding one or more chimeric proteins of claim 69.

55. The genetic construct according to claim 54, wherein the genetic construct comprises both the L1 polypeptide-RSV polypeptide chimeric protein and the L2 polypeptide-RSV polypeptide chimeric protein.

56-59. (canceled)

60. A recombinant vector comprising the genetic construct of claim 54.

61-65. (canceled)

66. A host cell comprising the recombinant vector of claim 60.

67-68. (canceled)

69. A chimeric protein comprising a papillomavirus L1 or L2 polypeptide and an RSV polypeptide linked via an in-frame gene fusion.

70. (canceled)

71. The chimeric protein according to claim 69, wherein the chimeric protein comprises the papillomavirus L1 polypeptide and the L1 polypeptide-RSV polypeptide chimeric protein comprises amino acid residues 23-122 of SEQ ID NO: 2, amino acid residues 154-222 of SEQ ID NO: 2, amino acid residues 226-378 of SEQ ID NO: 2, amino acid residues 379-523 of SEQ ID NO: 2, amino acid residues 379-559 of SEQ ID NO: 2, amino acid residues 249-275 of SEQ ID NO: 2, amino acid residues 254-278 of SEQ ID NO: 2, amino acid residues 255-278 of SEQ ID NO: 2, amino acid residues 423-436 of SEQ ID NO: 2, amino acid residues 154-167 of SEQ ID NO: 4, amino acid residues 157-168 of SEQ ID NO: 4, or a combination of any two or more thereof.

72. The chimeric protein according to claim 69, wherein the chimeric protein comprises the papillomavirus L1 polypeptide and the L1 polypeptide-RSV polypeptide chimeric protein comprises SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, or SEQ ID NO:72.

73. (canceled)

74. The chimeric protein according to claim 69, wherein the chimeric protein comprises the papillomavirus L2 polypeptide and the L2 polypeptide-RSV polypeptide chimeric protein comprises amino acid residues 23-122 of SEQ ID NO: 2, amino acid residues 154-222 of SEQ ID NO: 2, amino acid residues 226-378 of SEQ ID NO: 2, amino acid residues 379-523 of SEQ ID NO: 2, amino acid residues 379-559 of SEQ ID NO: 2, amino acid residues 249-275 of SEQ ID NO: 2, amino acid residues 254-278 of SEQ ID NO: 2, amino acid residues 255-278 of SEQ ID NO: 2, amino acid residues 423-436 of SEQ ID NO: 2, amino acid residues 154-167 of SEQ ID NO: 4, amino acid residues 157-168 of SEQ ID NO: 4, or a combination of any two or more thereof.

75. The chimeric protein according to claim 69, wherein the chimeric protein comprises the papillomavirus L2 polypeptide and the L2 polypeptide-RSV polypeptide chimeric protein comprises SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, or SEQ ID NO:116.

76. A method of making a chimeric VLP or capsomere comprising:
introducing a genetic construct into a host cell under conditions effective to express either (i) a fusion protein comprising an L1 polypeptide and RSV polypeptide; or (ii) an L1 polypeptide and a fusion protein comprising an L2 polypeptide and an RSV polypeptide, whereby the expressed polypeptide(s) self-assemble into the chimeric VLP or capsomere.

77. A method of making a chimeric VLP or capsomere comprising:
exposing a papillomavirus VLP or capsomere to a bi-functional linker molecule under conditions effective to allow covalent bond formation between the linker molecule and the VLP or capsomere, and
second exposing an RSV polypeptide to the product of said first exposing to allow covalent bond formation between the RSV polypeptide and the bound linker molecule, thereby forming the chimeric VLP or capsomere.

78-79. (canceled)
